(19) United States
(12) Patent Application Publication
(10) Pub. No.: US 2025/0195582 A1
(43) Pub. Date: Jun. 19, 2025

(54) IMMUNE MODULATORY PARACRINE ACTING CELLS (IMPACS)

(71) Applicant: Temple University - of the Commonwealth System of Higher Education, Philadelphia, PA (US)

(72) Inventors: Steven HOUSER, Philadelphia, PA (US); Hajime KUBO, Philadelphia, PA (US); Sadia MOHSIN, Philadelphia, PA (US); Alexander HOBBY, Philadelphia, PA (US)

(21) Appl. No.: 18/553,584

(22) PCT Filed: Apr. 1, 2022
(86) PCT No.: PCT/US2022/022989

§ 371 Date: Oct. 2, 2023

Related U.S. Application Data
(60) Provisional application No. 63/170,137, filed on Apr. 2, 2021.
(60) Provisional application No. 63/170,033, filed on Apr. 2, 2021.

(51) Int. Cl. A61K 35/34; A61K 31/7105; A61P 9/00; C12N 5/071; C12N 5/077; C12N 5/0775; C12N 15/113

(52) U.S. Cl. CPC A61K 35/34; A61K 31/7105; A61P 9/00; C12N 5/0656; C12N 5/0657; C12N 5/0662; C12N 5/069; C12N 15/113; C12N 2501/65

(57) ABSTRACT

Immune Modulatory Paracrine Acting Cells (IMPACS) and uses in the prevention or treatment of cardiovascular diseases and disorders thereof.

### CROSS REFERENCE TO RELATED APPLICATIONS

This Application claims the benefit of U.S. Provisional Application 63/170,137 filed on Apr. 2, 2021 and U.S. Provisional Application 63/170,033 filed on Apr. 2, 2021. The entire contents of these applications are incorporated herein by reference in their entirety.

### STATEMENT REGARDING FEDERALLY SPONSORED RESEARCH

This invention was made with government support under Grant Numbers 5R01HL139960-03 and 5P01HL134608-04 awarded by the National Institutes of Health. The government has certain rights in the invention.

### FIELD OF THE DISCLOSURE

Immune Modulatory Paracrine Acting Cells (IMPACs) and their uses in preventing, treating, regenerating cardiac tissues. Here, IMPACs and resulting Cardio Immune Modulatory Paracrine Acting Repair Therapy (CIMPACT, example included within) have immunomodulatory properties that promote wound healing.

### BACKGROUND

Coronary artery disease can lead to myocardial infarction (MI), a major cause of morbidity and mortality in the US. Cardiac ischemia and/or ischemia/reperfusion (IR) injuries cause death of the affected region of the heart, resulting formation of collagen based fibrous scar that reduces the contractile mass disrupting the pump function of the heart. If the loss of the contractile tissue exceeds the intrinsic compensative ability, the heart is driven into heart failure. Current standard of care for patients have been effective in restoring some cardiac function however cannot address the myocyte loss after injury. Stem cell therapies using numerous adult various Stem Cells have initially thought as a potential solution to regenerative therapies because of their inherent ability to differentiate into a variety of cell types. However, there is a consensus in the field that the beneficial outcome of cell therapy is not tied to neo myogenesis as initially thought instead can be attributed to modulating of wound healing response targeting inflammation, infarct expansion and subsequent scar formation.

### SUMMARY

Given the pervasiveness of cardiac disease, there exists a need for therapeutic cell replacement strategies utilizing transplantation of autologous and/or exogenous cells for the treatment or prevention of heart disease. Embodiments include cell therapy, immune evasion, conditioned paracrine mechanisms find the like for use in wound healing and cell therapies.

Provided herein are novel human Immune Modulatory Paracrine Acting Cells (IMPACs) that were developed by novel isolation and cultivation methods. Briefly, these cells have marker surface expression and secretory characteristics (as compared to mouse and pig Cortical Bone Stem Cells (CBSCs), mesenchymal stem cells (MSCs) and others) that enable them to display unique beneficial effects both in vitro and in vivo. Methods unique to IMPACs are further provided to isolate, purify and expand IMPACs. These methods cannot be used to isolate mouse or pig CBSCs or mesenchymal stem cells. IMPACs cannot be isolated, cultivated nor do they mature into MSCs using the standard/various isolation/cultivation protocols for MSCs, nor do mesenchymal stems cells mature into IMPACs. Accordingly, novel isolation and cultivation methods are provided herein for the isolation and cultivation of IMPACs are described. These novel cells when placed within an injured tissue are referred to as Cardio Immune Modulatory Paracrine Acting Cell Repair Therapy (CIMPACT) that enhance wound healing, such as after a myocardial infarction (MI), by modulating the formation of scar and protecting parenchymal cells (myocytes and blood vessels in the heart) from injury induced death.

Accordingly, in certain embodiments, a method of preventing or treating a cardiovascular disease or disorder comprises obtaining a biological sample comprising IMPACs; administering the IMPACs to a subject (CIMPACT), wherein the IMPACs differentiate into cardiac cells; thereby treating the cardiovascular disease or disorder.

In certain embodiments, a method of modulating an immune inflammatory response in wounded cardiac tissues and regeneration of these wounded tissues, comprises administering CIMPACT, a composition comprising IMPACs, IMPAC conditioned compositions or the combination thereof to a subject, wherein the compositions modulate the immune inflammatory response and promote wound healing and regeneration of these wounded tissues.

In embodiments, the biological sample comprises bone or fragments thereof. In certain embodiments, the IMPACs are isolated from the bone or fragments thereof.

In this and other embodiments, the isolated IMPACs are negative for one or more markers comprising: hematopoietic and lineage stem cell markers, CD34, CD 117, CD11b, CD31, CD45, major histocompatibility complex (MHC) class II molecules, MHC class Ia molecules, HLA-A, B, C or co-stimulatory molecules.

In this and other embodiments, the isolated IMPACs express CD49f (integrin alpha chain 6). In this and other embodiments, the isolated IMPACs express HLA-DQ.

In this and other embodiments, the IMPACs express embryonic stem cell surface markers during differentiation.

In this and other embodiments, the IMPACs express one or more markers during differentiation, wherein the one or more markers comprise SSEA-1, SSEA-4, TRA-1-60 or TRA-1-81.

In this and other embodiments, the IMPACs modulate immune inflammatory response and immune cell phenotypic shift.

In this and other embodiments, a method of treating or preventing cardiac disease or disorders, further comprises administering to the subject IMPAC exosomal micro RNAs (miRNAs).

In certain embodiments, the IMPACs and/or the IMPAC exosomal miRNAs are administrated systemically, locally or the combination thereof. In this and other embodiments, the IMPACs and/or the IMPAC exosomal miRNAs are administrated directly to damaged cardiovascular tissues.

In this and other embodiments, the IMPACs are obtained from sources comprising: autologous, allogeneic, haplotype matched, haplotype mismatched, haplo-identical, xenogeneic or combinations thereof.

In certain embodiments, a composition comprises isolated IMPACs, wherein the isolated IMPACs express CD49f (integrin alpha chain 6) and the composition comprises one or more factors to maintain the IMPACs in an undifferentiated state. In certain embodiments, the composition further comprises IMPAC exosomal micro RNAs (miRNAs). In this and other embodiments, the IMPACs are smaller in size when compared to human bone marrow stromal cells (hBMSC). In this and other embodiments, the IMPACs express soluble isoforms of HLA-G. In this and other embodiments, the IMPACs produce one or more immunoregulatory cytokines comprising: IL-4, IL-IRA, TGFβ, IL-10, IL-12α, IL-27R or combinations thereof.

In certain embodiments, a composition comprises isolated IMPACs and one or more IMPAC exosomal micro RNAs (miRNAs).

In certain embodiments, a IMPAC conditioned medium comprises one or more IMPAC exosomal micro RNAs (miRNAs), paracrine factors.

In this and other embodiments, the IMPAC exosomal miRNAs specifically inhibit one or more factors comprising: pro-inflammatory cytokines, anti-inflammatory cytokines, cytokines, transcription factors, toll-like receptor (TLR) pathway signaling molecules, NF-κB nuclear transporter, co-stimulatory molecules or combinations thereof.

In this and other embodiments, the pro-inflammatory cytokines comprise IL-6, TNF, IL-1α, IL-1β or combinations thereof.

In this and other embodiments, the anti-inflammatory cytokines comprise TGFβ2, TGFβ3 or the combination thereof.

In this and other embodiments, the cytokines comprise IL-2, IL-7, IL-13, IL-15 or the combination thereof.

In this and other embodiments, the transcription factors comprise: STAT1, STAT2, STAT3, STAT4, STAT5, STAT6 or combinations thereof.

In this and other embodiments, the TLR pathway signaling molecules comprise IRAK1, IRAK3, TRAF6, PDCD4, NF-kB1 or combinations thereof.

In this and other embodiments, the kNF-κB nuclear transporter is Importin-3a.

In this and other embodiments, the pro-inflammatory and co-stimulatory molecule is CD40LG.

In certain embodiments, a composition comprises isolated IMPACs and one or more cardiac cell types. In certain embodiments, the one or more cardiac cell types comprise: cardiomyocytes (CMs), fibroblasts (FBs), endothelial cells (ECs), peri-vascular cells or combinations thereof.

Other aspects are described in& .

### Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Unless specifically stated or obvious from context, as used herein, the term “about” is understood as within a range of normal tolerance in the art, for example within 2 standard deviations of the mean. About can be understood as within 10%, 9%, 8%, 7%, 6% 5% 4%, 3%, 2%, 1%, 0.5%, 0.1%, 0.05%, or 0.01% of the stated value. Unless otherwise clear from context, all numerical values provided herein are modified by the term about.

The term “cardiomyocyte” as used herein broadly refers to a muscle cell of the heart. The term cardiomyocyte includes smooth muscle cells of the heart, as well as cardiac muscle cells, which include also include striated muscle cells, as well as spontaneous beating muscle cells of the heart.

As used herein, the phrase “cardiovascular condition, disease or disorder” is intended to include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The terms “coronary artery disease” and “acute coronary syndrome” as used interchangeably herein, and refer to myocardial infarction refer to a cardiovascular condition, disease or disorder, include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The term “effective amount” as used herein refers to the amount of therapeutic agent of pharmaceutical composition, e.g., an amount of the synthetic modified RNA to express sufficient amount of the protein to reduce at least one or more symptom(s) of the disease or disorder, and relates to a sufficient amount of pharmacological composition to provide the desired effect. The phrase “therapeutically effective amount” as used herein, e.g., of IMPACs and/or IMPAC conditioned compositions as disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to, for example, effect a therapeutically or prophylactically significant reduction in a symptom or clinical marker associated with a cardiac dysfunction or disorder when administered to a typical subject who has a cardiovascular condition, disease or disorder.

With reference to the treatment of, for example, a cardiovascular condition or disease in a subject, the term “therapeutically effective amount” refers to the amount that is safe and sufficient to prevent or delay the development or a cardiovascular disease or disorder. The amount can thus cure or cause the cardiovascular disease or disorder to go into remission, slow the course of cardiovascular disease progression, slow or inhibit a symptom of a cardiovascular disease or disorder, slow or inhibit the establishment of secondary symptoms of a cardiovascular disease or disorder or inhibit the development of a secondary symptom of a cardiovascular disease or disorder. The effective amount for the treatment of the cardiovascular disease or disorder depends on the type of cardiovascular disease to be treated, the severity of the symptoms, the subject being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of a cardiovascular disease or disorder as discussed herein, for example treatment of a rodent with acute myocardial infarction or ischemia-reperfusion injury, and any treatment or administration of the compositions or formulations that leads to a decrease of at least one symptom of the cardiovascular disease or disorder as disclosed herein, for example, increased heart ejection fraction, decreased rate of heart failure, decreased infarct size, decreased associated morbidity (pulmonary edema, renal failure, arrhythmias) improved exercise tolerance or other quality of life measures, and decreased mortality indicates effective treatment. In embodiments where the compositions are used for the treatment of a cardiovascular disease or disorder, the efficacy of the composition can be judged using an experimental animal model of cardiovascular disease, e.g., as explained in detail in the examples section, or for example, animal models of ischemia-reperfusion injury (Headrick J P, Am J Physiol Heart Circ Physiol 285; H1797; 2003) and animal models acute myocardial infarction. (Yang Z, Am J Physiol Heart Circ. Physiol 282:H949: 2002; Guo Y, J Mol Cell Cardiol 33; 825-830, 2001). When using an experimental animal model, efficacy of treatment is evidenced when a reduction in a symptom of the cardiovascular disease or disorder, for example, a reduction in one or more symptom of dyspnea, chest pain, palpitations, dizziness, syncope, edema, cyanosis, pallor, fatigue and high blood pressure which occurs earlier in treated, versus untreated animals.

Subjects amenable to treatment by the methods as disclosed herein can be identified by any method to diagnose myocardial infarction (commonly referred to as a heart attack) or a cancer. Methods of diagnosing these conditions are well known by persons of ordinary skill in the art. By way of non-limiting example, myocardial infarction can be diagnosed by (i) blood tests to detect levels of creatine phosphokinase (CPK), aspartate aminotransferase (AST), lactate dehydrogenase (LDH) and other enzymes released during myocardial infarction; (ii) electrocardiogram (ECG or EKG) which is a graphic recordation of cardiac activity, either on paper or a computer monitor. An ECG can be beneficial in detecting disease and/or damage; (iii) echocardiogram (heart ultrasound) used to investigate congenital heart disease and assessing abnormalities of the heart wall, including functional abnormalities of the heart wall, valves and blood vessels; (iv) Doppler ultrasound can be used to measure blood flow across a heart valve; (v) nuclear medicine imaging (also referred to as radionuclide scanning in the art) allows visualization of the anatomy and function of an organ, and can be used to detect coronary artery disease, myocardial infarction, valve disease, heart transplant rejection, check the effectiveness of bypass surgery, or to select patients for angioplasty or coronary bypass graft.

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells). An “immune response” may typically be a specific reaction of the adaptive immune system to a particular antigen (so called specific or adaptive immune response) or an unspecific reaction of the innate immune system (so called unspecific or innate immune response), or a combination thereof.

“Micro-RNA” (“miRNA”) refers to small, noncoding RNAs of 18-25 nt in length that negatively regulate their complementary mRNAs at the posttranscriptional level in many eukaryotic organisms. See, e.g., Kurihara and Watanabe, Proc Natl Acad Sci USA 101(34):12753-12758 (2004). Micro-RNA's were first discovered in the roundworm C. elegans in the early 1990s and are now known in many species, including humans. As used herein, it refers to exogenously administered miRNA unless specifically noted or otherwise required by context.

As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

Unless specifically stated or obvious from context, as used herein, the term “or” is understood to be inclusive.

The terms “patient” or “individual” or “subject” are used interchangeably herein, and refers to a mammalian subject to be treated, with human patients being preferred. In some cases, the methods of the disclosure find use in experimental animals, in veterinary application, and in the development of animal models for disease, including, but not limited to, rodents including mice, rats, and hamsters, and primates. The terms include any animal that has, or is suspected of having, a myocardial injury, for example, myocardial ischemia. Suitable subjects (patients) include laboratory animals (such as mouse, rat, rabbit, guinea pig or pig), farm animals, sporting animals (e.g., dogs or horses) and domestic animals or pets (such as a horse, dog or cat). Non-human primates and human patients are included. For example, human subjects who present with chest pain or other symptoms of cardiac distress, including, e.g., shortness of breath, nausea, vomiting, sweating, weakness, fatigue, or palpitations, can be evaluated by a method of the invention. About ¼ of myocardial infarction (MI) are silent and without chest pain. Furthermore, patients who have been evaluated in an emergency room or in an ambulance or physician's office and then dismissed as not being ill according to current tests for infarction have an increased risk of having a heart attack in the next 24-48 hours; such patients can be monitored by a method of the invention to determine if and when they begin express markers of the invention, which indicates that, e.g., they are beginning to exhibit ischemia. Subjects can also be monitored by a method of the invention to improve the accuracy of current provocative tests for ischemia, such as exercise stress testing. An individual can be monitored by a method of the invention during exercise stress tests or Dobutamine stress tests to determine if the individual is at risk for ischemia; such monitoring can supplement or replace the test that is currently carried out. Athletes (e.g., humans, racing dogs or race horses) can be monitored during training to ascertain if they are exerting themselves too vigorously and are in danger of undergoing an MI.

Ranges provided herein are understood to be shorthand for all of the values within the range. For example, a range of I to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The terms “sample,” “patient sample,” “biological sample,” and the like, encompass a variety of sample types obtained from a patient, individual, or subject and can be used in a diagnostic, prognostic and/or monitoring assay. The patient sample may be obtained from a healthy subject, a diseased patient or a patient having associated symptoms of cardiovascular diseases or disorders.

Various methodologies of the instant invention include a step that involves comparing a value, level, feature, characteristic, property, etc. to a “suitable control,” referred to interchangeably herein as an “appropriate control,” a “control sample,” a “reference” or simply a “control.” A “suitable control,” “appropriate control,” “control sample,” “reference” or a “control” is any control or standard familiar to one of ordinary skill in the art useful for comparison purposes. A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype. For example, a “myocardial injury-positive reference level” of a biomarker means a level of a biomarker that is indicative of a positive diagnosis of myocardial injury in a subject, and a “myocardial injury—negative reference level” of a biomarker means a level of a biomarker that is indicative of a negative diagnosis of myocardial injury in a subject. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). Such reference levels may also be tailored to specific techniques that are used to measure levels of biomarkers in biological samples (e.g., LC-MS, GC-MS, ELISA, PCR, etc.), where the levels of biomarkers may differ based on the specific technique that is used.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

The term “tissue” refers to a group or layer of similarly specialized cells which together perform certain special functions. The term “tissue-specific” refers to a source or defining characteristic of cells from a specific tissue.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Any compositions or methods provided herein can be combined with one or more of any of the other compositions and methods provided herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic representation illustrating the isolation, production, growth of the cell and use in therapies.

FIGS. 2A-2F illustrate the IMPACs origin, isolation, cultivation and CIMPACT application for myocardium tissue repair. FIGS. 2A-2F are a series of photographs and graphs depicting the isolation, dimensions and phenotypes of the CBSCs.

FIG. 2A is a schematic representation and a series of photographs demonstrating the isolation of IMPACs.

FIG. 2B is a series of graphs showing the physical dimensions of IMPAC phenotypes. Cultured IMPAC phenotypes and hBMSCs (Stem Cell Technologies) were detached from the culture dish using Tryp LE Express (ThermoFisher). Immediately after the detachment, the live IMPAC were analyzed for their cell diameter and volume and compared with hMSCs. See, also Tables 1A and 1B.

FIG. 2C is a series of photographs depicting the IMPACs in suspension. Cultured IMPACs and hBMSCs were detached from the culture dish by Tryp LE Express (ThermoFisher). The diameter and volume of the cells were measured (n=20) immediately after the detachment. Scale bar: 20 μm for hBMSCs and 25 μm for IMPAC phenotypes.

FIG. 2D is a graph demonstrating that IMPACs are clonable and proliferate without senescence in culture. Single IMPACs sat various passage number were plated into a 96 well plate. The plated cells were then tracked for 7 days. The cell number in the well was counted every 24 h for the first 72 h and the doubling time determined.

FIG. 2E is a series of photographs showing cultures from day 0 to day 7 of IMPAC clones (p15 cells).

FIG. 2F is a series of photographs showing cultures from day 0 to day 7 of IMPAC clones (p80 cells).

FIGS. 3A and 3B are plots showing the surface markers of IMPACs. FIG. 3A depicts the surface marker characterization of IMPACs. FIG. 3B depicts the surface marker characterization of IMPACs differentiation to MSC like cells. Surface maker characterization of IMPACs. A total of 242 surface markers expressed on IMPAC phenotypes were screened. It was found that IMPAC phenotypes express very few surface markers. The surface markers detected on mCBSCs previously were not detected. Hematopoietic stem cell markers and lineage markers were also not detected. The IMPACs phenotypes were detected to express markers known to be expressed by pluripotent and multipotent stem cells (CD49f, SSAE-1, SSEA-4, TRA-1-61, and TRA-1-81). Significant levels of HLA-DQ, MHC class II molecule known to be expressed by antigen presenting cells were also detected. However, co-stimulatory factors (CD40, CD80, and CD86) that are necessary to activate immune response were not detected.

FIGS. 4A-4F are a series of schematics, graphs and scans demonstrating that CIMPACT preserves myocardial contractility and synchrony.

FIGS. 5A and 5B are a series of graphs and photographs demonstrating that CIMPACT injection attenuates structural remodeling and scar size.

FIG. 6A is an immunoblot demonstrating the immune modulation properties of IMPACs. Immunomodulatory proteins were detected in IMPACs. IMPACs were serum starved for 3 h and then stimulated with IL-1R (10 ng/ml), TNFα and INFγ (20 ng/ml each). The IMPACs were lysed at the baseline (0 h), 6 h or 24 h.

FIG. 6B is a series of immunofluorescence stains demonstrating the detection of immunomodulatory protein molecules.

FIG. 6C is a blot demonstrating that mouse, pig and IMPACs produce soluble forms of HLA-G. The major sHLA-G found in CBSCs and IMPACs was 25 kD HLA-G6.

FIG. 6D is an immunoblot demonstrating the immune modulation properties of IMPACs. Immunomodulatory proteins were detected in IMPACs. IMPACs were stimulated with IL-1β (10 ng/ml), TNFα and INFγ (20 ng/ml each), and then lysed at 0, 24 or 48 h. IMPACs responded to the inflammatory stimulation significantly increasing production of immunomodulatory proteins, IL-1RA, TGFβ, IL-4, and IL-10 (Data are presented as mean±SD; *p<0.05, **p<0.01, ***p<0.005, and ****p<0.001).

FIG. 6E is a graph demonstrating suppression of NK cytotoxic activity by IMPACs. Human natural killer (hNK) cell cytotoxicity assays were performed using LDH Cytotoxicity Assay Kit (LSBio) to see if the IMPACs have ability to evade hNK cell cytotoxicity. Target cells; IMPAC1.0 and K562 cells (Positive cytotoxicity control) were incubated for 4 h with hNK cells in two media; 10% FBS DMEM-F12 mix (Base Medium) and IMPAC1.0 conditioned medium (Condition Medium), supplemented with 20 ng/ml IL-2 to activate hNK cells, n=6. The target cell to hNK cell ratio we used was one target cell to five hNK cells. We found that hNK cell induced IMPAC death was significantly lower compared to K562 cell death (79.76±11.98% vs. 8.26±14.87%). Moreover, the K562 cell death was also significantly lower when incubated with IMPAC-CM (43.46±6.23%) indicating that IMPAC condition medium contains factors that suppress hNK cell cytotoxic activity (Data are presented as mean±SD; ***p<0.005, ****p<0.001).

FIG. 7A is a blot and a series of graphs demonstrating the effects of IMPACs co-culture on NRVM response to H₂O₂ oxidative stress. NRVMs and IMPACs were co-cultured using Boyden chambers. After 24 h of the co-culture, 0.6 mM H₂O₂ was added to the co-culture for 90 min and then analyzed for total Akt and Akt (Ser473) phosphorylation levels. It was found that the co-culture reduces the rate of Akt phosphorylation in NRVMs if the NRVMs were not under the oxidative stress (growth environment). On the other hand, the co-culture preserved total Akt abundance and increased the rate of Akt phosphorylation when the NRVMs were under the oxidative stress. These results indicate that the IMPACs secrete factors that bi-directionally regulate the cell survival pathways.

FIG. 7B is a series of graphs and photographs demonstrating that IMPAC secretomes suppress growth of endothelial cells. (1): IMPACs secrete factors that inhibit HUVECs growth. A single scratch to confluent HUVEC culture was made and the HUVECs were co-cultured with IMPAC phenotypes (2k, 4k, and 8k cells) and cultured with the 3-day condition media (CM). After 24 h incubation, dose dependent reductions in HUVECs growth were observed. (2): HUVECs were plated with IMPAC growth medium, CM, CM+DMSO, and CM+GW4869 (0.01 mM, an inhibitor of exosome generation). GW4869 significantly relieved the growth suppression effect of the CM indicating that the IMPAC exosomes play large role in the growth suppression effect of the IMPACs.

FIG. 7C is a series of photographs and a graph demonstrating that the IMPAC exosome promotes fibroblast growth. A single scratch to confluent mouse embryonic fibroblast (MEF) culture was made and then the MEFs were cultured with a growth medium (IMPAC medium), condition media (CM), and CM+GW4869 (0.01 mM). The CM significantly increased MEF growth. GW4869 significantly inhibited the CM induced increase in the MEF growth, indicating that the IMPAC exosomes play role in the growth effect of the IMPACs.

FIGS. 8A and 8B are a series of photographs demonstrating that the IMPAC secretome has immunomodulatory effects.

FIG. 8A: Human monocytes were isolated from peripheral blood mononuclear cells (hPBMNCs) and differentiated into macrophages by with Macrophage medium (Stemcell Technologies) supplemented with 50 ng/ml M-CSF.

FIG. 8B: The macrophages were treated with four treatments; 1) Control: Macrophage medium supplemented with 10% FBS; 2) CM (Condition medium); 3) Inflammation: Macrophage medium supplemented with 10% FBS and inflammatory cytokine cocktail (20 ng/ml TNFα, 20 ng/ml INFγ, and 10 ng/ml IL-1b); 4) CM+Inflammation: CM supplemented with the cytokine cocktail. The condition medium was obtained from 2 day culture of sCBSCs with Macrophage medium supplemented with 10% FBS. Additionally, 10 ng/ml M-CSF was added to each medium before use.

FIG. 9A is a blot demonstrating that IMPAC-CM reduces inflammatory stimulation induced increase in macrophage NFkB precursors.

FIG. 9B is a blot demonstrating that IMPAC-CM reduces macrophage NFkB nuclear translocation during inflammatory stimulation.

FIG. 9C is a series of blots demonstrating the effects of IMPAC-CM STATs abundance and activation.

FIG. 9D is a blot demonstrating that IMPAC secretomes elevate production of activated TGFβ in monocytes and NRVMs.

FIG. 9E demonstrates that IMPAC secretomes inhibit IL-6 production of macrophages, monocytes, and endothelial cells following an Inflammatory stimulation (Green: IL-6; Red GAPDH).

FIG. 9F demonstrates that IMPAC secretomes inhibit macrophage and endothelial IL-8 production during inflammatory stimulation.

FIG. 9G demonstrates that IMPAC-CM induced increase in CXCR4 abundance is inhibited during inflammatory stimulation in macrophages and monocytes.

FIGS. 10A-10C are a series of graphs, plots and photographs of Masson's Trichrome staining of excluded mice at 42-day post-I/R showing the scar sizes. The results demonstrated the CIMPACTs cell retention at Day3 and Day7. No hECs retention was observed at Day42 or earlier time points.

FIG. 11 is a series of plots demonstrating the effect of IMPAC exosomes on myofibroblast transition Adult rat ventricular fibroblasts were isolated from adult male Sprague Dawley rats. Fibroblasts were cultured overnight and then treated with IMPAC exosomes at increasing concentrations, +/−TGFBeta. Following 72 hrs of treatment, cells were fixed and stained for alpha-SMA. Intensity of alpha-SMA was quantified and shows a dose-dependent decrease of a-SMA in the fibroblasts, indicating that treatment with IMPAC exosomes decreases fibroblast transition to myofibroblasts, representing a potent antifibrotic response.

FIG. 12 is a table of exosome protein content characterization: Mass spectrometry analysis of exosome identified 155, 205, 238, and 48 proteins from IMPAC-1.0, IMPAC-2.0, IMPAC-3.0, and hBMSCs respectively. Of these proteins identified, 132 proteins were mutually inclusive within all of the hCBSAC phenotypes. Proteins that are identified at least 1% of total number of proteins identified to explore functional properties of IMPAC exosome. It was found that many of the identified proteins are inhibitory to fibrosis, coagulation, neovascularization, and inflammation. It was also found proteins that support fibroblast growth for the tissue regeneration and cell survival processes.

FIG. 13 is a table of the tumor suppressor micro RNAs found within IMPAC exosomes. IMPAC exosomes had enriched amount of tumor suppressive micro RNAs. Listed micro RNA are five most abundant micro RNA families (miR-122, miR-92, let-7, miR-103/107, and miR-199) found within IMPAC exosomes. Other abundant tumor suppressive micro RNA found were miR-320, -148, -99, -25, -31, -369, -26, -100, -23, -192, -143, -151, -30, -22, -140, -361, -125, -378, -541, -409, -183, -129, -130, -101, -410, -28, and -7 (order by abundance).

FIG. 14 is a table of anti-angiogenic micro RNAs found within IMPAC exosomes.

FIG. 15 is a table of cardio-protective (miR-21) and anti-fibrotic (miR-486) micro RNAs found within IMPAC exosomes.

FIG. 16 is a table of microRNA involved in anti-inflammatory and macrophage M2 polarization found within IMPAC exosomes.

FIG. 17 is a table of microRNA involved resolution of inflammation found within IMPAC exosomes.

FIG. 18 is a table of microRNA involved in down regulation of inflammatory signals found within IMPAC exosomes.

FIGS. 19A-19E are a series of graphs demonstrating the effect of IMPAC xenotransplantation into the border zone of myocardial infarction reperfusion injury: Cardiac function measured by echocardiography (Upper row). Animals underwent sham, MIR+PBS or MIR+CIMPACT injection (immediately after MIR or 24 h after the MIR) surgeries and received follow-up serial echocardiography at 1, 3, 7, and 14 days post-MIR. Functional parameters derived from echocardiography are shown; end-diastolic volume (FIG. 19A); and ejection fraction (FIGS. 19B and 19C). The MIR injured hearts that received immediate treatment of CIMPACT had improved cardiac function and structure relative to PBS treated control. Animals that received the CIMPACT 24 h after the MIR did not have the same level of beneficial effect. Infarct size analysis of CIMPACT phenotypes were performed at 3-day post MIR (FIG. 19D) and 14-day post MIR (FIG. 19E). Their area at risk or infarct area was determined using Evan's Blue or triphenyltetrazolium chloride (TTC) staining, respectively, and results are reported as a percentage of total ventricular area. CIMPACT reduced infarct area in all groups.

FIG. 20 is a series of tables and a graph showing that the mCBSC and IMPAC have different secretome profiles.

FIG. 21 is a series of blots and graphs showing the effects of mCBSC and IMPACs secretome on NRVM Response to H₂O₂ oxidative stress. NRVMs were treated with mCBSC or IMPAC secretome prior to H₂O₂ treatment (90 min). The NRVMs were then analyzed for total Akt and Akt (Ser473) phosphorylation levels. It was found that the IMPAC secretome reduced the rate of Akt phosphorylation in NRVMs if the NRVMs were not under the oxidative stress (growth environment). The IMPACs secretome preserved total Akt abundance and increased the rate of Akt phosphorylation when the NRVMs were under the oxidative stress. These results indicate that the IMPACs secrete factors that bi-directionally regulate the cell survival pathways. In contrast, mCBSC secretome reduced Akt phosphorylation in NRVMs under the oxidative stress, indicating that the mCBSC does not secrete the cardioprotective factors.

FIG. 22 is a graph showing that the mCBSC and IMPACs have different secretome profiles.

FIGS. 23A and 23B are immunostains demonstrating that injected hECs do not survive within mouse MI border zone while IMPACs survive at least for 42 days, indicating that IMPACs have ability to evade mouse immune cells.

FIGS. 24A-24C are a series of immunostains, graphs, plots and a schematic demonstrating that IMPAC treatment modulates post Myocardial I/R immune responses, reducing post MI immune cell infiltration (FIG. 24A) and promote macrophage M2 phenotypic shift (FIG. 24B). IMPAC secretome induced promotion of M2 macrophage phenotype and phargocytotic activity were confirmed in vitro using mouse bone marrow derived macrophages (FIG. 24C). Data are presented as mean±SD. *p<0.05, **p<0.01, ***p<0.005, ****p<0.001.

FIG. 25 is a series of immunostains and graphs demonstrating that injected IMPACs at mouse post myocardial I/R border zone promotes reduction of neutrophil infiltration and increased rate of galactin-3+M2 macrophage phenotype. Data are presented as mean±SD; *p<0.05 and ***p<0.005.

FIG. 26 is a series of immunostains and graphs demonstrating that IMPAC treatment induces early initiation and withdrawal of myofibroblast differentiation within the infarct zone indicating that IMPAC treatment promotes early initiation and completion of the post MI myocardial repair.

FIGS. 27A-27C are a series of immunostains and blots demonstrating one of IMPAC mechanism of actions. Injected IMPACs secrete Thrombospondin-1 (TSP-1) (FIG. 27A). IMPACs also secrete TSP-1 but not TSP-2 and activate TGFb in culture. IMPAC-CM contained abundant TSP-1 and active form of TGFb (dimer) (FIG. 27B). IMPAC conditioned medium treated hcFibroblasts maintained TSP-1/TGFβ signaling pathway molecules TSP-1, pSmad-2 and collagen-I abundance during inflammatory stimulation (FIG. 27C).

FIG. 28 is a blot demonstrating that IMPAC-CM treatment suppresses the inflammation induced hcFibroblast MMP3 production. Inhibition of TSP-1, one of major IMPAC secretomes, negates this inhibition indicating that TSP-1 secreted from IMPAC could inhibits hcFibroblast MMP3 production induced by inflammatory stimulation.

### DETAILED DESCRIPTION

Ischemic heart disease (IHD) is a leading cause of morbidity and mortality in Western Society. IHD causes death of myocytes and supportive tissue in affected regions of the heart. There is an unmet clinical need to develop therapies that can reduce the injury caused by myocardial infarction (MI) and to enhance repair. The resulting MI induced injury can lead patients to enter the Heart Failure journey, which is the largest economic burden to the U.S. health system (>$30B per year), with ˜50% of patients experiencing mortality within 5 years. Within the last 10 years, cell therapies to restore cardiac structure and function after MI has produced little to no improvement in cardiac function. Possible reasons for the lack of beneficial effects likely include the fact that the cells used to date do not have essential features to repair the injured heart.

In the examples section which follows, results from mice and pigs demonstrate that CBSCs delivered to the heart after ischemia/reperfusion MI can reduce scar size, enhance cardiac myocyte survival and improve cardiac function. Further provided are human cells derived from human bone stroma. The IMPACs have been isolated and cultivated by unique isolation, cultivation methods that differ from CBSCs from other species. The IMPACs were shown herein, to have surface expression and secretory characteristics (versus mouse and pig CBSCs, mesenchymal stem cells (MSCs) and others) that enable them to display unique beneficial effects both in vitro and in vivo. The isolation and cultivation methods developed for IMPACs were found to be unique to human derived cells and cannot be used to isolate mouse or pig CBSCs as these cells did not survive nor thrive using the methods for IMPACs. These methods were also unsuccessful when attempting to isolate mesenchymal stem cells as these cells did not survive nor thrive nor mature into IMPACs. In fact, MSCs died relatively early in the passage phase. In contrast to MSCs the IMPACs cannot be isolated, cultivated nor do they mature into MSCs using the standard/various isolation/cultivation protocols for MSCs. Accordingly, a novel human cell is provided for which was isolated and cultivated based on novel isolation and cultivation methods.

In addition, post isolation/cultivation of these cells demonstrated that these novel cells display unique surface characteristics (e.g., protein expression profile) and secrete a unique set of anti-inflammatory, immune modulatory, cell protective and angiogenic factors that again clearly differentiate them from both mouse and pig CBSCs and MSCs and others. Moreover, it was also demonstrated that IMPACs can survive in harsh conditions that mimic damaged tissues, secrete cardioprotective and wound healing factors, possess characteristics that reduce infarct size and improve cardiac function in mice with myocardial infarction. IMPACs also have surface characteristics that should make them immune privileged, which is likely why they survive in the infarcted heart for longer than other cell types.

These properties are described in detail in the examples section which follows, and demonstrate that the IMAPCs grew indefinitely and exhibited a potential to evade hNK and killer T cells. IMPACs secreted factors that suppressed overly activated inflammatory responses in immune cells and myocardial cells. The secreted factors exhibited a cardio protective effect against H₂O₂ induced oxidative stress bi-directionally. When injected into a mouse myocardial I/R border zone survived at least for 6 weeks, reduced neutrophil infiltration, scar formation and improved myocardial function. CIMPACT injected into a mouse myocardial I/R border zone exhibited a limited ability to trans-differentiate into myocardial cells forming IMPAC derived vascular structures and a small number of cardiac myocytes. Although IMPACs were detected to produce key immune moderately factors, IMPACs did not produce angiogenic factors. Contrary, IMPACs secreted more abundant miRNAs contained in exosomes compared to hMSCs that have potential to support the post MI reparative processes indicating that the exosomal miRNA may be the major paracrine factor for the CIMPACT induced reparative effect. These findings provided evidence that IMPACs have ability to survive within host myocardium for a long period of time and rectify deranged cellular and myocardial environment to re-establish the normal homeostasis. Thus, IMPACs would be a strong candidate for an allogeneic cell therapy targeting not only for myocardial infarction but also many other inflammatory diseases with unlimited cell supply potential.

Accordingly, embodiments, include cell isolation methods, cell cultivation methods, cell therapeutics, immune evasion, paracrine mechanisms for use in wound healing and cell therapies.

### Methods of Treatment

In certain embodiments, the methods described herein involve intramyocardial transplantation of IMPACs. Such therapeutic methods may repair and regenerate damaged myocardium and restore cardiac function after, for example, acute myocardial infarction and/or other ischemic or reperfusion related injuries. Methods generally include contacting cardiac tissues with a composition comprising IMPACs, IMPAC conditioned compositions or the combination thereof. Contacting may occur via injection methods known in the art and described herein.

In certain embodiments, a method for restoring cardiac function comprises introducing an effective amount of a composition comprising IMPACs, IMPAC conditioned compositions or the combination thereof, and a pharmaceutically acceptable carrier into a heart of a subject in need thereof. Restoration of cardiac function may include partial or complete restoration. In one embodiment, at least 50% of cardiac function is restored compared to a patient who does not receive such treatment. In another embodiment, about 50%, 60%, 70%, 75%, 80%, 85%, 90%, 95%, or 100% of cardiac function is restored. A subject receiving treatment may also be tested in various ways for cardiac health and have an improved result observed by echocardiography, multi-gated acquisition scan (MUGA) scan, nuclear stress test, radionuclide angiography, left ventricular angiography, MRI or ECG. In one embodiment, a patient's cardiac function does not worsen.

In certain embodiments, a method of inducing cardiomyocyte regeneration, cardiac repair, vasculogenesis or cardiomyocyte differentiation, comprises contacting a composition comprising IMPACs, IMPAC conditioned compositions or the combination thereof with injured heart tissue. In one embodiment, the IMPACs represent at least about 75%, at least about 80%, at least about 85%, at least about 90%, at least about 95%, at least about 98%, at least about 99%, or 100% of the cells in the composition.

In such methods, a subject may be diagnosed with, or at risk for, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, or myocardial hypertrophy. In one embodiment, the subject is diagnosed with myocardial infarction. In another embodiment, the subject has, or is at risk for, heart failure.

Where compositions such as those described herein are utilized for treatment of a subject, introducing or contacting the composition with the heart of the subject can occur by implanting the composition into cardiac tissue of the subject. Alternatively, introducing or contacting the composition can occur via injecting the composition into the subject using conventional techniques in the art. Cardiac tissue to be treated according to the present methods includes, for example, myocardium, endocardium, epicardium, connective tissue in the heart, and nervous tissue in the heart. Animals such as mammals represent subjects to be treated with the presently disclosed compositions and methods. In one embodiment, the subject is a human, a veterinary animal, a primate, a domesticated animal, a reptile, or an avian. For example, a human subject may be treated with the disclosed compositions to restore cardiac function and to treat one or more heart-related conditions.

In certain embodiments, a method of preventing or treating a patient suffering from myocardial infarction, chronic coronary ischemia, arteriosclerosis, congestive heart failure, dilated cardiomyopathy, restenosis, coronary artery disease, heart failure, arrhythmia, angina, atherosclerosis, hypertension, or myocardial hypertrophy comprises administering a composition comprising IMPACs, IMPAC conditioned compositions or the combination thereof, and a pharmaceutically acceptable carrier.

Generally, a subject upon which the methods of the invention are to be performed will have been diagnosed with myocardial infarction, chronic coronary ischemia, arteriosclerosis, congestive heart failure, dilated cardiomyopathy, restenosis, coronary artery disease, heart failure, arrhythmia, angina, atherosclerosis, hypertension, or myocardial hypertrophy. Alternatively, it will have been determined that a subject upon which the methods of the invention are performed is at risk for myocardial infarction, chronic coronary ischemia, arteriosclerosis, congestive heart failure, dilated cardiomyopathy, restenosis, coronary artery disease, heart failure, arrhythmia, angina, atherosclerosis, hypertension, or myocardial hypertrophy based on assessment of the heart tissue and/or family history. In one embodiment, a subject has been diagnosed with myocardial infarction or at risk for heart failure.

One in this field of endeavor would understand that methods of prevention are intended for subjects that have a family history of heart attacks or may physically be predisposed to heart attacks. Thus, prevention encompasses administration of compositions embodied herein to a subject to prevent damage to the subject's heart and/or to prevent acute myocardial infarction. A subject that has been treated with such methods may experience an overall improvement in health. Additionally, cardiac function may be restored and/or improved as described above compared to lack of treatment.

In accordance with one embodiment, a composition comprising IMPACs, IMPAC conditioned compositions or the combination thereof, is introduced into the cardiac tissue or cells a subject. Briefly, this method may be performed as follows. IMPACs are isolated as described in US 2016/0152952 incorporated herein by reference in its entirety. Once isolated, the IMPACs can be purified and/or expanded. The isolated cells can then be formulated as a composition (medicament) comprising the IMPACs, IMPAC conditioned compositions or the combination thereof, along with, for example, a pharmaceutically acceptable carrier. The composition (medicament) so formed can then be introduced into the heart tissue of a subject.

A subject to be treated with the disclosed compositions and methods will have been diagnosed as having, or being at risk for, a heart condition, disease, or disorder. Introduction of the composition can be according to methods described herein or known in the art. For example, the IMPACs, IMPAC conditioned compositions or the combination thereof (i.e., CIMPACT), can be administered to a subject's heart by way of direct injection delivery or catheter delivery. Introduction of IMPACs/CIMPACT can be a single occurrence or can occur more than one time over a period of time selected by the attending physician.

The time course and number of occurrences of CIMPACT implantation into a subject's heart can be dictated by monitoring generation and/or regeneration of cardiac tissue, where such methods of assessment and devisement of treatment course is within the skill of the art of an attending physician.

Cardiac tissue into which CIMPACT can be introduced includes, but is not limited to, the myocardium of the heart (including cardiac muscle fibers, connective tissue (endomysium), nerve fibers, capillaries, and lymphatics); the endocardium of the heart (including endothelium, connective tissue, and fat cells); the epicardium of the heart (including fibroelastic connective tissue, blood vessels, lymphatics, nerve fibers, fat tissue, and a mesothelial membrane consisting of squamous epithelial cells); and any additional connective tissue (including the pericardium), blood vessels, lymphatics, fat cells, progenitor cells (e.g., side-population progenitor cells), and nervous tissue found in the heart. Cardiac muscle fibers are composed of chains of contiguous heart-muscle cells (cardiomyocytes), joined end to end at intercalated disks. These disks possess two kinds of cell junctions: expanded desmosomes extending along their transverse portions, and gap junctions, the largest of which lie along their longitudinal portions. Each of the above tissues can be selected as a target site for introduction of CIMPACT, either individually or in combination with other tissues.

A determination of the need for treatment will typically be assessed by a history and physical exam consistent with the myocardial defect, disorder, or injury at issue. Subjects with an identified need of therapy include those with diagnosed damaged or degenerated heart tissue (i.e., heart tissue which exhibits a pathological condition) or which are predisposed to damaged or degenerative heart tissue. Causes of heart tissue damage and/or degeneration include, but are not limited to, chronic heart damage, chronic heart failure, damage resulting from injury or trauma, damage resulting from a cardiotoxin, damage from radiation or oxidative free radicals, damage resulting from decreased blood flow, and myocardial infarction (such as a heart attack). In one embodiment, a subject in need of treatment according to the methods described herein has been diagnosed with degenerated heart tissue resulting from a myocardial infarction or heart failure.

It should be recognized that methods disclosed herein can be practiced in conjunction with existing myocardial therapies to effectively treat or prevent disease. The methods, compositions, and devices of the invention can include concurrent or sequential treatment with non-biologic and/or biologic drugs.

The subject receiving cardiac implantation of CIMPACT according to the methods described herein will usually have been diagnosed as having, or being at risk for, a heart condition, disease, or disorder. The methods of treatment can be useful to alleviate the symptoms of a variety of disorders, such as disorders associated with aberrant cell/tissue damage, ischemic disorders, and reperfusion related disorders. For example, the methods are useful in alleviating a symptom of myocardial infarction, chronic coronary ischemia, arteriosclerosis, congestive heart failure, dilated cardiomyopathy, restenosis, coronary artery disease, heart failure, arrhythmia, angina, atherosclerosis, hypertension, myocardial hypertrophy, or a combination thereof. The methods of the invention can also be useful to prevent the symptoms of a variety of disorders, such as disorders associated with aberrant cell/tissue damage, ischemic disorders, and reperfusion related disorders. For example, the methods are useful in preventing a symptom of myocardial infarction, chronic coronary ischemia, arteriosclerosis, congestive heart failure, dilated cardiomyopathy, restenosis, coronary artery disease, heart failure, arrhythmia, angina, atherosclerosis, hypertension, myocardial hypertrophy, or a combination thereof. The condition, disease, or disorder can be diagnosed and/or monitored, typically by a physician using standard methodologies.

Alleviation of one or more symptoms of the cardiovascular condition, disease, or disorder indicates that the composition confers a clinical benefit, such as a reduction in one or more of the following symptoms: shortness of breath, fluid retention, headaches, dizzy spells, chest pain, left shoulder or arm pain, and ventricular dysfunction. One would understand that a reduction of one more of the symptoms need not be 100% to provide therapeutic benefit to the subject being treated. Thus, in one embodiment, a reduction of about 50%, about 60%, about 70%, about 80%, about 90%, or more of one or more such symptoms may provide sufficient therapeutic relief to a patient.

With respect to methods of prevention, one of skill in the art would understand that prevention does not necessarily mean that a patient never experiences cardiac damage. Rather, prevention includes, but is not limited to, delay of onset of one or more symptoms compared to a lack of treatment. In one non-limiting example, a patient who has a family history of fatal heart attacks by 50 years of age may experience one or more symptoms described herein, but not experience a fatal heart attack or may experience a less severe heart attack compared to lack of treatment.

Cardiac cell/tissue damage is characterized, in part, by a loss of one or more cellular functions characteristic of the cardiac cell type which can lead to eventual cell death. For example, cell damage to a cardiomyocyte results in the loss of contractile function of the cell resulting in a loss of ventricular function of the heart tissue. An ischemic or reperfusion related injury results in tissue necrosis and scar formation. Injured myocardial tissue is defined for example by necrosis, scarring, or yellow softening of the myocardial tissue. Injured myocardial tissue leads to one or more of several mechanical complications of the heart, such as ventricular dysfunction, decreased forward cardiac output, as well as inflammation of the lining around the heart (i.e., pericarditis). Accordingly, regenerating injured myocardial tissue according to the methods described herein can result in histological and functional restoration of the tissue.

In embodiments, the methods described herein promote generation and/or regeneration of heart tissue, and/or promote endogenous myocardial regeneration of heart tissue in a subject. Promoting generation of heart tissue generally includes, but is not limited to, activating, enhancing, facilitating, increasing, inducing, initiating, or stimulating the growth and/or proliferation of heart tissue, as well as activating, enhancing, facilitating, increasing, inducing, initiating, or stimulating the differentiation, growth, and/or proliferation of heart tissue cells. Thus, the methods include, for example, initiation of heart tissue generation, as well as facilitation or enhancement of heart tissue generation already in progress. Differentiation is generally understood as the cellular process by which cells become structurally and functionally specialized during development. Proliferation and growth, as used herein, generally refer to an increase in mass, volume, and/or thickness of heart tissue, as well as an increase in diameter, mass, or number of heart tissue cells. The term generation is understood to include the generation of new heart tissue and the regeneration of heart tissue where heart tissue previously existed.

Generation of new heart tissue and regeneration of heart tissue, resultant from the therapeutic methods embodied herein, can be detected and/or measured using conventional procedures in the art. Such procedures include, but are not limited to, Western blotting for heart-specific proteins, electron microscopy in conjunction with morphometry, simple assays to measure rate of cell proliferation (including trypan blue staining, the Cell Titer-Blue cell viability assay from Promega (Madison, Wis.), the MTT cell proliferation assay from American Type Culture Collection (ATCC), differential staining with fluorescein diacetate and ethidium bromide/propidium iodide, estimation of ATP levels, flow-cytometry assays, etc.), and any of the methods, molecular procedures, and assays disclosed herein.

IMPACs can be isolated from bone, purified, and cultured as described as described in US 2016/0152952 incorporated herein by reference in its entirety. Additional art-recognized methods of isolating, culturing, and differentiating stems cells are generally known in the art (see, e.g., Lanza et al., eds. (2004) Handbook of Stem Cells, Academic Press, ISBN 0124366430; Lanza et al., eds. (2005) Essentials of Stem Cell Biology, Academic Press, ISBN 0120884429; Saltzman (2004) Tissue Engineering: Engineering Principles for the Design of Replacement Organs and Tissues, Oxford ISBN 019514130X; Vunjak-Novakovic and Freshney, eds. (2006) Culture of Cells for Tissue Engineering, Wiley-Liss, ISBN 0471629359; Minuth et al. (2005) Tissue Engineering: From Cell Biology to Artificial Organs, John Wiley & Sons, ISBN 3527311866). Such methods can be utilized directly or adapted for use with the CBSCs described herein.

It will be appreciated that the time between isolation, culture, expansion, and/or implantation may vary according to a particular application and/or a particular subject. Incubation (and subsequent replication and/or differentiation) of a composition containing IMPACs can be, for example, at least in part in vitro, substantially in vitro, at least in part in vivo, or substantially in vivo. Determination of optimal culture time may be empirically determined.

IMPACs can be derived from bone of the same or different species as the transplant recipient. For example, IMPACs can be derived from an animal, including but not limited to, mammals, reptiles, and avians such as, for example, horses, cows, dogs, cats, sheep, pigs, chickens, and humans. It is also contemplated that autologous IMPACs may be obtained from the subject, into which the IMPACs are re-introduced. Such autologous IMPACs may be expanded and/or transformed before re-introduction to the host.

IMPACs may be selected and prepared for transplantation. In one aspect, therapeutic IMPACs are expanded ex vivo (or in vitro) using, for example, methods used to culture IMPACs as described in the examples section which follows. Alternatively, these cells can be expanded in vivo (i.e., after implantation). These cells can also be used for future transplantation procedures. The screened and isolated cells may, optionally, be further enriched for IMPACs prior to transplantation. Methods to select for stem cells, are well known in the art (e.g., MoFlow Cell Sorter). Methods as described herein and in US 2016/0152952, incorporated herein by reference in its entirety, are used. Alternatively, a sample of the IMPAC rich culture can be implanted without further enrichment.

Isolated IMPACs can optionally be transformed with a heterologous nucleic acid so as to express a bioactive molecule or heterologous protein or to overexpress an endogenous protein. Transformation of stem cells, including IMPACs, can be conducted using conventional methods in the art. In one non-limiting example, IMPACs may be genetically modified to expresses a fluorescent protein marker (e.g., GFP, eGFP, BFP, CFP, YFP, RFP, etc.). Marker protein expression can be especially useful in implantation scenarios, as described herein, so as to monitor IMPAC placement, retention, and replication in target tissue. As another example, IMPACs may be transfected with one or more genetic sequences that are capable of reducing or eliminating an immune response in the host.

In certain embodiments, a method for enhancing cardiac function in a subject in need thereof, comprises introducing a composition comprising IMPACs, IMPAC conditioned compositions or the combination thereof, into the heart of a subject. In certain embodiments, the IMPAC conditioned compositions are administered directly into the heart or systemically. In certain embodiments, IMPACs, IMPAC conditioned compositions or the combination thereof, are directly introduced into, or contacted with, cardiac tissue and/or cells. Introduction to the tissues or cells of a subject may occur ex vivo or in vivo. In one embodiment, compositions containing isolated cells are directly implanted into cardiac tissue of the subject, in vivo.

Improving or enhancing cardiac function generally refers to improving, enhancing, augmenting, facilitating or increasing the performance, operation, or function of the heart and/or circulatory system of a subject. Improving or enhancing cardiac function may also refer to an improvement in one or more of the following symptoms: chest pain (typically radiating to the left arm or left side of the neck), shortness of breath, nausea, vomiting, palpitations, sweating, and anxiety. The amount of cells introduced into the heart tissue of the subject can be that amount sufficient to forming endothelial cells, smooth muscle cells and/cardiomyocytes. An improvement in cardiac function may be readily assessed and determined based on known procedures including, but limited to, an electrocardiogram (ECG), echocardiography, measuring volumetric ejection fraction using magnetic resonance imaging (MRI) and/or one or more blood tests. The most often used markers for blood tests are the creatine kinase-MB (CK-MB) fraction and the troponin levels.

Introduction of cell-containing compositions can occur as a single event or over a time course of treatment. For example, compositions can be administered daily, weekly, bi-weekly, or monthly. For treatment of acute conditions, the time course of treatment generally will be at least several days. Certain conditions may extend treatment from several days to several weeks. For example, treatment could extend over one week, two weeks, or three weeks. For chronic conditions or preventative treatments, treatment regimens may extend from several weeks to several months or even a year or more.

### Administration of Immune Modulatory Paracrine Acting Cells (IMPACs)

The IMPACs can be administered by any appropriate route. In various embodiments, the IMPACs are systemically administered, e.g., intravenously, intra-arterially, or administered directly to the tissue of interest for treatment or repair (e.g., CIMPACT).

In some embodiments, the IMPACs are administered locally, e.g., directly to cardiac tissue. As appropriate, the IMPACs can be engrafted or transplanted into and/or around the tissue of interest, e.g., cardiac tissues. When engrafted or transplanted into and/or in the vicinity of one or more tissues of interest (e.g., cardiac tissues), the IMPACs are administered within or within sufficient proximity of inflamed or damaged lesions in tissue to mitigate and/or reverse of damage and/or destruction of the tissue. For example, the IMPACs are engrafted or transplanted into or within sufficient proximity to the tissue of interest to prevent, reduce or inhibit damage and/or destruction to the tissues.

As appropriate, injections of IMPACs can be done after local anesthetics (e.g., lidocaine, bupivacaine) have been administered. It is also possible to inject the IMPACs in conjunction with local anesthetics added to the cell suspension. Injections can also be made with the subject under general anesthesia with or without the use of local anesthetic agents (e.g., lidocaine). In one aspect, the cells may be transplanted along with a carrier material, such as collagen or fibrin glue or other scaffold materials. Such materials may improve cell retention and integration after implantation. Exemplary materials and methods for employing them are known in the art and are contemplated herein (see, e.g., Saltzman (2004) Tissue Engineering: Engineering Principles for the Design of Replacement Organs and Tissues, Oxford ISBN 019514130X; Vunjak-Novakovic and Freshney, eds. (2006) Culture of Cells for Tissue Engineering, Wiley-Liss, ISBN 0471629359; and Minuth et al. (2005) Tissue Engineering: From Cell Biology to Artificial Organs, John Wiley & Sons, ISBN 3527311866).

In certain embodiments, engraftment or transplantation of the IMPACs can be facilitated using a matrix or caged depot. For example, the IMPACs can be engrafted or transplanted in a “caged cell” delivery device wherein the cells are integrated into a biocompatible and/or biologically inert matrix (e.g. a hydrogel or other polymer or any device) that restricts cell movement while allowing the cells to remain viable. Synthetic extracellular matrix and other biocompatible vehicles for delivery, retention, growth, and differentiation of IMPACs are known in the art and find use in the present methods. See, e.g., Prestwich, J Control Release. 2011 Apr. 14, PMID 21513749; Perale, et al., Int J Artif Organs. (2011) 34(3):295-303; Suri, et al., Tissue Eng Part A. (2010) 16(5):1703-16; Khetan, et al., J Vis Exp. (2009) October 26; (32). pii: 1590; Salinas, et al., J Dent Res. (2009) 88(8):681-92; Schmidt, et al., J Biomed Mater Res A. (2008) 87(4):1113-22 and Xin, et al., Biomaterials (2007) 28:316-325.

As appropriate or desired, the engrafted or transplanted IMPACs can be modified to facilitate retention of the IMPACs at the region of interest or the region of delivery. In other embodiments, the region of interest for engraftment or transplantation of the cells is modified in order to facilitate retention of the IMPACs at the region of interest or the region of delivery, e.g. cytokines, growth factors etc. Therapeutic cells may be implanted into a subject using conventional methods (see, for example, Orlic et al. (2001) Nature, 410(6829): 701-705). For example, cells, or compositions comprising cells, may be introduced via direct injection (e.g., intermyocardial or intercoronary injection) or catheter-based delivery (e.g., intermyocardial, intercoronary, or coronary sinus delivery). Intercoronary catheter delivery directly injects cells into heart tissue.

The amount of cells introduced into the heart tissue of the subject can be that amount sufficient to improve cardiac function, increase cardiomyocyte formation, and/or increase mitotic index of cardiomyocytes. For example, an effective amount may increase cardiomyocyte formation, increase cardiomyocyte proliferation, increase cardiomyocyte cell cycle activation, increased mitotic index of cardiomyocytes, increase myofilament density, increase borderzone wall thickness, or a combination thereof. An effective amount may form endothelial cells, smooth muscle cells, cardiomyocytes, or a combination thereof.

In some embodiments, at least about 1 million IMPACs/kg subject are administered or engrafted, e.g., at least about 2 million, 2.5 million, 3 million, 3.5 million, 4 million, 5 million, 6 million, 7 million, 8 million, 9 million or 10 million IMPACs/kg subject are administered. In varying embodiments, about 1 million to about 10 million IMPACs/kg subject, e.g., about 2 million to about 8 million IMPACs/kg are administered or grafted.

In varying embodiments, at least about 5 million IMPACs are administered or engrafted, e.g., at least about 10 million, 15 million, 20 million, 25 million, 30 million, 35 million, 40 million, 45 million, 50 million, 55 million, 60 million, 65 million, 70 million, 75 million, 80 million, 85 million, 90 million, 95 million or 100 million IMPACs are administered or engrafted. In varying embodiments, about 5 million to about 100 million IMPACs are administered or engrafted, e.g., about 10 million to about 80 million IMPACs are administered or engrafted. In varying embodiments, at least about 50 million IMPACs are administered or engrafted, e.g., at least about 100 million, 150 million, 200 million, 250 million, 300 million, 350 million, 400 million, 450 million, 500 million, 550 million, 600 million, 650 million, 700 million, 750 million, 800 million, 850 million, 900 million, 950 million or 1 billion IMPACs are administered or engrafted.

In certain embodiments, the IMPACs are administered, e.g., intravenously, at a rate of about 1 million to about 10 million cells per minute, e.g., at a rate of about 2 million to about 4 million cells per minute, e.g., at a rate of about 2.5 million to about 3.5 million cells per minute.

Treatment or prevention may involve one or multiple injections. For example, IMPACs may be administered to the subject 1, 2, 3, 4, 5, 6, 7, 8, 9, 10, or more times, as appropriate. Subsequent administrations of IMPACs may be administered systemically or locally. If administered locally, multiple injections of IMPACs may be administered to the same or different locations. Multiple injections of IMPACs can be administered daily, weekly, bi-weekly, monthly, bi-monthly, every 3, 4, 5, or 6 months, or annually, or more or less often, as needed by the subject. The frequency of administration of the IMPACs can change over a course of treatment, e.g., depending on how well the engrafted or transplanted IMPACs establish themselves at the site of administration and the responsiveness of the subject. The IMPACs may be administered multiple times over a regime course of several weeks, several months, several years, or for the remainder of the life of the subject, as needed or appropriate.

In certain embodiments, the IMPACs are administered with a IMPAC conditioned composition. The IMPAC conditioned composition can be administered, prior to, in conjunction with and after IMPAC administration. The administration of the conditioned composition can be extended until the IMPACs have differentiated into the appropriate cardiac cell, e.g. cardiac myocyte.

The total amount of cells that are envisioned for use depend upon the desired effect, patient state, and the like, and may be determined by one skilled within the art. Dosages for any one patient depends upon many factors, including the patient's species, size, body surface area, age, the particular IMPACs to be administered, sex, scheduling and route of administration, general health, and other drugs being administered concurrently.

Also provided herein is a composition comprising a population of IMPACs and a pharmaceutically acceptable carrier for treating myocardial infarction, chronic coronary ischemia, arteriosclerosis, congestive heart failure, dilated cardiomyopathy, restenosis, coronary artery disease, heart failure, arrhythmia, angina, atherosclerosis, hypertension, or myocardial hypertrophy, wherein the CBSCs are isolated from the bone or fragments thereof; are negative for one or more markers comprising: hematopoietic and lineage stem cell markers, CD34, CD117, CD11b, CD31, CD45, major histocompatibility complex (MHC) class II molecules, MHC class Ia molecules, HLA-A, B, C or co-stimulatory molecules; are positive for CD49f (integrin alpha chain 6).

Also provided herein is a composition comprising a population of IMPACs and/or a IMPAC conditioned composition and a pharmaceutically acceptable carrier for inducing cardiac regeneration, wherein the IMPACs are isolated from the bone or fragments thereof; are negative for one or more markers comprising: hematopoietic and lineage stem cell markers, CD34, CD117, CD11b, CD31, CD45, major histocompatibility complex (MHC) class II molecules, MHC class Ia molecules, HLA-A, B, C or co-stimulatory molecules; are positive for CD49f (integrin alpha chain 6). In another embodiment, the compositions increase cardiomyocyte formation, increase cardiomyocyte proliferation, increase cardiomyocyte cell cycle activation, increase mitotic index of cardiomyocytes, increase myofilament density, increase border zone wall thickness, or a combination thereof, when administered to a subject. In another embodiment, the composition treats myocardial infarction, chronic coronary ischemia, arteriosclerosis, congestive heart failure, dilated cardiomyopathy, restenosis, coronary artery disease, heart failure, arrhythmia, angina, atherosclerosis, hypertension, or myocardial hypertrophy when administered to a subject.

One would understand that one or more additional excipients, carriers, adjuvants, cells, etc., may be added to the compositions described herein.

### Methods of Monitoring

Clinical efficacy can be monitored using any method known in the art. Measurable parameters to monitor efficacy will depend on the condition being treated. For monitoring the status or improvement of one or more symptoms associated with cardiomyopathy, measurable parameters can include without limitation, auditory inspection (e.g., using a stethoscope), blood pressure, electrocardiogram (EKG), magnetic resonance imaging (MRI), changes in blood markers, and behavioral changes in the subject (e.g., appetite, the ability to eat solid foods, grooming, sociability, energy levels, increased activity levels, weight gain, exhibition of increased comfort). These parameters can be measured using any methods known in the art. In varying embodiments, the different parameters can be assigned a score. Further, the scores of two or more parameters can be combined to provide an index for the subject.

Observation of the stabilization, improvement and/or reversal of one or more symptoms or parameters by a measurable amount indicates that the treatment or prevention regime is efficacious. Observation of the progression, increase or exacerbation of one or more symptoms indicates that the treatment or prevention regime is not efficacious. For example, in the case of cardiomyopathy, observation of the improvement of cardiac function (e.g., blood pressure in appropriate range, stable heart rhythm or reduction or absence of arrhythmias, changes in blood markers, and/or behavioral changes in the subject (e.g., increased appetite, the ability to eat solid foods, improved/increased grooming, improved/increased sociability, increased energy levels, improved/increased activity levels, weight gain and/or stabilization, exhibition of increased comfort) after one or more co-administrations of stem cells with an agent indicates that the treatment or prevention regime is efficacious. Likewise, observation of reduction or decline of cardiac function (e.g., blood pressure in appropriate range, unstable heart rhythm or continued presence or increased arrhythmias, changes in blood markers, and/or behavioral changes in the subject (e.g., decreased appetite, the inability to eat solid foods, decreased grooming, decreased sociability, decreased energy levels, decreased activity levels, weight loss, exhibition of increased discomfort) after one or more co-administrations of stem cells with an agent indicates that the treatment or prevention regime is not efficacious.

In certain embodiments, the monitoring methods can entail determining a baseline value, for example, of a measurable biomarker, imaging or disease parameter in a subject before administering a dosage of the IMPACs and/or IMPAC conditioned composition described herein, and comparing this with a value for the same measurable biomarker or parameter after a course of treatment.

In other methods, a control value (i.e., a mean and standard deviation) of the measurable biomarker or parameter is determined for a control population. In certain embodiments, the individuals in the control population have not received prior treatment and do not have the disease condition subject to treatment (e.g., cardiomyopathy), nor are at risk of developing the disease condition subject to treatment (e.g., cardiomyopathy). In such cases, if the value of the measurable biomarker or clinical parameter approaches the control value, then treatment is considered efficacious. In other embodiments, the individuals in the control population have not received prior treatment and have been diagnosed with the disease condition subject to treatment (e.g., cardiomyopathy). In such cases, if the value of the measurable biomarker or clinical parameter approaches the control value, then treatment is considered inefficacious.

In other methods, a subject who is not presently receiving treatment but has undergone a previous course of treatment is monitored for one or more of the biomarkers or clinical parameters to determine whether a resumption of treatment is required. The measured value of one or more of the biomarkers or clinical parameters in the subject can be compared with a value previously achieved in the subject after a previous course of treatment. Alternatively, the value measured in the subject can be compared with a control value (mean plus standard deviation) determined in population of subjects after undergoing a course of treatment. Alternatively, the measured value in the subject can be compared with a control value in populations of prophylactically treated subjects who remain free of symptoms of disease, or populations of therapeutically treated subjects who show amelioration of disease characteristics. In such cases, if the value of the measurable biomarker or clinical parameter approaches the control value, then treatment is considered efficacious and need not be resumed. In all of these cases, a significant difference relative to the control level (i.e., more than a standard deviation) is an indicator that treatment should be resumed in the subject.

In certain embodiments, immunohistochemical approaches are utilized to detect formation of endothelial cells, smooth muscle cells, and cardiomyocytes in the infarcted hearts. Cardiac function enhancement can also be detected with magnetic resonance imaging (MRI).

### EXAMPLES

### Example 1: Human Bone is a Source of IMPACs with Ability to Control Cellular Homeostasis

A cell derived from the bone stroma, termed herein Immune Modulatory Paracrine Acting Cells (IMPACs), was developed, that has the unique ability to thrive in hostile environments where it can secrete paracrine factors and exosomes that modify the local environment to reduce inflammation, rescue stressed muscle cells, and reduce injury induced scar formation. Allogeneic treatment of the IMPACs into border zone of mouse myocardial infarction (MI) and pig ischemia/reperfusion MI (MI/R) induced reduction in ventricular remodeling and improved cardiac functions. In all these studies one of the most significant result was reduced infarct size after IMPACs treatment. There was evidence of newly formed vasculatures and cardiac myocytes; however, the number of those myocytes were very few and did not appear to contribute to the functional improvements. Instead, the IMPACs secreted paracrine factors that support the reparative effects and are the likely mechanisms for the post MI and MI/R improvements. Therefore, these paracrine mechanisms need to be elucidated to develop future therapies. Preclinical studies with IMPACs derived from human bone biopsies are required to be able to have innovative insights into the bases of IMPAC-mediated post MI repair. Therefore, in the present study, newly identified IMPACs were characterized to confirm their efficacy for treating myocardial injuries using human to mouse xenografic MI/R model by augmenting wound healing processes. Post injury inflammation is a pre-requisite for healing; however, over-recruitment and activation of infiltrating immune cells exert potent cytotoxic effects and cause death of cardiac myocyte that survived the initial infarction insult resulting expansion of the infarct. Therefore, a cell type that can modify inflammatory response, immune cell phenotypic shift will augment the benefits related to cell therapy. Indeed, the analysis of IMPACs exosomal micro RNAs (miRNAs) indicated a unique combination of counter acting miRNAs in the area of inflammation, survival, and growth that can provide “a three-dimensional control” of cellular homeostasis much needed after cardiac injury. The IMPACs can maintain a delicate balance by bi-directionally modifying cellular behaviors that can augment wound healing processes. This cellular homeostasis may be the source of stem cell induced paracrine mechanisms and reparative effects.

### Results:

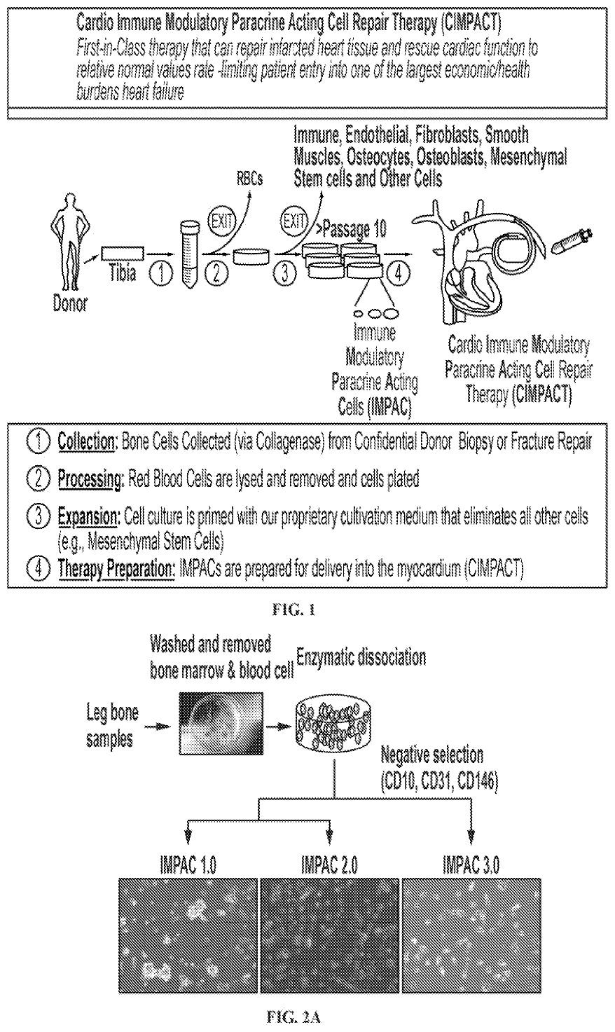

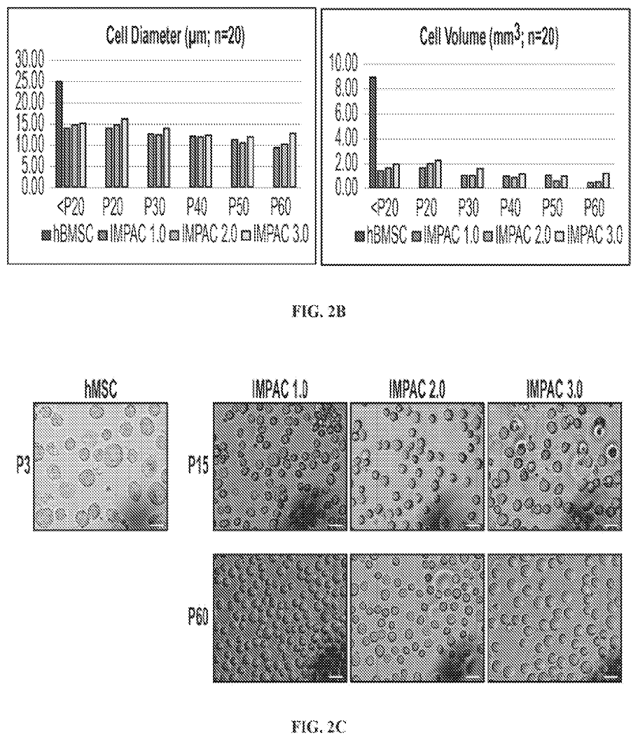

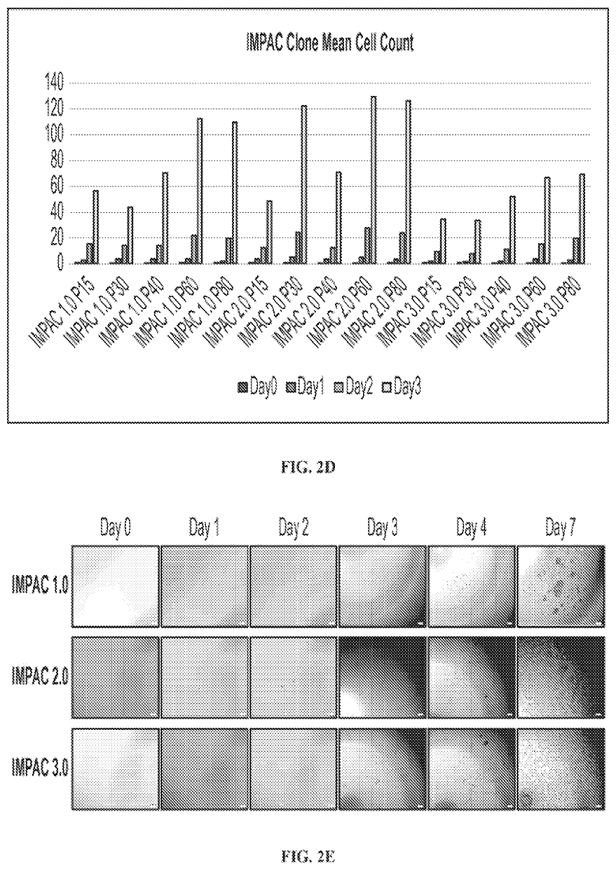

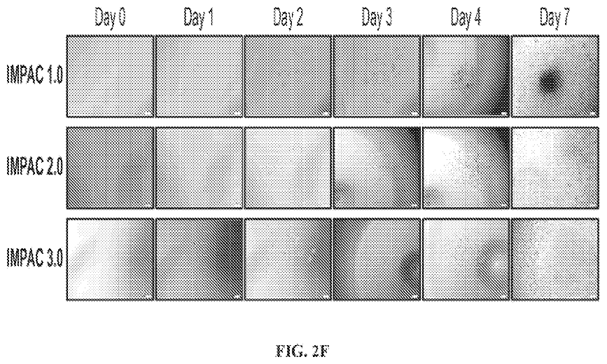

Human Bone-derived IMPACs have stem cell like phenotype and growth kinetics in-vitro (FIGS. 2A-2F, Tables 1A, 1B and 2): IMPACs were distinctly smaller in size when compared to human bone marrow stromal cells (hBMSC) and had tendency to become even smaller as passage number increased. The smallest mean cell volume IMPACs were measured at passage 60 was 0.48 mm³ which was nearly 20-fold smaller than the mean hBMSC volume (FIG. 2B). IMPACs were highly clonogenic and grew without senescence in culture up to passage 100. The mean doubling times (hour) measured between zero to 72 h of the culture was 13.00 hours at a lower passage (<20) and 10.82, and at the higher passage (P80) respectively (FIGS. 2C-2F). These data indicate that the IMPACs in culture are immortalized cells and have tendency to become smaller and proliferate at a faster rate as the passage number increases.

TABLE 1-A
Diameter of IMPAC Phenotypes

| | Cell Diameter (μm) | | | |
|---|---|---|---|---|
| Passage# | hBMSC | IMPAC 1.0 | IMPAC 2.0 | IMPAC 3.0 |
| <P20 | 25.17 | 13.86 | 14.58 | 15.29 |
| P20 | NA | 14.22 | 15.00 | 16.17 |
| P30 | NA | 12.68 | 12.30 | 13.91 |
| P40 | NA | 11.93 | 11.83 | 12.37 |
| P50 | NA | 11.33 | 10.61 | 12.17 |
| P60 | NA | 9.41 | 10.19 | 12.88 |

TABLE 1-B
Volume of IMPAC Phenotypes

| | Cell Volume (mm³) | | | |
|---|---|---|---|---|
| Passage# | hBMSC | IMPAC 1.0 | IMPAC 2.0 | IMPAC 3.0 |
| <P20 | 8.90 | 1.48 | 1.73 | 1.81 |
| P20 | NA | 1.63 | 1.96 | 2.26 |
| P30 | NA | 1.12 | 1.02 | 1.51 |
| P40 | NA | 0.96 | 0.93 | 1.08 |
| P50 | NA | 1.06 | 0.66 | 0.97 |
| P60 | NA | 0.48 | 0.58 | 1.17 |

TABLE 2
IMPAC Clone Mean Cell Count and Doubling Hours

| Cell Type | Passage# | n | Day 0 | Day 1 | Day 2 | Day 3 | Doubling (h) |
|---|---|---|---|---|---|---|---|
| IMPAC 1.0 | 15 | 8 | 1 | 4.38 | 15.75 | 58.00 | 13.20 |
| IMPAC 2.0 | 15 | 7 | 1 | 3.29 | 12.86 | 48.86 | 13.00 |
| IMPAC 3.0 | 15 | 8 | 1 | 2.63 | 9.13 | 34.75 | 14.69 |
| IMPAC 1.0 | 30 | 10 | 1 | 4.00 | 13.50 | 44.70 | 13.63 |
| IMPAC 2.0 | 30 | 7 | 1 | 5.63 | 24.00 | 123.13 | 11.77 |
| IMPAC 3.0 | 30 | 8 | 1 | 2.11 | 8.56 | 34.22 | 16.51 |
| IMPAC 1.0 | 40 | 8 | 1 | 3.50 | 14.50 | 70.50 | 12.09 |
| IMPAC 2.0 | 40 | 8 | 1 | 3.50 | 12.63 | 70.50 | 12.05 |
| IMPAC 3.0 | 40 | 8 | 1 | 3.00 | 10.50 | 52.38 | 13.49 |
| IMPAC 1.0 | 60 | 10 | 1 | 3.90 | 21.40 | 114.10 | 11.03 |
| IMPAC 2.0 | 60 | 9 | 1 | 4.78 | 27.22 | 131.56 | 11.78 |
| IMPAC 3.0 | 60 | 10 | 1 | 3.60 | 15.70 | 68.50 | 12.21 |
| IMPAC 1.0 | 80 | 8 | 1 | 2.88 | 20.29 | 109.25 | 11.53 |
| IMPAC 2.0 | 80 | 8 | 1 | 3.75 | 25.25 | 128.50 | 10.82 |
| IMPAC 3.0 | 80 | 6 | 1 | 3.67 | 18.67 | 69.50 | 12.51 |

Note:
Doubling time was calculated between Day 0 and 3 using online calculator Roth V. 2006 Doubling Time Computing.

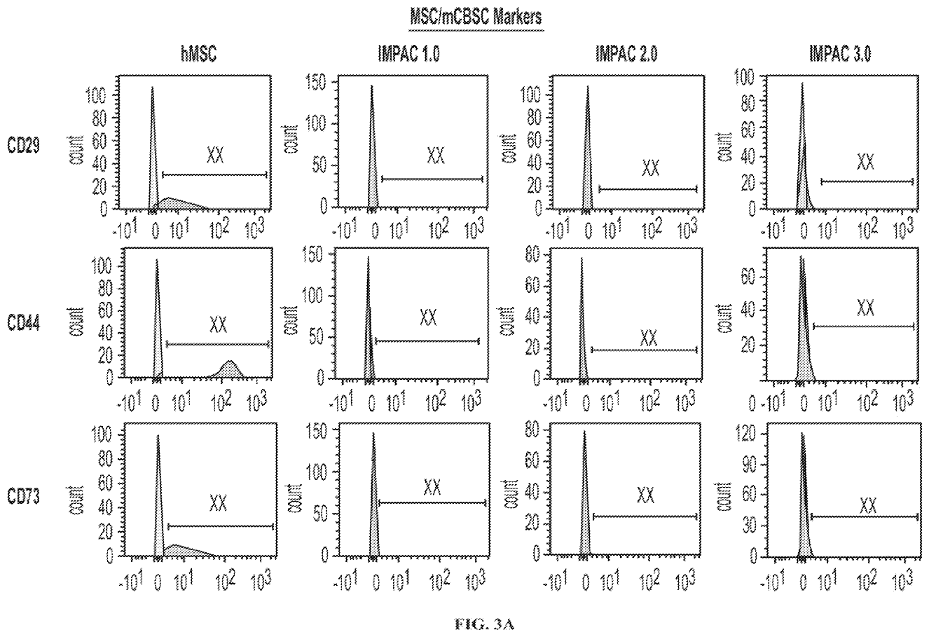

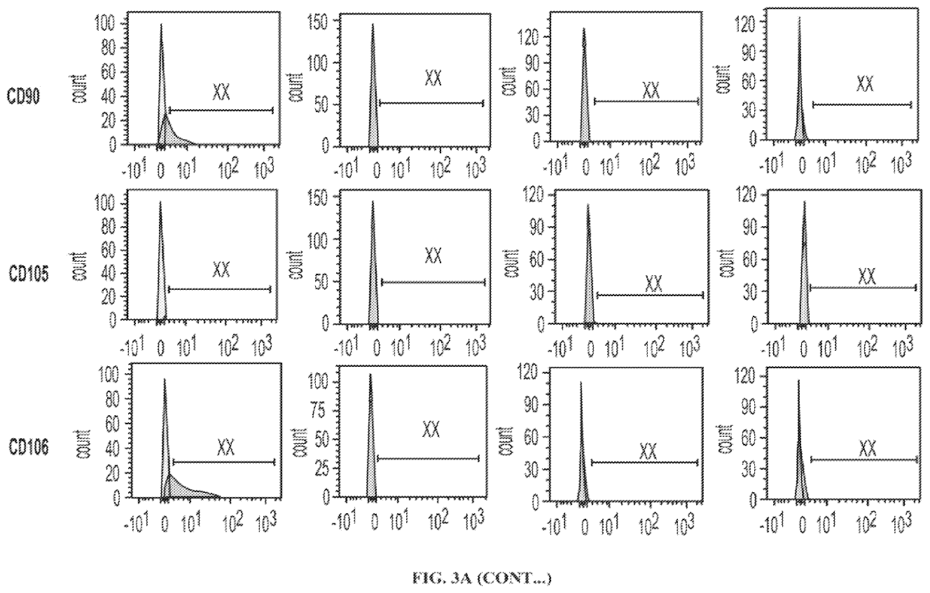

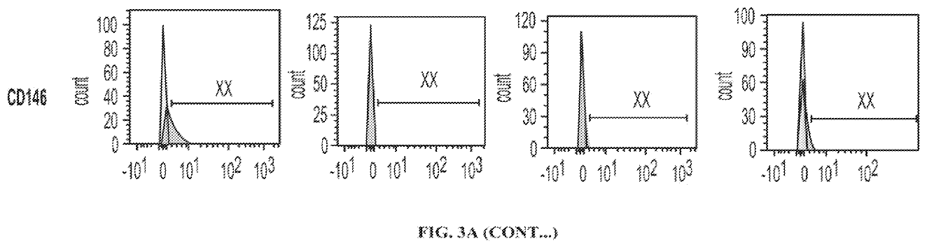

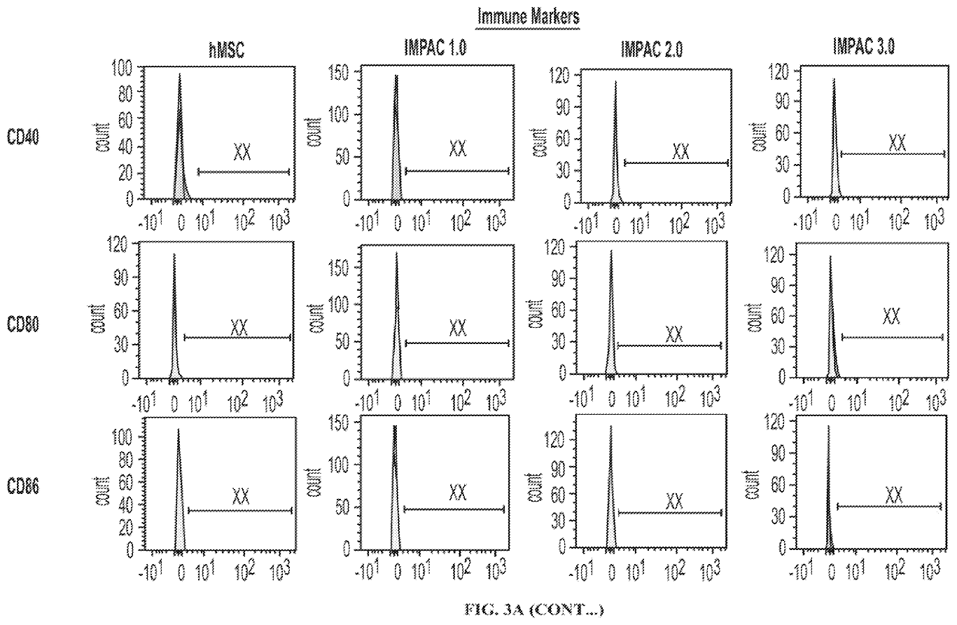

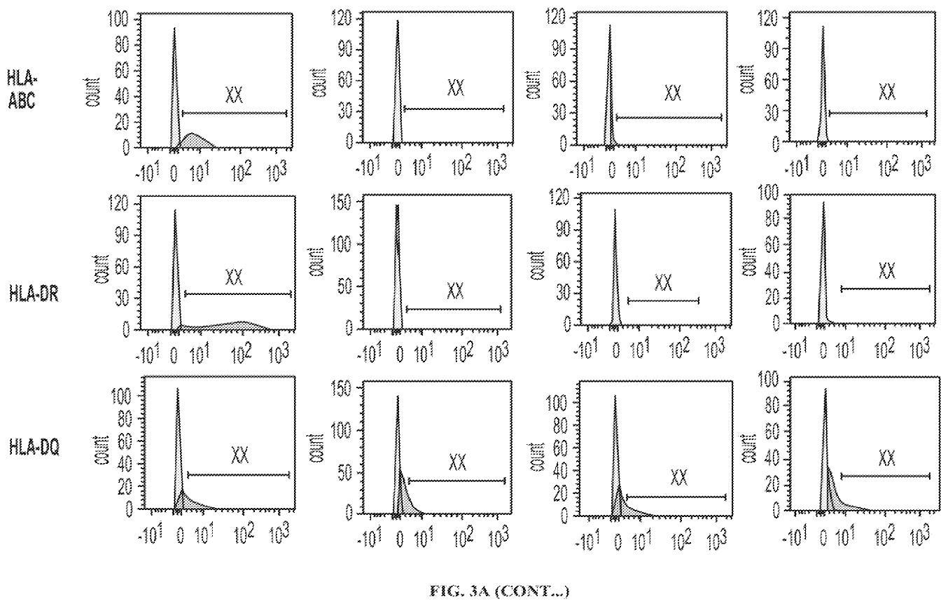

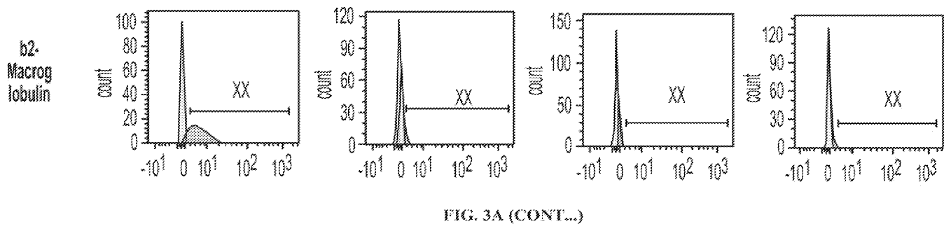

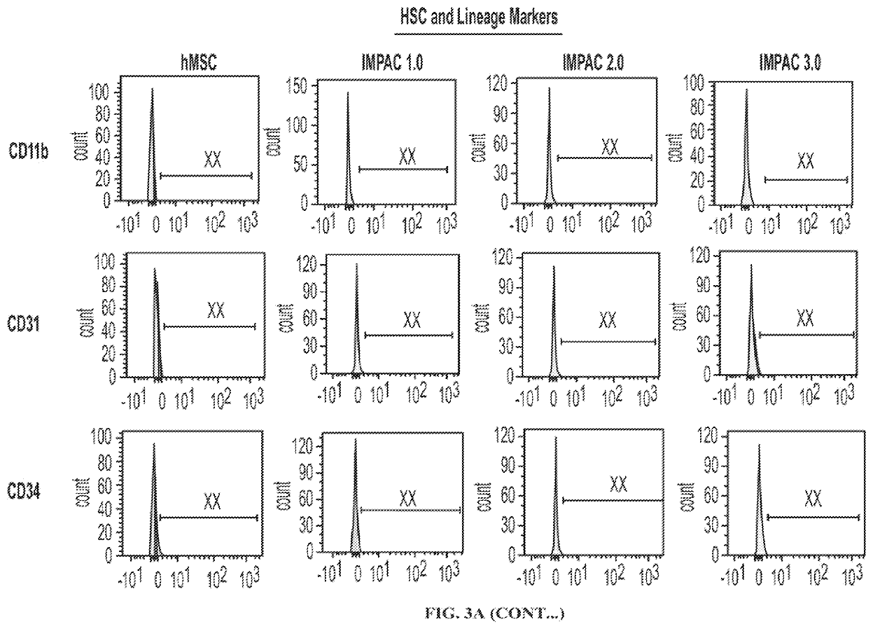

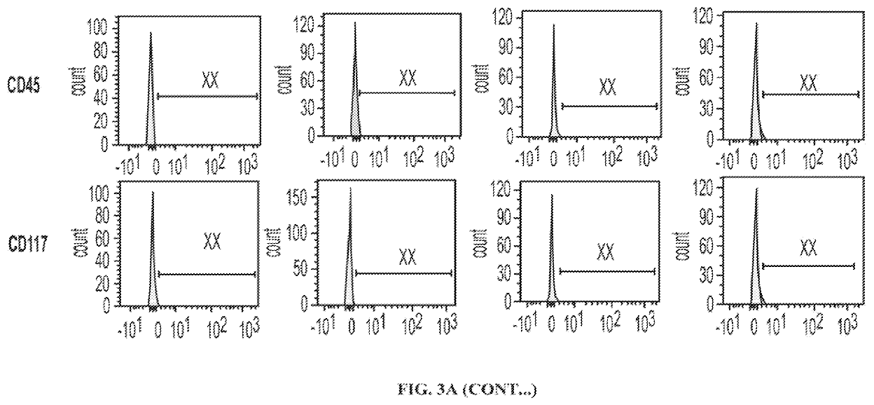

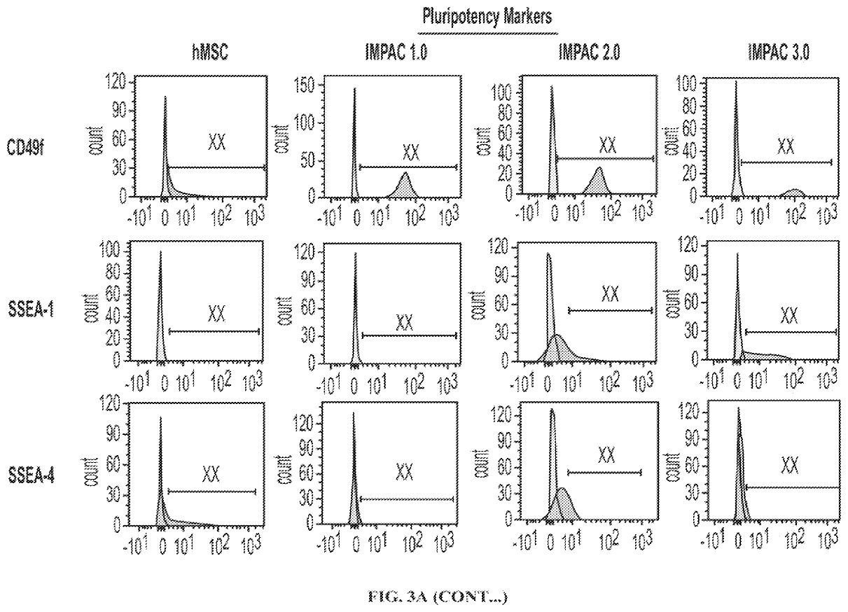

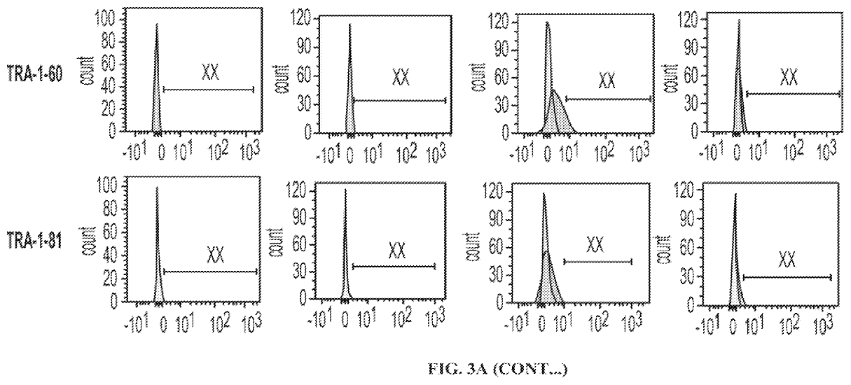

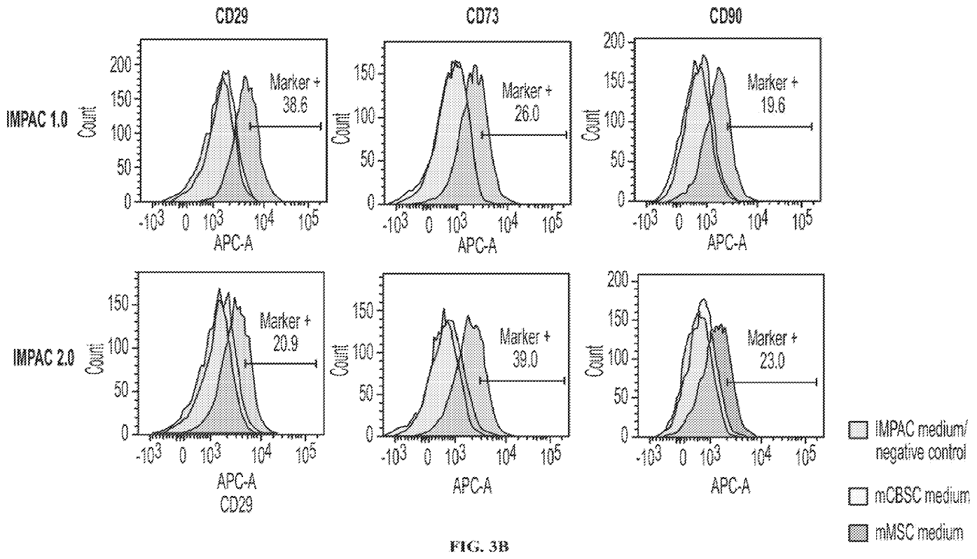

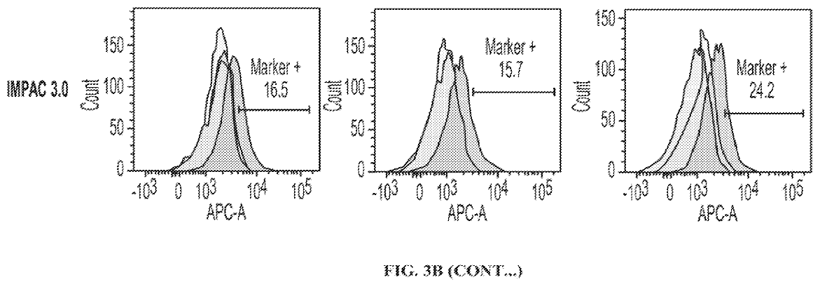

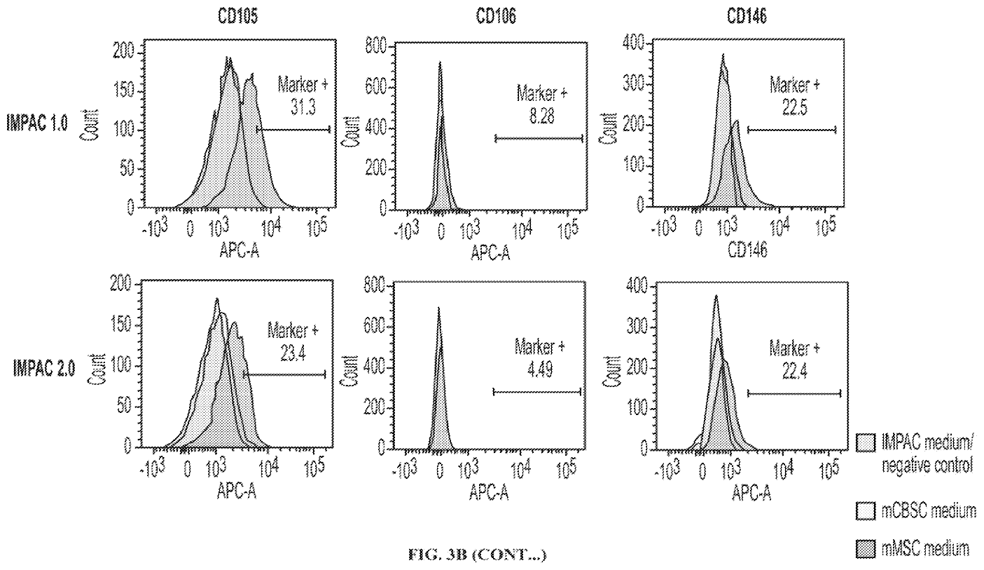

Human Bone-derived IMPACs have unique surface marker characteristics (FIGS. 3A, 3B, Table 3): To determine surface antigen marker characteristics of IMPACs, a total of 242 human, 176 mouse and 6 pig cell-surface antigen markers were screened on IMPACs (FIG. 3A and Table 3). It was found that the IMPAC phenotypes expressed very few surface markers and were different versus the mCBSCs. IMPACs were negative for hematopoietic and lineage stem cell markers including CD34 and CD117, CD11b, CD31, and CD45. Additionally, IMPACs were negative for the major histocompatibility complex (MHC) class II molecules (except HLA-DQ), MHC class Ia molecules, HLA-A, B, and C (“self-antigen”) and co-stimulatory molecules indicating IMPACs are neither professional or non-professional antigen presenting cells (3) (FIG. 2A). The most distinctive marker found in IMPACs (100%) was CD49f (integrin alpha chain 6) which is known to enhance self-renewal, multipotency and maintain stemness (4, 5). Interestingly, IMPAC phenotypes expressed surface markers known to be found in embryonic CBSCs during their differentiation including SSEA-1, SSEA-4, TRA-1-60, and TRA-1-81 (40%, 33.6%, 6.39%, and 3.25% respectively). These results show that the IMPACs resemble primitive cells that resides deeper into the bone expressing hESC markers.

TABLE 3
CBSC Special Diversity in Surface Markers

| Marker | hBMSC | IMPAC 1.0 | IMPAC 2.0 | IMPAC 3.0 | mCBSC | sCBSC |
|---|---|---|---|---|---|---|
| CD11b | − | − | − | − | − | − |
| CD14 | − | − | − | − | − | |
| CD19 | − | − | − | − | − | |
| CD31 | − | − | − | − | − | |
| CD34 | − | − | − | − | − | − |
| CD45 | − | − | − | − | − | |
| CD29 | ++++ | − | − | − | ++++ | +++++ |
| CD44 | +++++ | − | − | − | +++++ | +++++ |
| CD73 | +++++ | − | − | − | +++++ | |
| CD90/CD90.2 | +++ | − | − | − | − | |
| CD105 | ++++ | − | − | − | +++++ | − |
| CD106 | + | − | − | − | +++++ | + |
| SSEA-1 | − | − | ++ | ++++ | +++ | |
| SSEA-4 | ++ | − | ++ | − | ++ | |
| CD49f | +++ | +++++ | +++++ | +++++ | ++++ | |

−: <5%;
+: <20%;
++: <40%;
+++: <60%;
++++: <80%;
+++++: <100%

The Mesenchymal and Tissue Stem Cell Committee of the International Society for Cellular Therapy proposed minimum criteria to define human MSC (hMSC) are;
1) hMSC must be plastic-adherent when maintained in standard culture conditions;
2) Express CD105, CD73 and CD90;
3) Lack surface expression of CD45, CD34, CD14 or CD11b, CD79a or CD19 and HLA-DR.

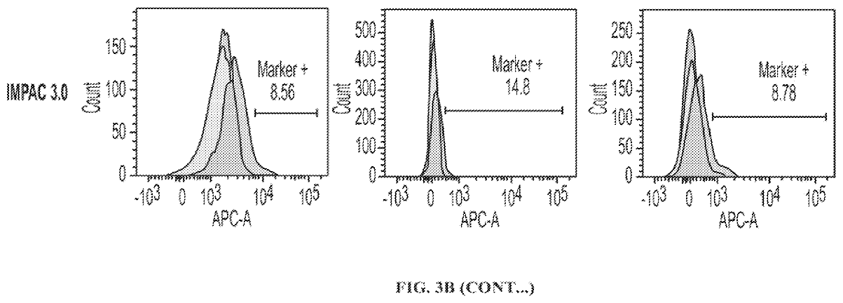

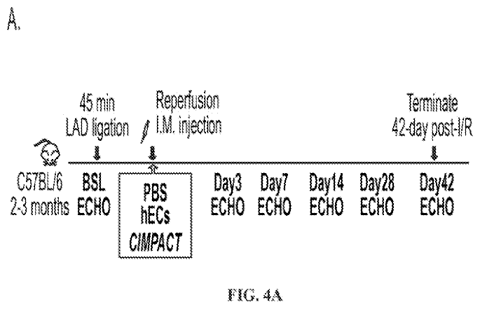

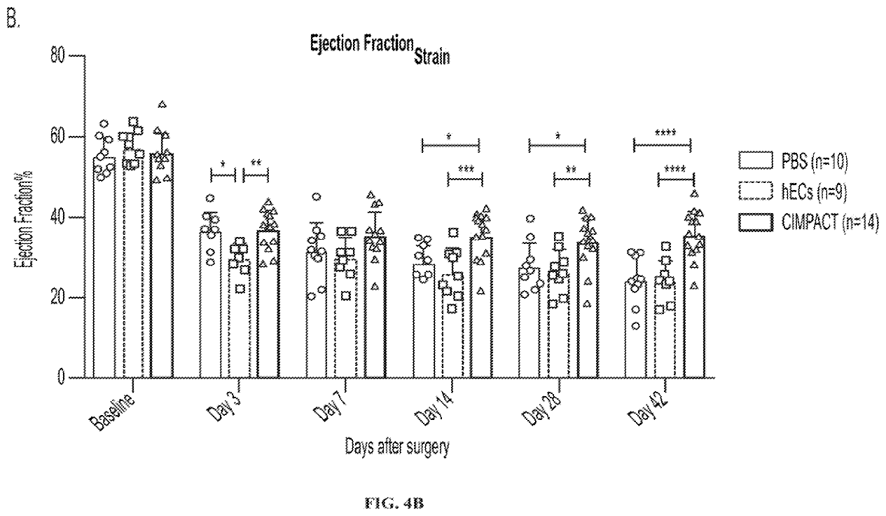

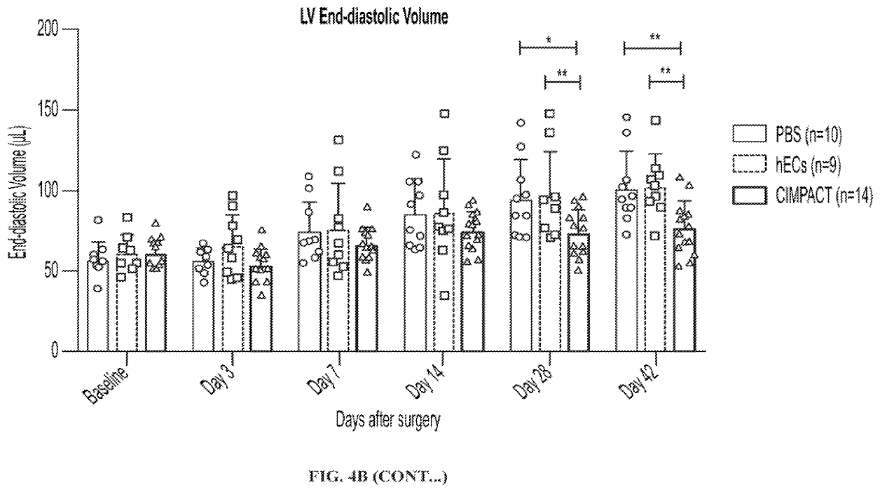

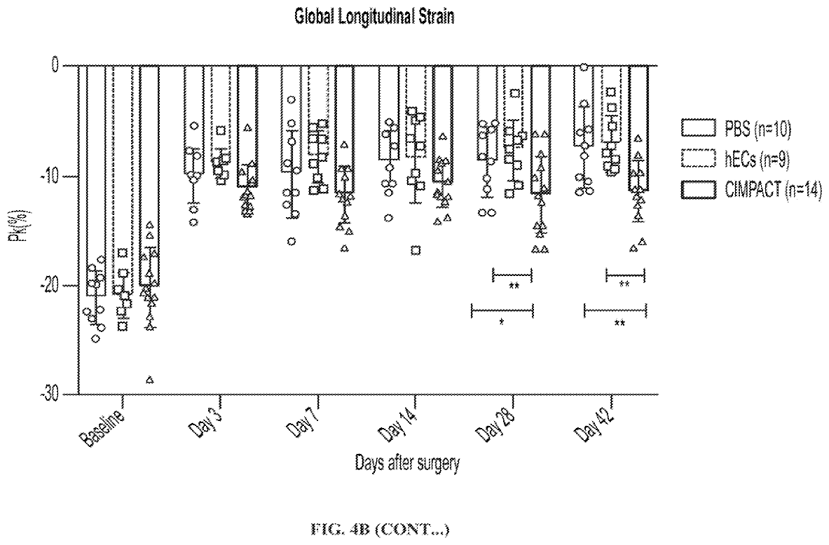

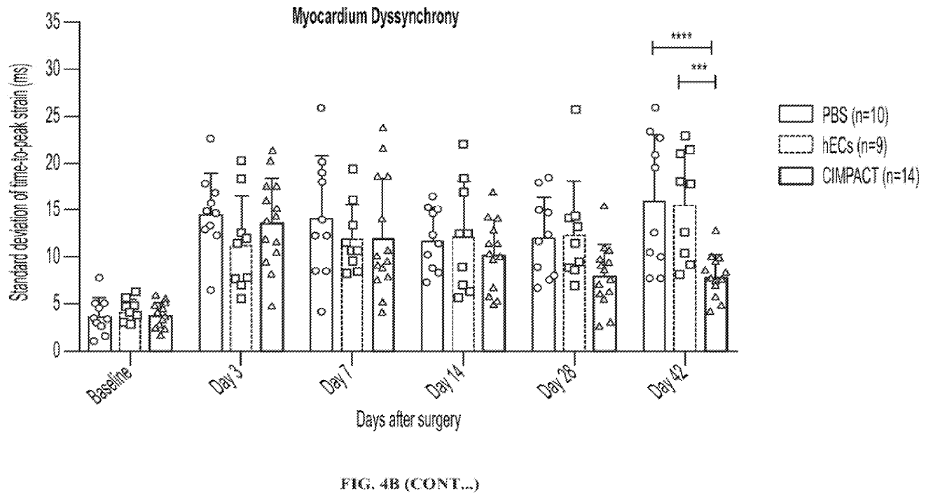

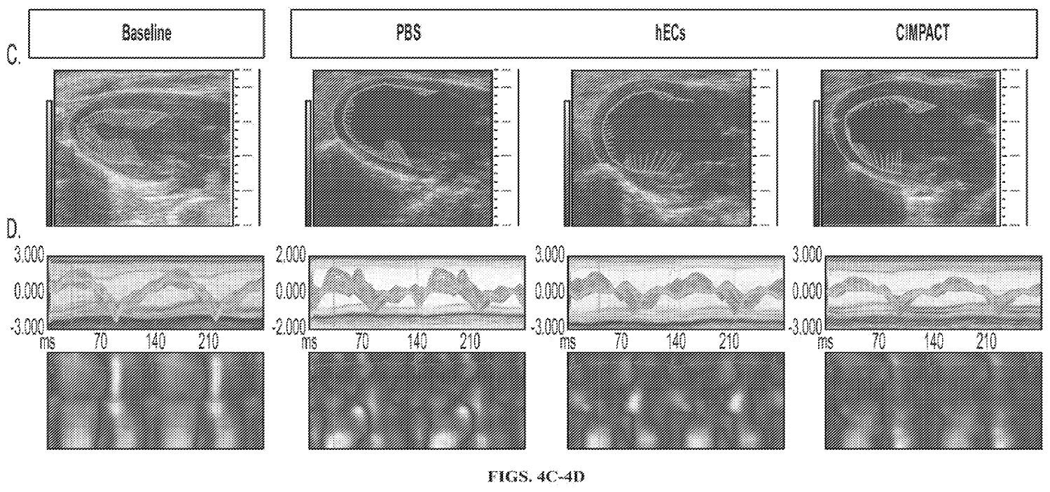

IMPACXenograft reduced myocardial remodeling and improved cardiac functions (FIGS. 4A, 4B): The reparative effect of IMPACs treatment post-I/R was investigated in a mouse model. Wild-type female C57BL/6 mice were used in the in vivo study. Cardiac function and structural remodeling were traced by Echocardiography (ECHO) at baseline, 3-day, 7-day, 14-day, 28-day and 42-day post I/R surgery (FIG. 4A). For long-term functional analysis, in order to eliminate the bias caused by failure of surgery, mice that had an Ejection Fraction (EF) higher than 45% 3-day post I/R were considered as failure of surgery and removed from the statistics for the long-term functional analysis. Scar size of the excluded mice were analyzed at terminal to confirm the absent of actual ischemic injury (FIG. 10A). Moreover, mice randomly assigned to short-term study were terminated 3-day post-I/R for histology analysis, scar size of mice with different treatments presented no difference at 3-day post-I/R (FIG. 10B), providing evidence that there was no difference of initial myocardial injury and no significant reparative effect of any treatments at early timepoints. Two-dimensional speckle tracking ECHO analysis showed that the EF 3-day post-I/R had no difference between mice treated with CIMPACT, GFP+ CIMPACT and PBS. However, hECs treated mice have lower EF at day 3. EF significantly declined from early time points to 42 days post-I/R in hECs and PBS treated mice but preserved in s treated groups (FIG. 4B). Global averages of LV endocardial longitudinal strain, which provides evidence that the changes in length as proportion to baseline length, were also preserved in mice treated with CIMPACT and GFP+ CIMPACT. Radial strain also had a trend of less reduction in mice treated with CIMPACT after I/R. Moreover, standard deviation of time-to-peak strain which suggests myocardium dyssynchrony showed a significant increase in mice treated with hECs and PBS at 42-day post-I/R. Representative images showing vector diagram of long-axis B-mode at baseline and 42-day post-I/R were presented in FIG. 4C. Normal hearts have uniform, synchronous contractions and relaxations. However, this could be impaired by I/R injury with hypokinesis of the infarcted wall and chamber dilation. Mice received hECs and PBS treatment showed more severe chamber dilation, hypokinesis and thinning of anterior wall compared to mice who received CIMPACT. FIG. 4D graphed the dynamic changes of the endocardial radial velocity and suggested reduction and dyssynchrony of radial velocity in mice treated with hECs or PBS. FIG. 4E are representative 3-dimensional wall velocity diagrams and which showed a dramatic reduction of wall velocity across endocardium of the infarcted anterior wall in hECs and PBS treated mice. This reduction is largely preserved in CIMPACT treated mice. FIG. 4F showed the representative endocardial longitudinal strain waveforms. Points at the bottom of each waveform shows the time-to-peak value of each segments of the heart including base, mid and apex from both anterior and posterior wall. In normal heart, the time-to-peak of each segments were highly consistent, the longitudinal strain showed smooth waveforms. However, mice received hECs and PBS treatment post I/R presented a dramatic dyssynchrony of different segments of the heart with a dramatic delay in contraction in some segments, which lead to an increase of standard deviation of longitudinal time-to-peak in FIG. 4B. Overall, these data provide evidence that CIMPACT treatment post-I/R largely preserved the contractility of the heart with less reduction in ejection fraction and preserved myocardium synchronous compared to mice treated with hECs and PBS.

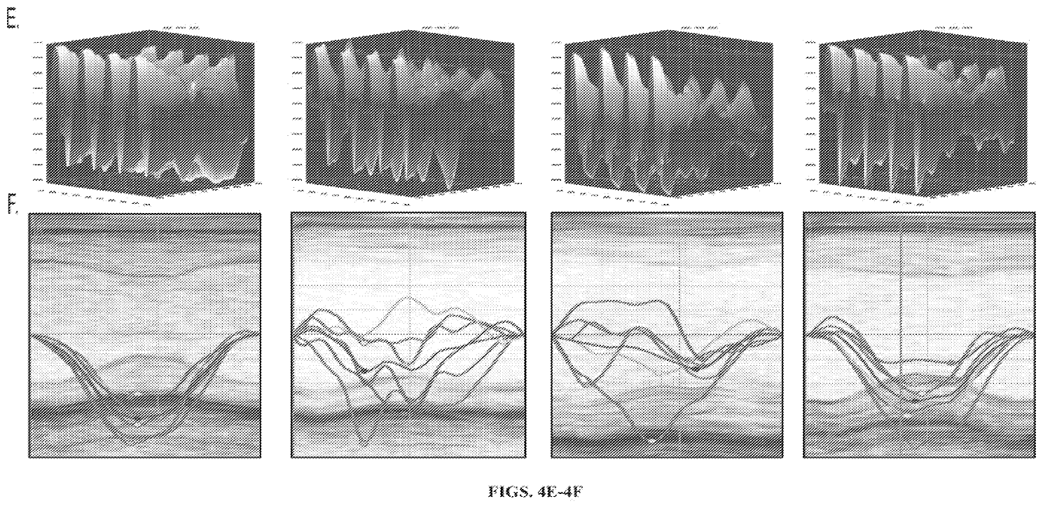

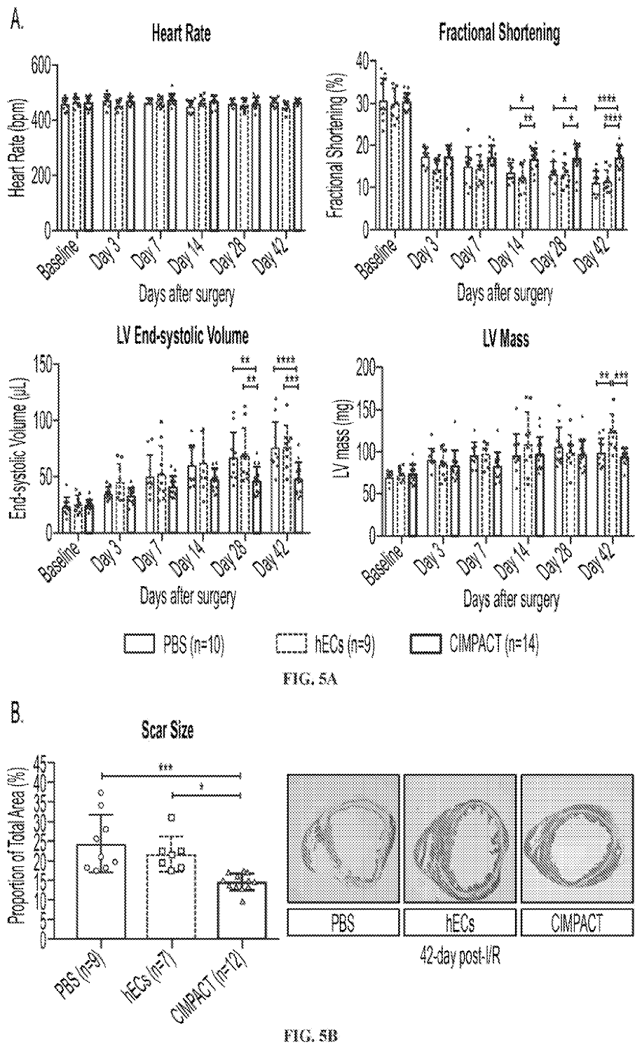

CIMPACT attenuated structural remodeling and scar formation after ischemia/reperfusion: Analysis of short-axis M mode ECHO were performed to evaluate the structural remodeling of the heart. Fractional shortening (FS) determined by M mode LV tracing showed a significant reduction after I/R, but were preserved in mice treated with CIMPACT compared to hECs and PBS. LV mass were significantly higher in hECs treated group at 42-day post-I/R. Consistently, hECs treated mice showed a trend of higher heart weight to body weight and heart weight to tibia length ratio compared with CIMPACT treated mice (FIG. 10B). Although Stroke Volume showed no difference between groups at each time points, mice treated with hECs and PBS have significantly higher LV end-diastolic and end-systolic volume compared with CIMPACT treated mice (FIG. 5A). ECHO analysis suggested CIMPACT treated mice have less chamber dilation and compensated cardiac hypertrophy. With Masson's Trichrome staining, it was determined that CIMPACT treated mice have significantly smaller chronic scar size at 42-day post-I/R compared with mice treated with hECs and PBS. Overall, these findings evidence that CIMPACT treatment preserved cardiac function and attenuate cardiac remodeling and scar formation long-term post-I/R.

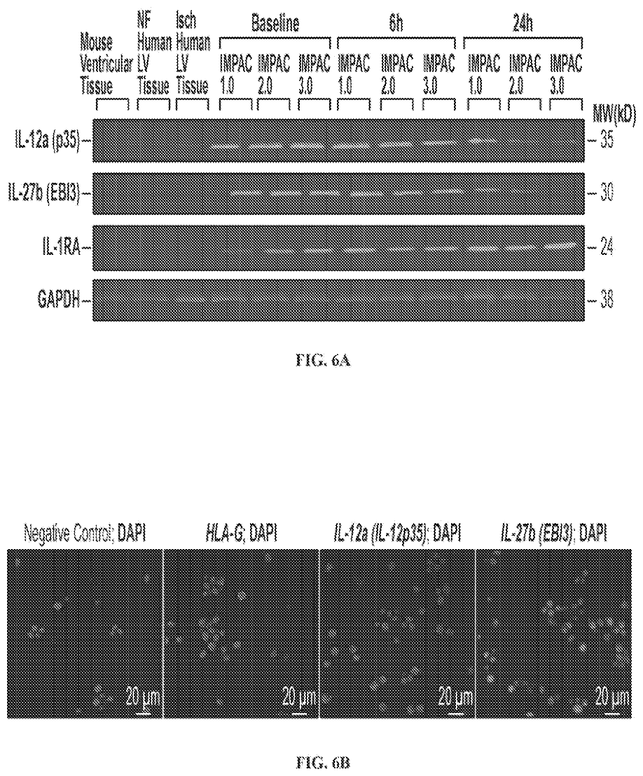

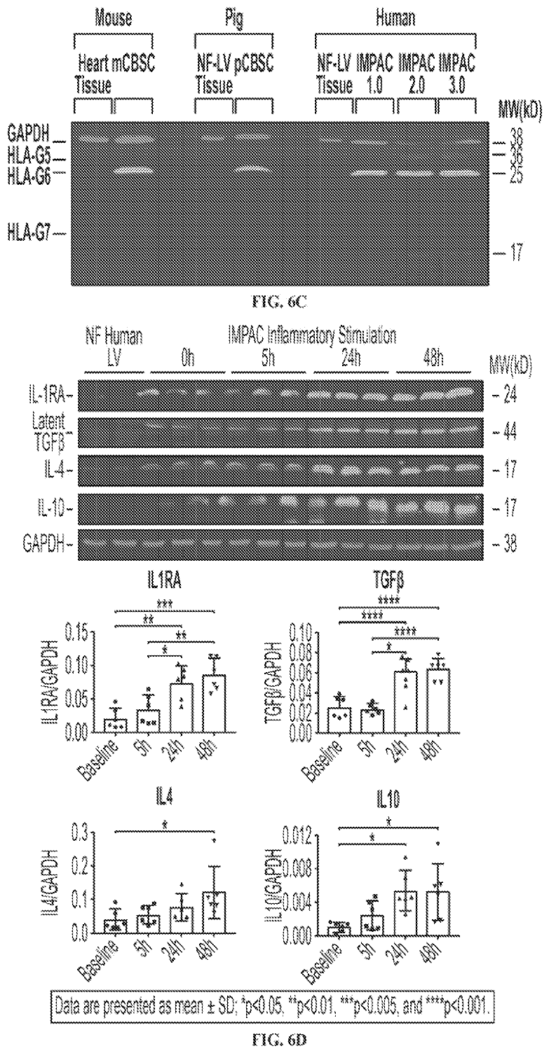

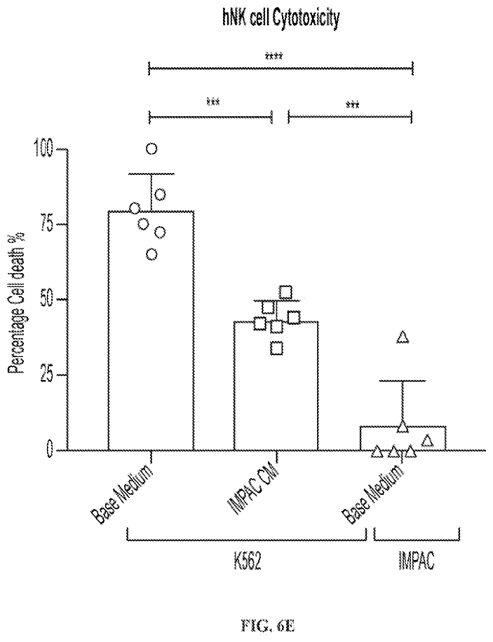

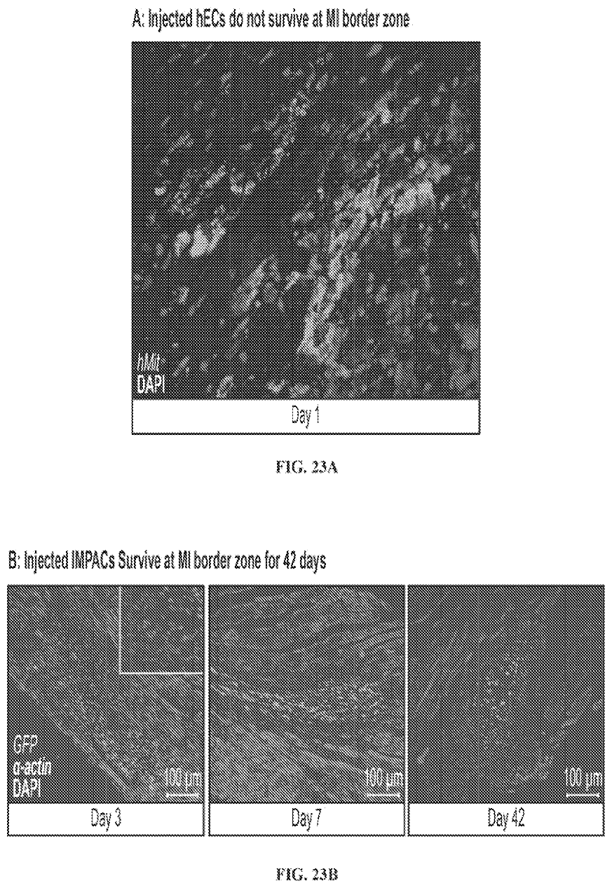

IMPACs have ability to evade immune response (FIGS. 6A-6E): The finding that IMPACs were still present in the mouse MI border zone 6 weeks after injection suggests they may evade the immune response (FIGS. 23A, 23B). IMPAC expressed all three soluble isoforms of sHLAGs with HLA-G6 being the most predominant sHLA-G isoform (FIG. 6A). The positive protein expression of sHLA-G was confirmed by immunofluorescence experiment (FIG. 6B). HLA-G is a potent immune inhibitory molecule. In addition, the IMPACs also constitutively produced the immunoregulatory factors; IL-4, IL-IRA, TGFb, IL-10, IL-12a and its binding partner IL-27b to form IL-35 (FIG. 6C and FIG. 6D). All these molecules may allow IMPACs to evade NK cell cytotoxicity. To further confirm the idea that the IMPACs have the ability to evade human NK cells (hNK cells), the NK cell cytotoxicity assay was performed using an LDH Cytotoxicity Assay Kit (LSBio). We found that IMPAC cell death was very low (<5%) with all of the treatments. The cytotoxic death of K562 cells was 87.69% and 85.62% at 1 to 5 NK cell groups with or without IL-2 stimulation respectively. IMPAC condition medium reduced these cell death rate to 43.13% and 7.81% respectively. These results provide evidence that the SCBSCs have the ability to evade NK cell cytotoxicity and the SCBSC secretome contained within the condition medium have a protective effect against the NK cell induced cytotoxicity (FIG. 6E and Table 4).

TABLE 4
hNK cell Cytotoxicity Assay

| | Percent Cell Death | | | |
|---|---|---|---|---|
| | K562 Death (%) | K562 Death (%) | IMPAC Death (%) | IMPAC Death (%) |
| | Base Medium | Condition Medium | Base Medium | Condition Medium |
| 1:5 NK cells + | 87.69 | 43.13 | 0.005 | 0.00049 |
| IL-2 Stimulation | | | | |
| 1:5 NK cells | 85.62 | 7.81 | 1.63 | 0.975 |
| 1:2.5 NK cells | 2.235 | 0 | 0.655 | 1.8505 |
| 1:1.25 NK cells | 6.34 | 2.585 | 1.66 | 0.755 |
| 1:0.625 NK cells | 10.61 | 1.105 | 3.32 | 0 |

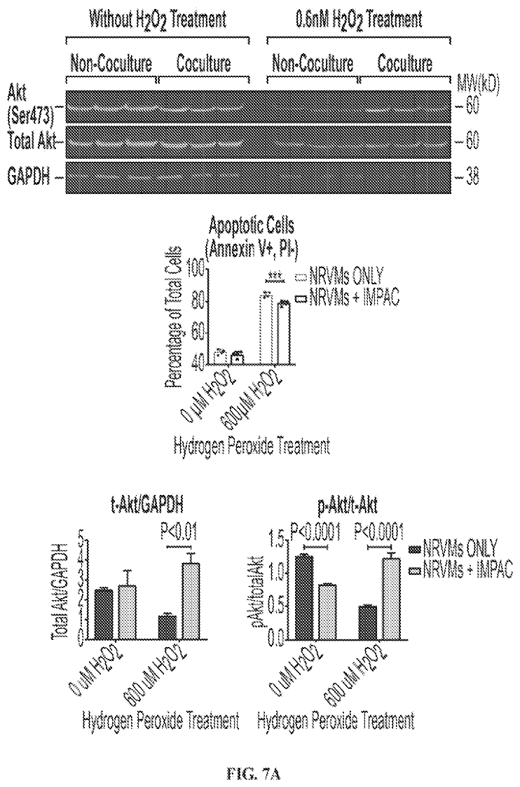

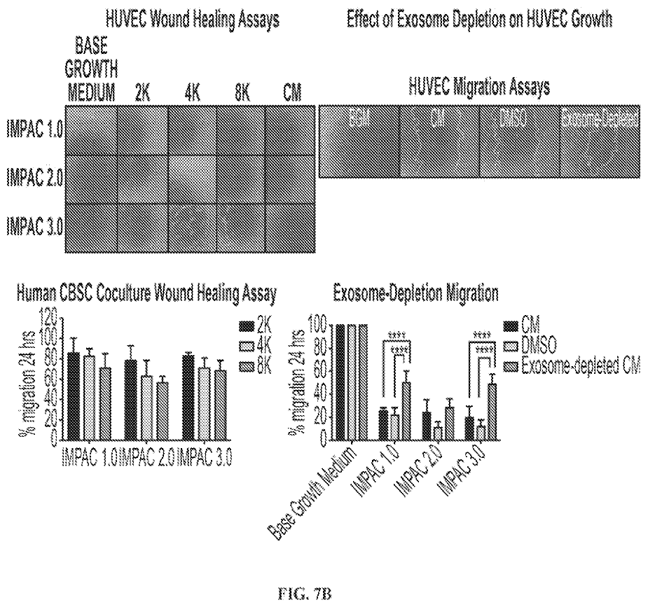

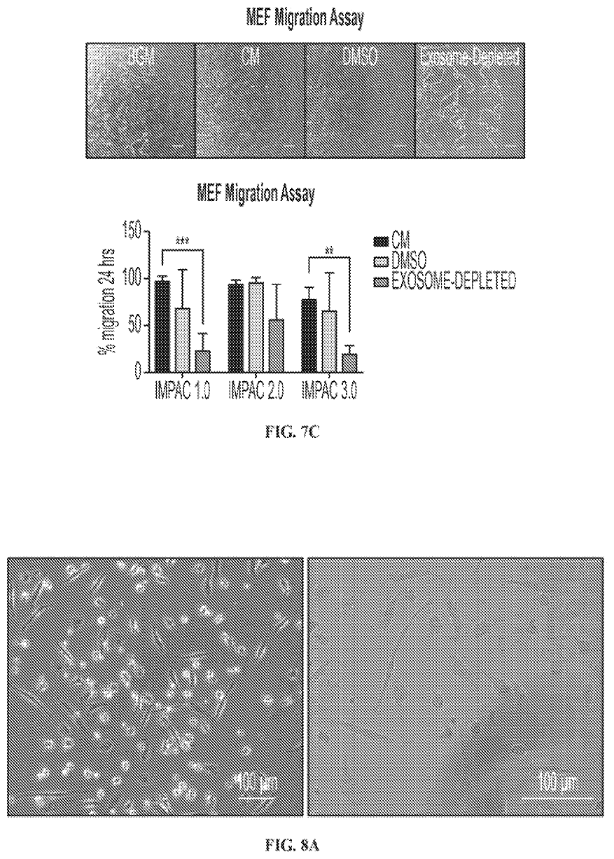

IMPAC Secretome have enhanced wound healing abilities: (FIGS. 7A, 7B and 7C): To determine if IMPACs secrete factors that rescue cardiac myocytes from a severe oxidative stress, total Akt abundances and levels of Ser473 phosphorylation were analyzed. The NRVMs co-cultured with IMPACs exhibited significantly reduced rates of apoptotic cell death, preserved total Akt abundance, and increased rate of Akt phosphorylation. Co-culture of NRVM with IMPACs under control conditions reduced the rate of Akt phosphorylation, providing evidence that the IMPACs secrete factors that bi-directionally regulate the cardiac myocyte survival and growth (FIG. 7A).

Concurrently, the effects of the IMPAC secretome were tested on human umbilical vein endothelial cells (HUVECs) using a wound healing assay. The rate of HUVECs wound closure was significantly reduced when co-cultured with IMPAC phenotypes in a dose dependent manner. The co-cultured HUVECs with all of the phenotypes exhibited similar growth inhibitory tendencies (FIG. 7B-(1)). GW4869, an inhibitor of exosome generation supplemented during the condition media collection significantly reversed the effects of condition media induced HUVEC growth suppression (FIG. 7B-(2)). The same IMPAC conditioned media significantly increased mouse embryonic fibroblast (MEF) growth and GW4869 inhibited the growth (FIG. 7C). These results indicate that the IMPAC exosomes secreted into the condition medium have a role in regulating the behavior of endothelial and fibroblast cells necessary for wound healing.

IMPAC Exosomes harbor miRs with multiple possible effects (Tables 5A & 5B): To broaden the understanding of the mechanism of IMPAC exosome-mediated cell regulation, micro RNAs contained in the IMPAC exosomes were analyzed utilizing Exo-NGS exosomal RNA sequencing service (System Biosciences). As a first step of analyses, 30 micro RNAs (miRs) were selected that had the most sequence reads. Also included were their micro RNA families that are expected to have similar functional effects to the list (Table 5A). Their effects were then examined. miRs were found that could explain post MIR CIMPACT treatment such as cardioprotection, reductions in inflammation, fibrosis, and hypertrophy, and promotion in angiogenesis and cellular growth. miRs that have known effects that might could lead to tissue injury were also found (Table 5B).

Using online miRDB (7, 8), more specific targets of the miRs were further analyzed in three functional areas; immunoregulation, cell survival/cell growth pathways, and cell metabolism. Generally, IMPAC exosomes had a greater quantity (number of reads) of miRs compared to hBMSCs analyzed in all three functional areas. In the immunoregulation area, miRs were found that inhibit pro-inflammatory cytokines, IL-6, TNF, IL-1β, IL-1α, and their downstream transcription factors, STAT1, STAT2, and STAT3. miRs that target IL-2, IL-7, IL-15, and their downstream transcription factor, STAT5; IL-1β and its downstream factor STAT6, were also found. miRs that target INFγ were not found but miRs that target its downstream transcription factor, STAT4, were. The miRNAs that target anti-inflammatory cytokines TGFβ2 and TGFβ3 were also found. The toll-like receptor (TLR) pathway is responsible for activation of innate immunity. Its activation leads to NF-kB nuclear translocation and activation of inflammatory cytokine gene program (9). The IMPAC exosome did not contain miRs that target TLRs directly; however, they contained miRs that target important molecules for the TLR signaling pathway; IRAK1, IRAK3, TRAF6, PDCD4, and NF-kB1. Further, the exosomes contained miRs that target Importin 3a that is responsible for transporting NF-kB to the nucleus. Finally, IMPAC exosomes contained miRs that target the pro-inflammatory and co-stimulatory molecule, CD40LG (Tables 5C and 5D). These results demonstrate that IMPAC exosome harbor miRs with the potential to influence the immune response to injury.

IMPAC Secretome has Immunomodulatory Effects: Injection of CIMPACT into the border zone of an MI in mice reduced the acute increase in neutrophil infiltration into the injured heart, reduced infarct size and improved myocardial function. Previous studies have shown that that enhanced recruitment of immune cells and prolonging inflammation are major causes of infarct enlargement and subsequent fibrosis. Without wishing to be bound by theory, the hypothesis here is that IMPACs secrete paracrine factor, including exosomes, that modulate post MI inflammatory processes to improve wound healing and promoting beneficial structural and functional remodeling. To test this hypothesis in vitro, TNFα, IL-1β, and INFγ were added to mimic an inflammatory environment and tested to see if paracrine factors from IMPACs suppresses production of factors that promote recruitment of immune cells and inflammation.

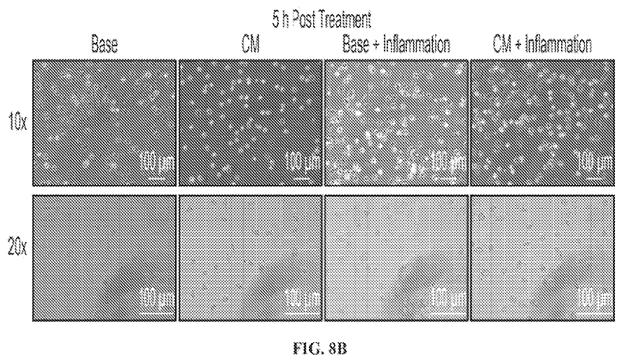

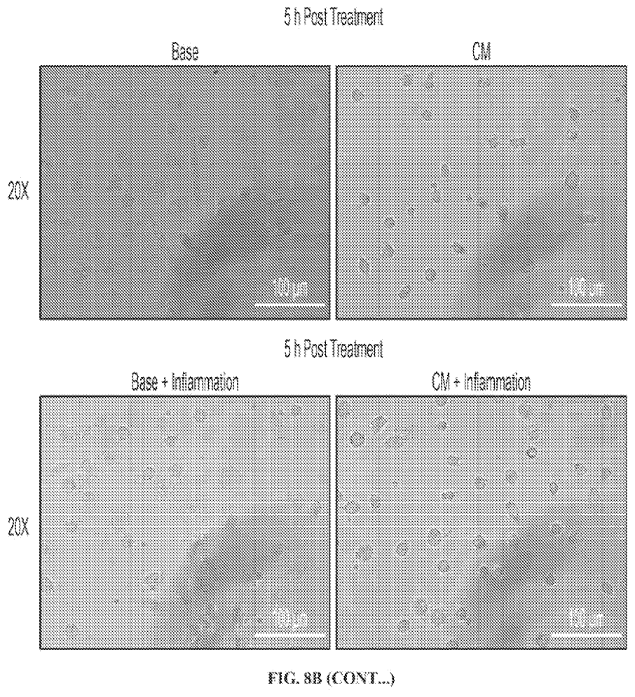

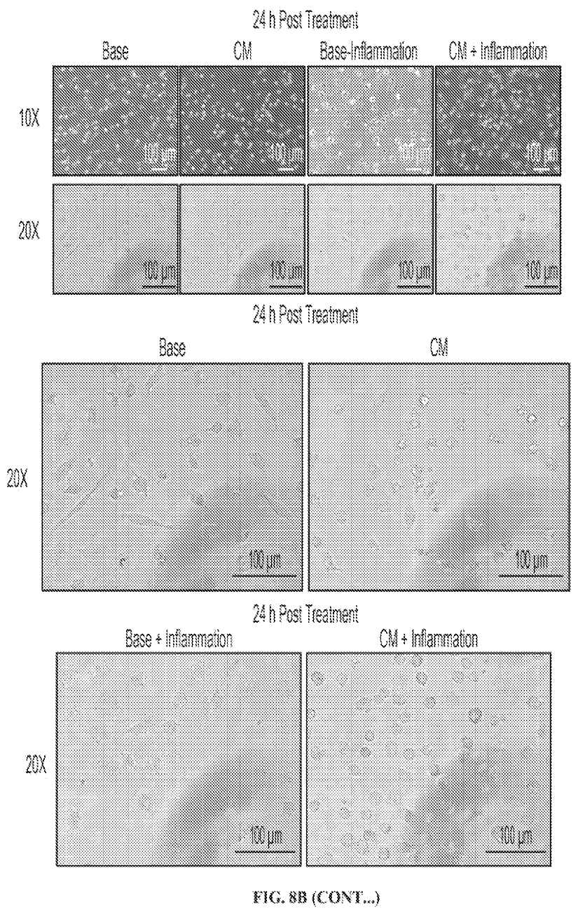

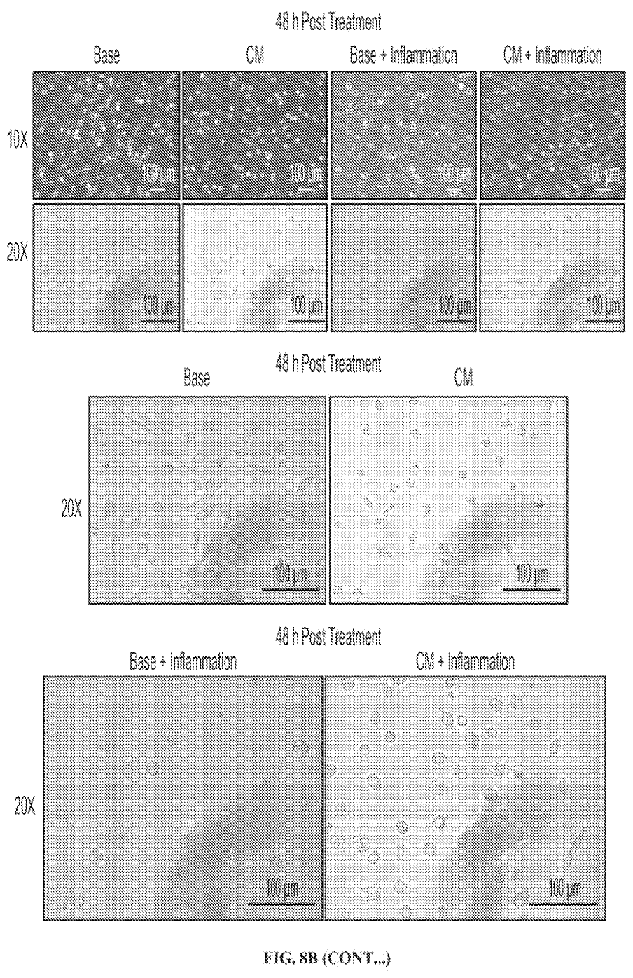

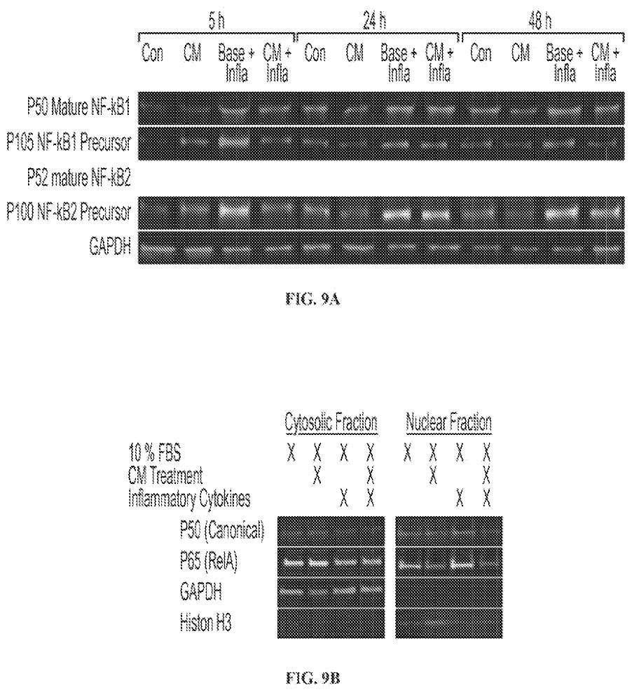

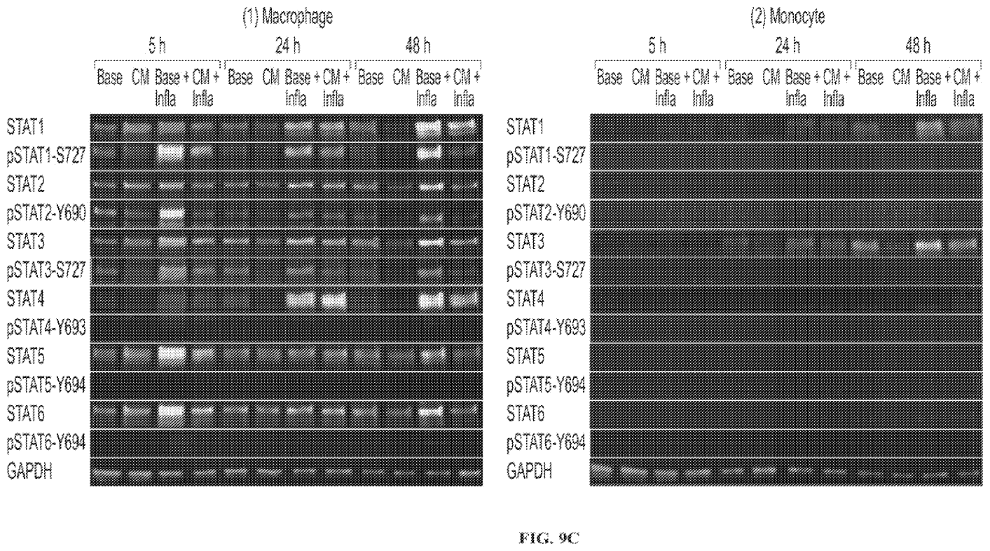

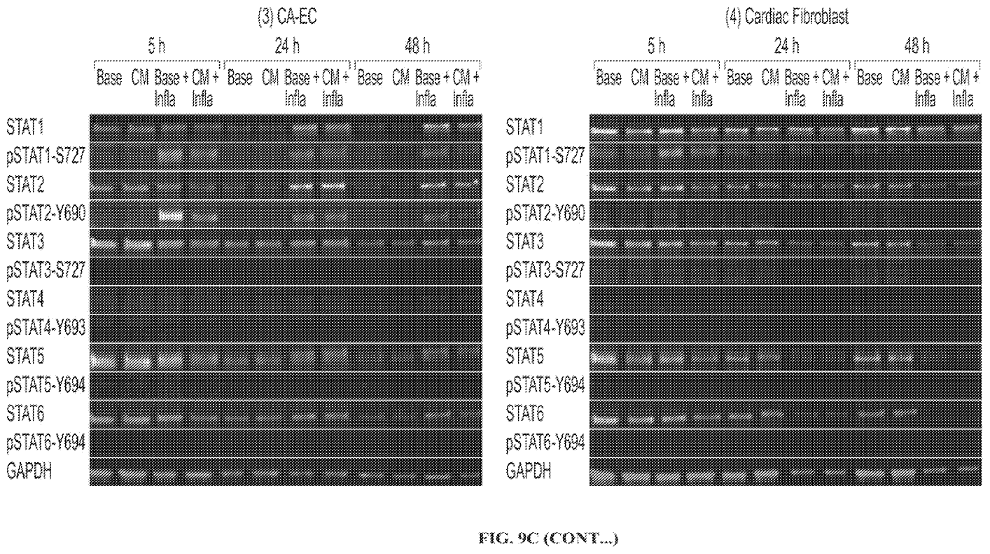

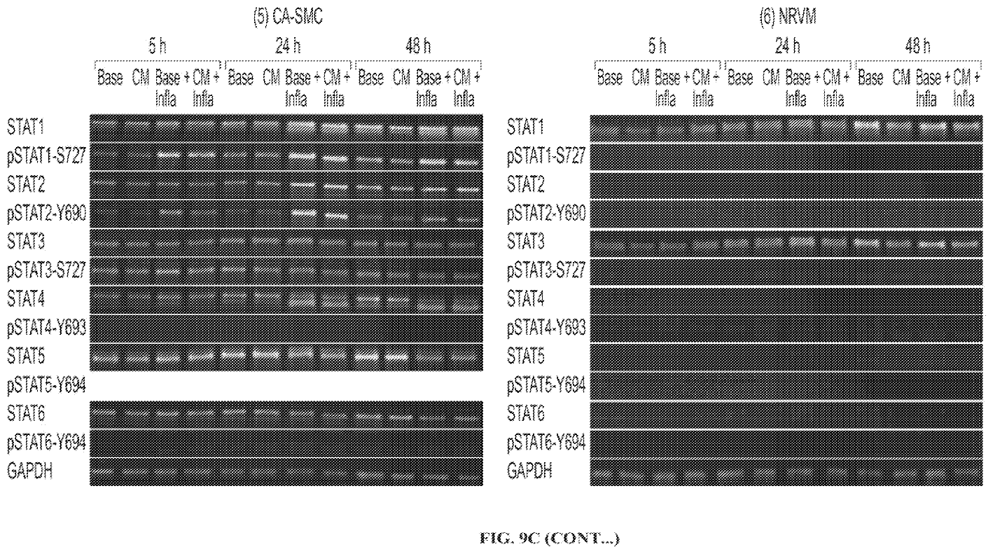

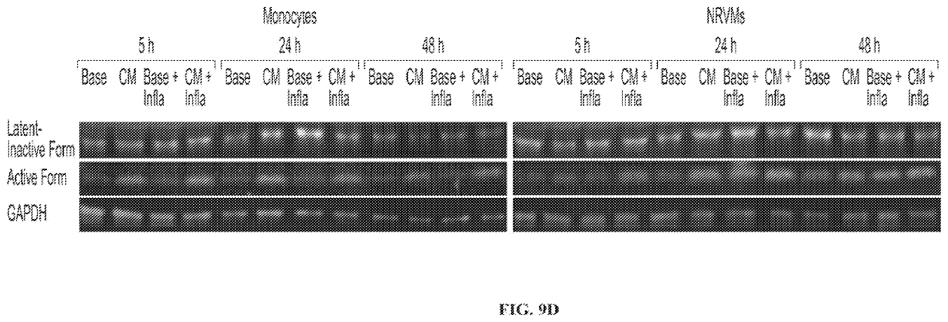

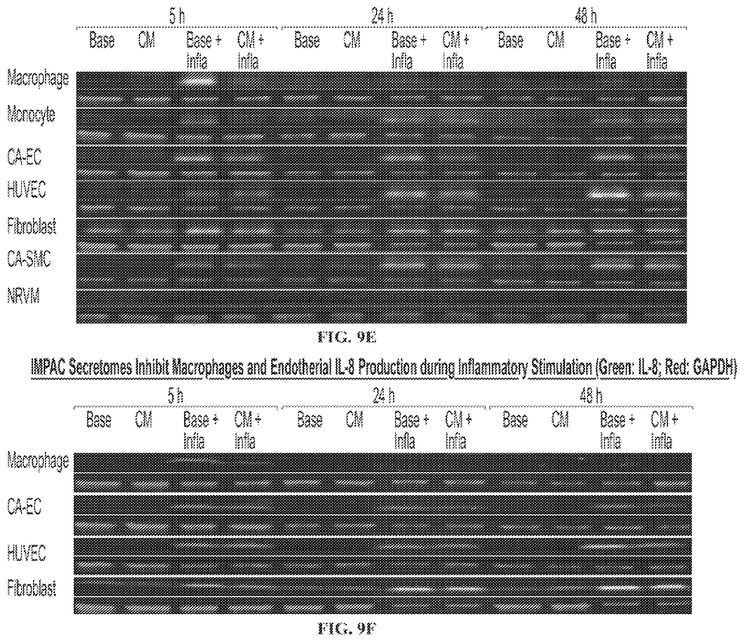

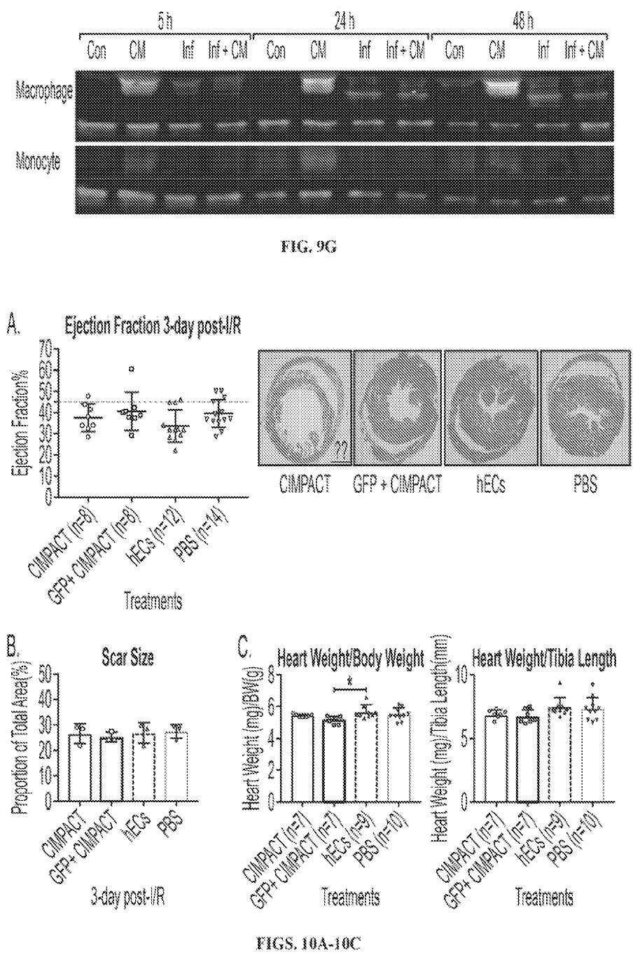

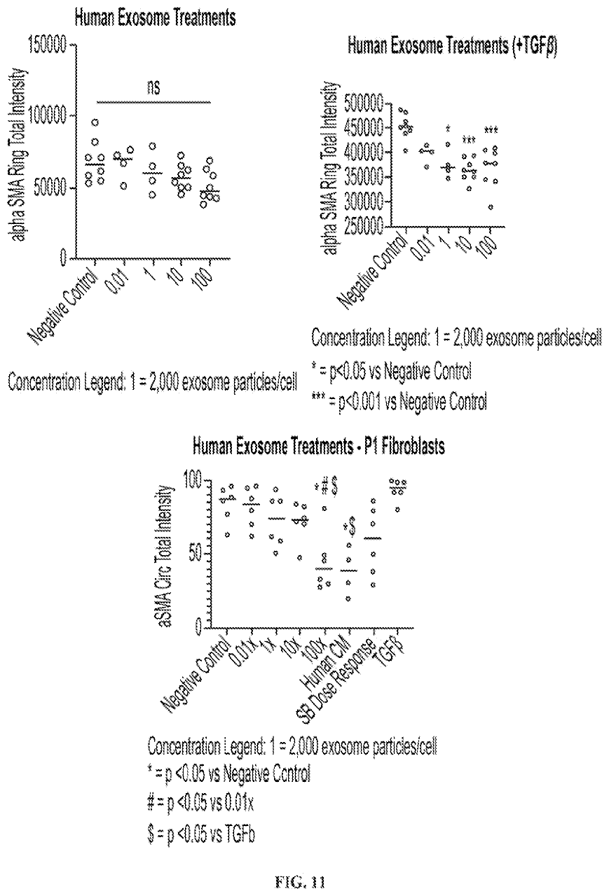

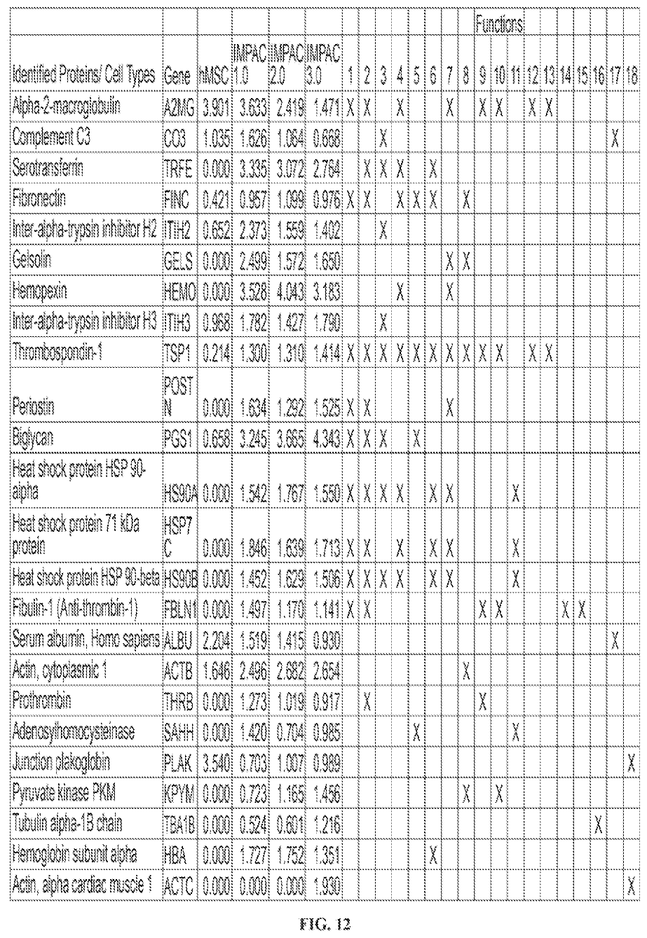

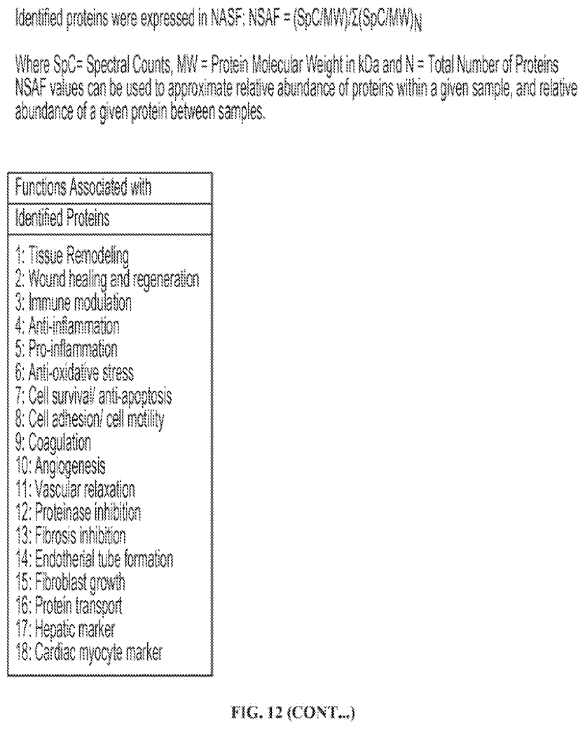

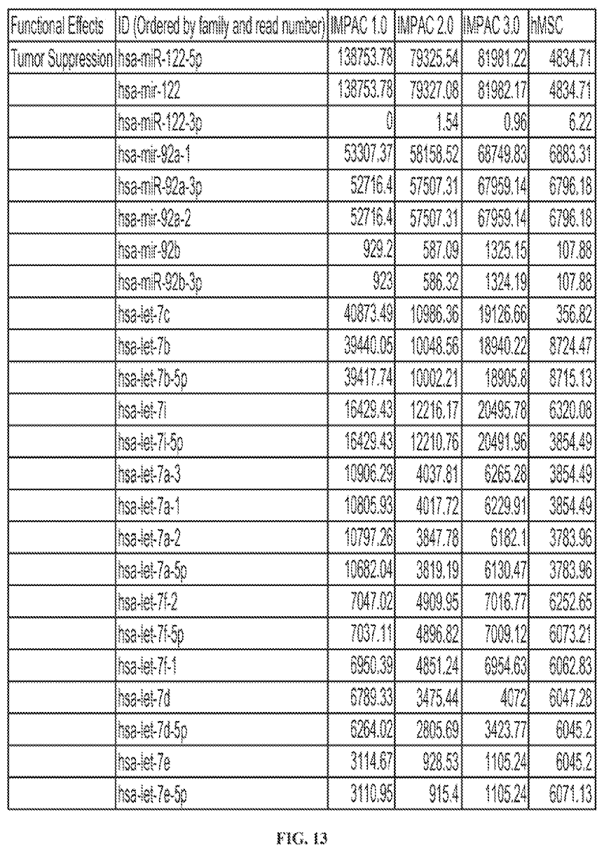

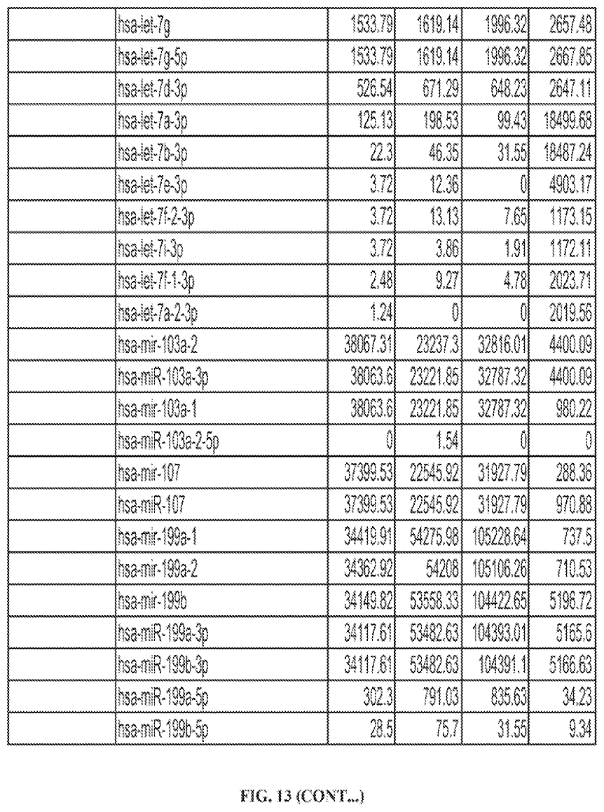

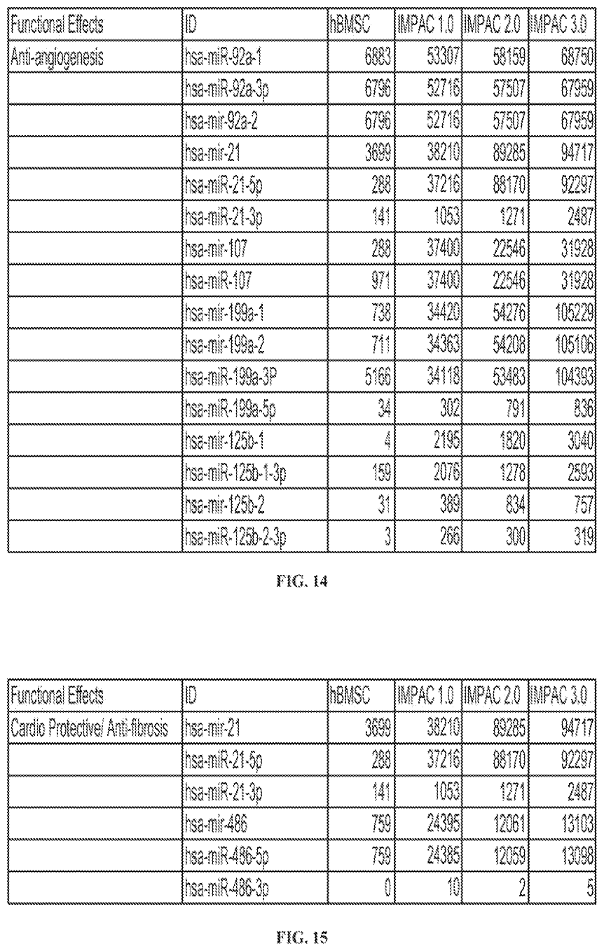

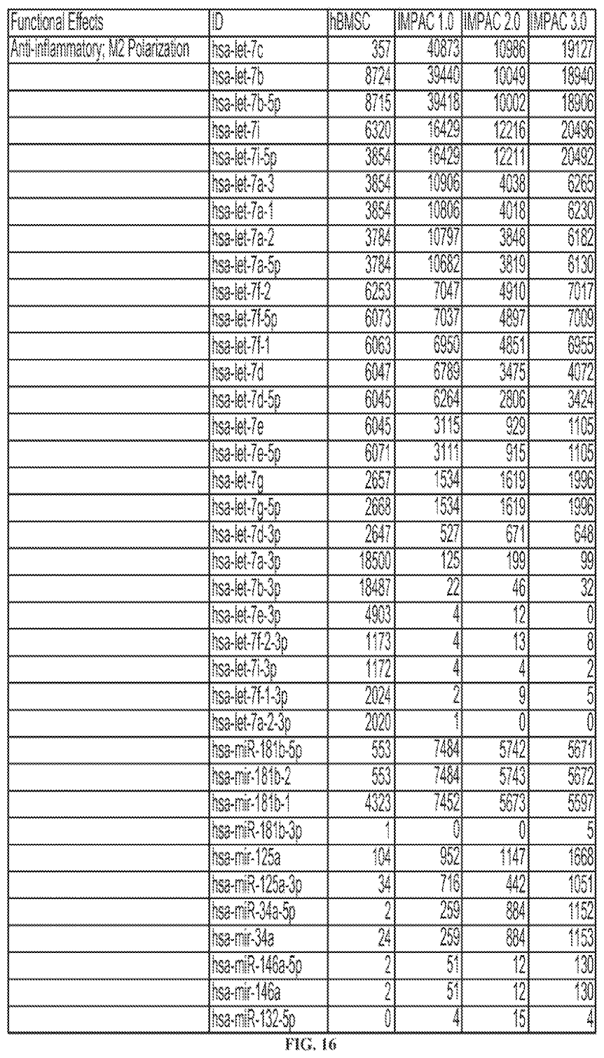

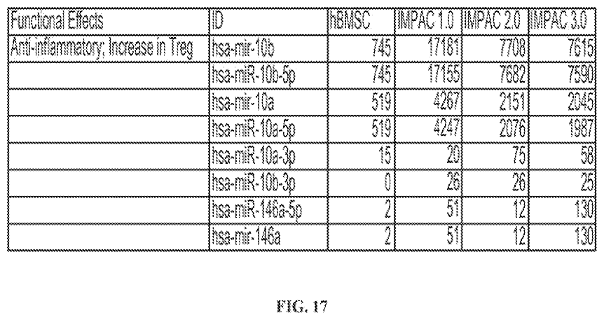

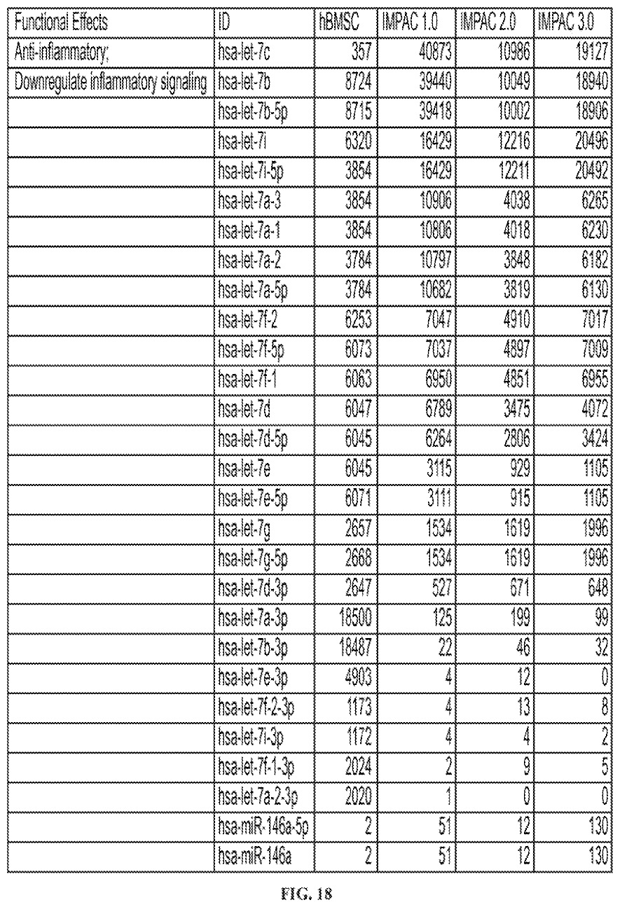

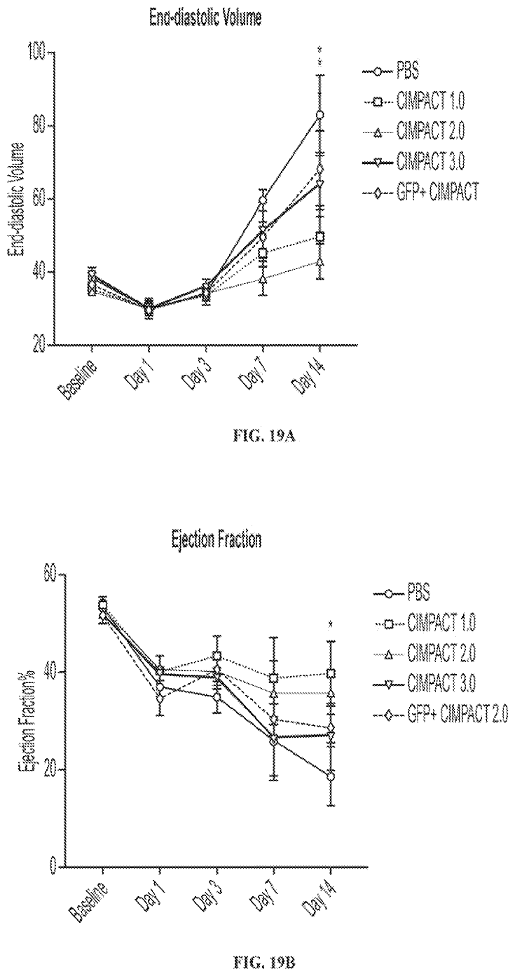

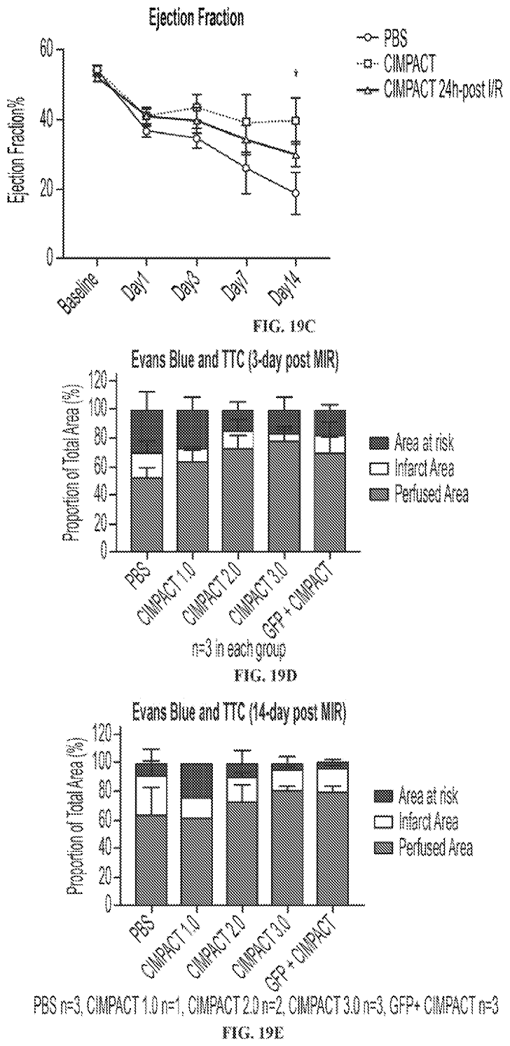

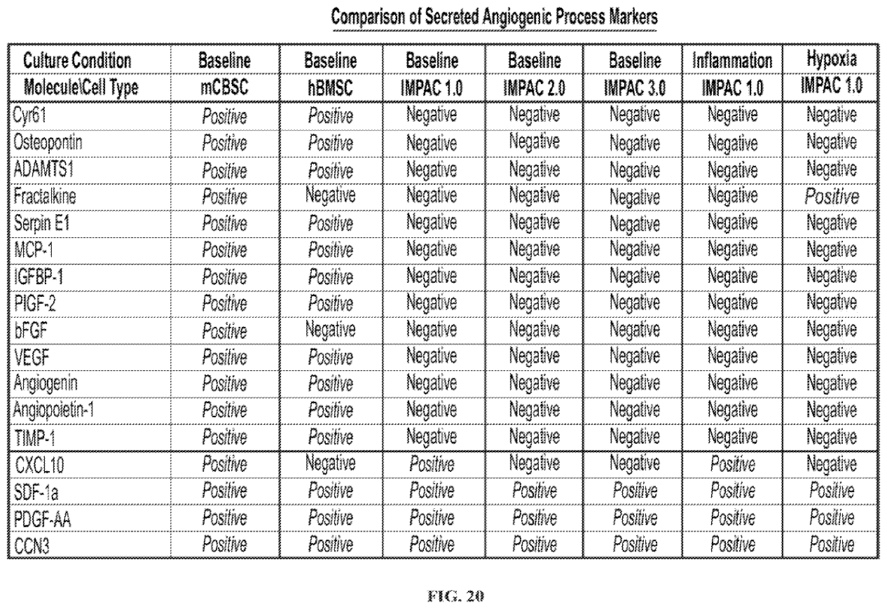

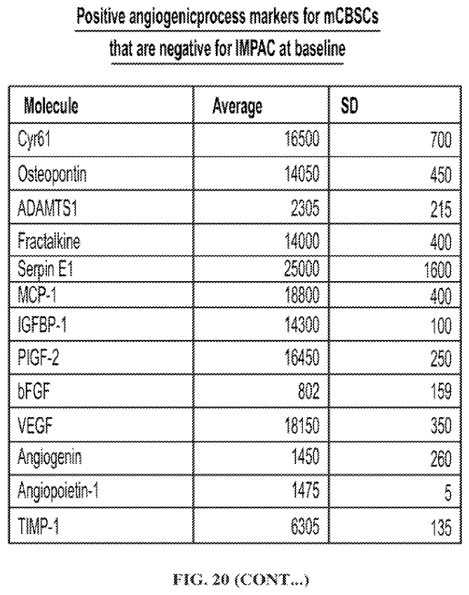

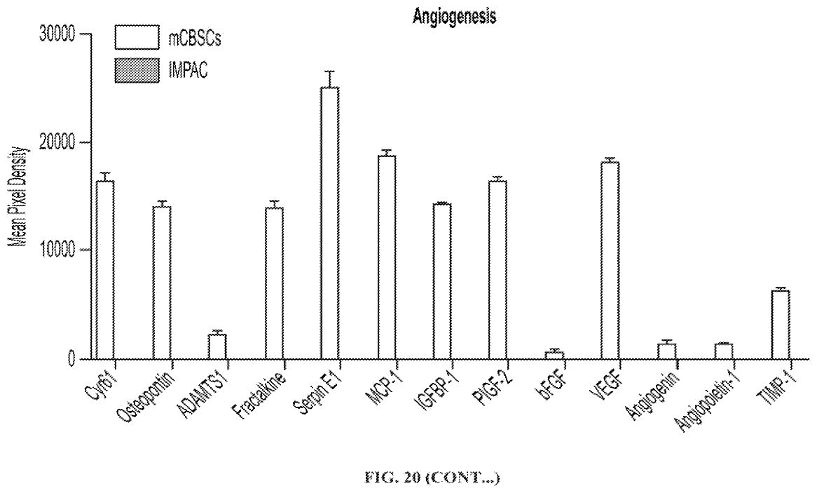

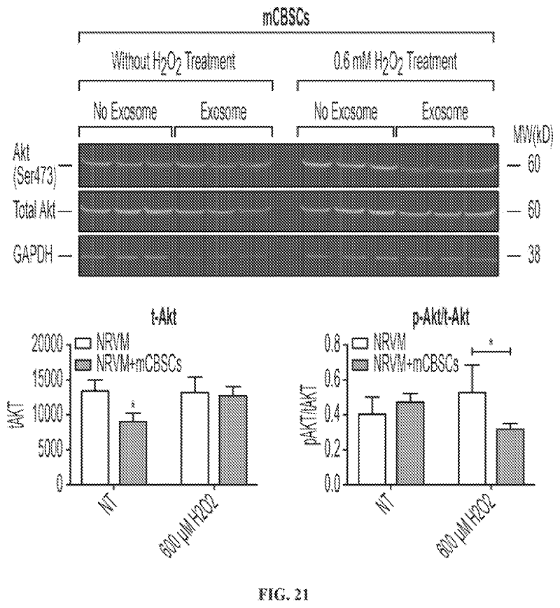

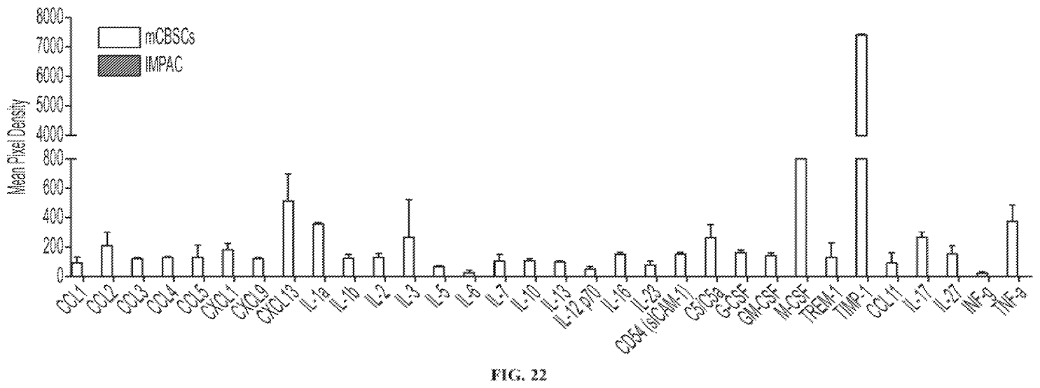

IMPACs-CM cause changes in Macrophage Morphology to Resemble Unpolarized Monocytes and reduce macrophage NF-kB activation (FIGS. 7A-7C, 8A and 8B). Monocytes from human peripheral blood mononuclear cells (hPBMNCs) were differentiated into macrophages with a macrophage differentiation medium containing M-CSF. hPBMNCs derived macrophages (hPBDMs) were exposed to IMPAC condition medium (CM) at under basal conditions and after exposure to the inflammatory cocktail for 5, 24, and 48 h. The differentiation protocol induced a mixture of three macrophage phenotypes; 1) monocyte-like small, loosely attached cells, 2) M1 (inflammatory) macrophage-like cells that were flat, large, and had a fried egg appearance, and 3) elongated M2 (regulatory) macrophage-like cells. The Base medium contained 10% FBS shifted the initial phenotype more toward the M1, pro-inflammatory phenotype. The inflammatory cocktail greatly augmented that shift and the hPBDMs became large, flat, and vacuolized cells (FIG. 7A). The CM treated hPBDMs caused a shift toward the monocyte like phenotype. The inflammatory treatment of hPBDMs did not transform them to the flat and vacuolized M1 polarized cells. These morphological data provide evidence that IMPAC-CM has ability to inactivate hPBDM polarization. TGFβ and IL-10 within the IMPACs secretome may have produced these effects. NF-κB has been recognized as a central mediator of the human immune response because of its ability to respond to a large variety of activating factors and then regulate the expression of inflammatory cytokines, chemokines, immunoreceptors, and cell adhesion molecules (10). Therefore, it was tested whether IMPACs-CM alter the hPBDM NF-κB activation status. It was found that IMPACs-CM increased both precursors at 5 h and then decreased these precursors at 24 and 48 h compared to the respective baseline control. During the inflammatory stimulation, IMPACs-CM consistently reduced these precursor abundances (FIG. 9A). The IMPACs-CM treatment induced reduction in p50 NF-κBd1 and p65 RelA nuclear translocation compared to the baseline control. These reductions were also seen during inflammatory stimulations in THP-1 cell derived macrophages (THPDMs). The IMPACs-CM also reduced NF-κB activity as confirmed by reduced NF-κB nuclear translocation; however, this reduction may be an effect of IMPACs-CM induced reduction in total NFkB precursor availability (FIG. 9B). NF-κB knockout and blockade have been shown to improve post MI survival, reduce infarct size, reduce left ventricular dilatation, and preserve cardiac functions (11-14). These results provide evidence that the IMPACs-CM exerts anti-inflammatory effects suppressing NF-κB abundance and activity contributing to improved post-MI remodeling.

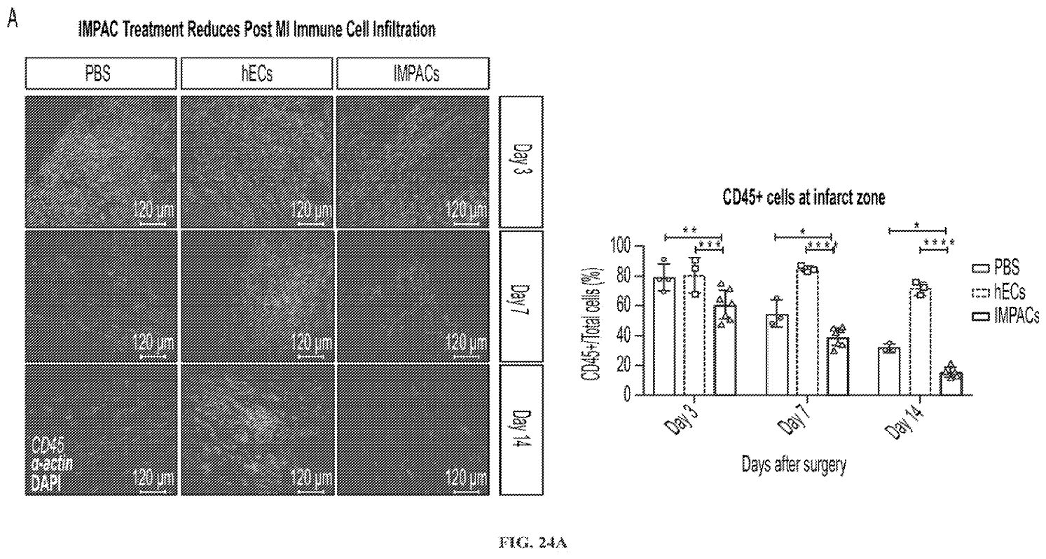

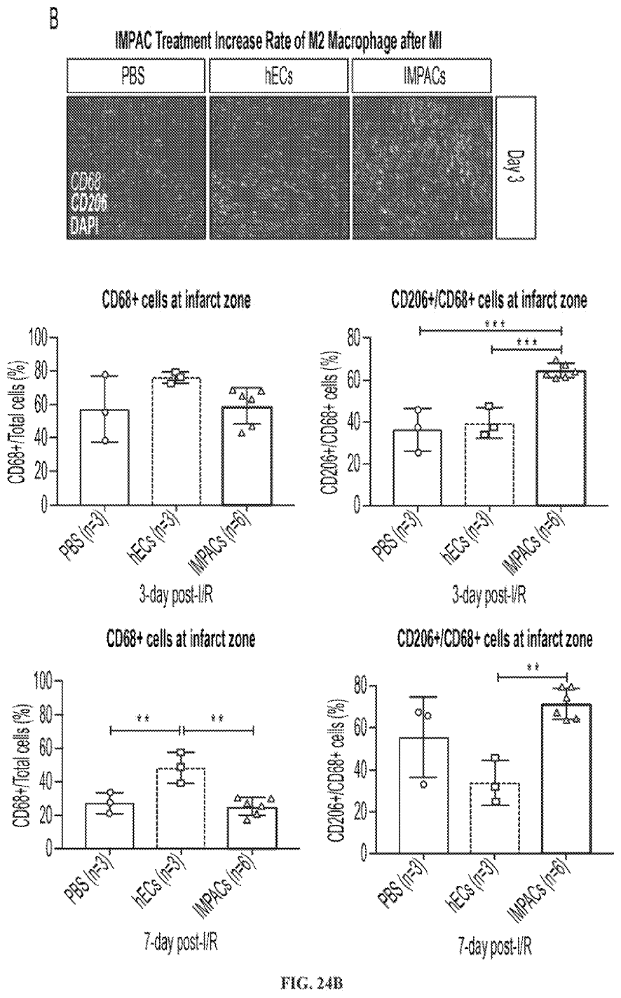

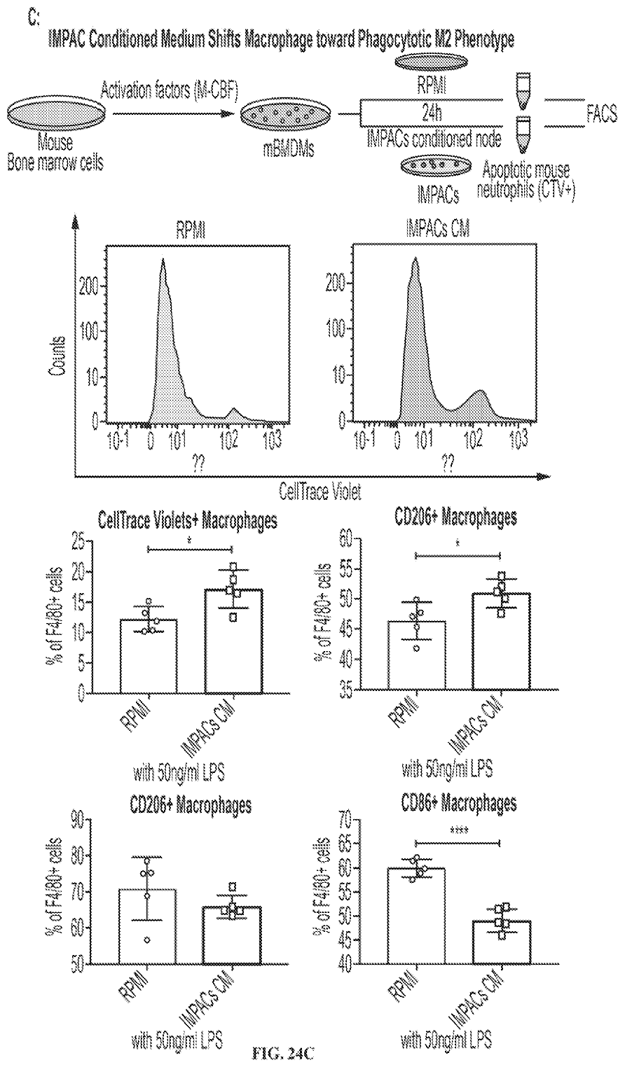

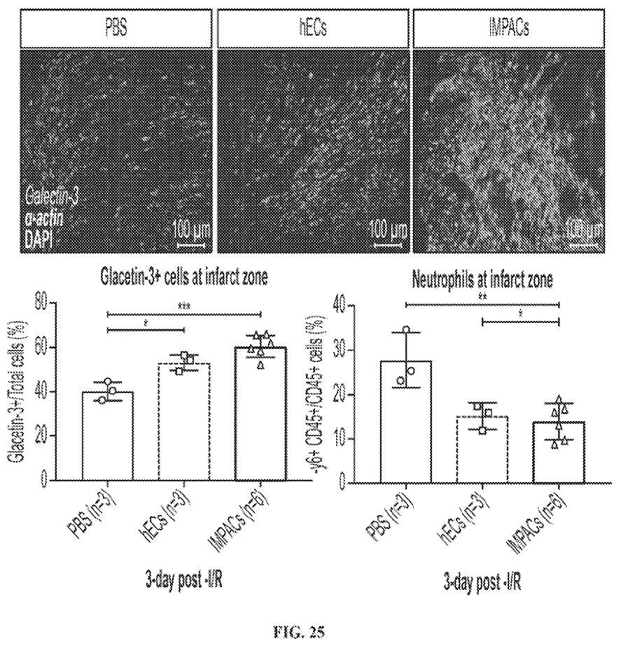

IMPAC treatment reduces post MI immune cell infiltration and shifts macrophages to a M2 phenotype: Using CD45 as a marker for total immune cells, the number of CD45 positive (CD45+) cells were determined as a percent of the total nuclei to determine total immune cell infiltration into the infarcted heart. IMPAC treated mice had significantly lower percent of CD45+ cells versus hearts treated with PBS or hEC. hEC were used as a human control cell to be compared to IMPAC treatment. hEC engraftment was observed at 1-day post I/R, but not at the 3-day post-I/R (FIGS. 23A, 23B). The hEC treated mice had the greatest CD45+ cell infiltration suggesting that the xenogeneic rejection of the human cells augmented the post IR inflammatory response (FIG. 24A). To determine if IMPAC treatment contributes to a shift in immune cell phenotype we measured CD206, a marker for M2 macrophages and CD68 a pan-macrophage marker. Consistent with the CD45 data, the hEC treated myocardium had the greatest number of macrophages in the infarct zone. IMPAC treated myocardium had the greatest CD206 to CD68 ratio indicating that IMPAC treatment induces M2 macrophage polarization (FIG. 24B). Galectin-3 staining was performed to assess phagocytotic macrophage activity. These studies showed that IMPAC treated myocardium contains the greatest % of galectin-3+ cells (FIG. 25)

The ideas were further explored with in vitro experiments. To determine if the IMPAC secretome promotes phagocytosis, mouse bone marrow cells were differentiated with M-CSF to mouse bone marrow derived macrophages (BMDMs). BMDMs were then exposed to pre-conditioned with either control media (RPMI) or IMPAC conditioned medium (CM) for 24 hours and co-cultured with CellTrace Violets labeled apoptotic mouse neutrophils for 60 minutes. The rate of phagocytosis was evaluated with counting CellTrace Violet+ BMDMs using flow cytometry. These studies show significant increases in CellTrace Violet+ BMDMs with IMPAC-CM treatment, indicating that the IMPAC-CM promotes BMDM phagocytosis. Additionally, IMPAC-CM significantly lowered the rate of CD86+ BMDMs and increase the rate of CD206+ BMDMs in LPS activated BMDMs further evidencing that the IMPAC-CM induces an M2 macrophage phenotype. (FIG. 24C).

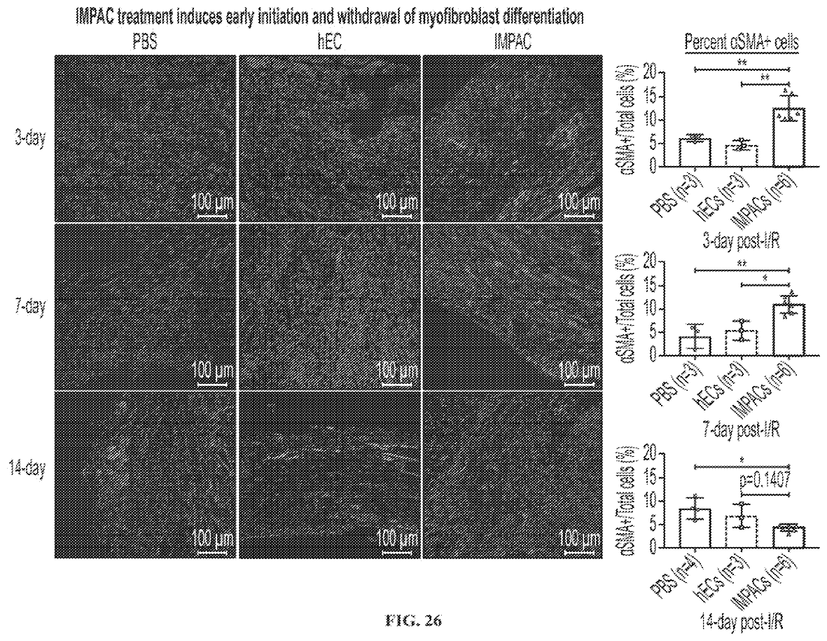

IMPAC treatment induces early initiation and withdrawal of myofibroblast differentiation: Fibroblast phenotypes are altered after IR injury, to remodel the extracellular matrix and form a scar. aSMA+ myofibroblast are a signature of fibroblast activation. A prominent aSMA+ myofibroblast differentiation was only detected within the infarct zone of IMPAC treated myocardium three days after the induction of MI injury. Fourteen-days later, PBS and hEC treated myocardium contained a significantly greater number of aSMA+ myofibroblast. The early myofibroblast activation and subsequent withdrawal found in the IMPAC treated myocardium suggests that the IMPAC treated myocardium transitioned more rapidly into reparative phase of post IR wound healing (FIG. 26). The myocardium treated with PBS and hEC, on the other hand, exhibited longer inflammatory period, late initiation of the reparative phase, and the aSMA+ myofibroblasts stay active for prolonged period of time. These responses may lead to less extensive myocardial damage and a smaller fibrotic scar in IMPAC-treated hearts (FIG. 26).

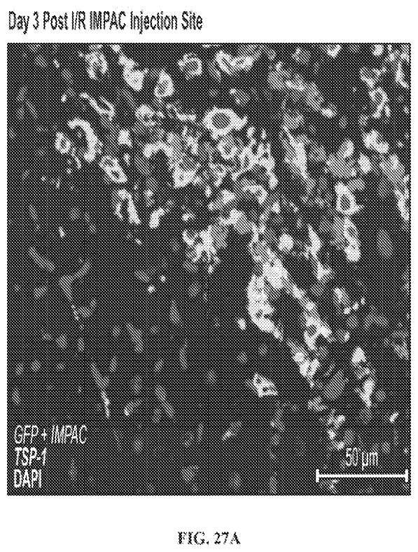

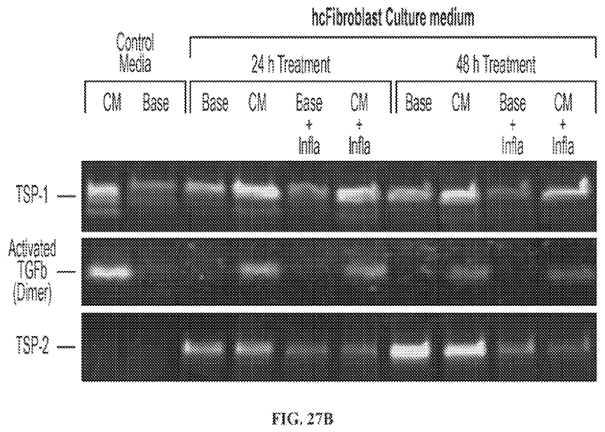

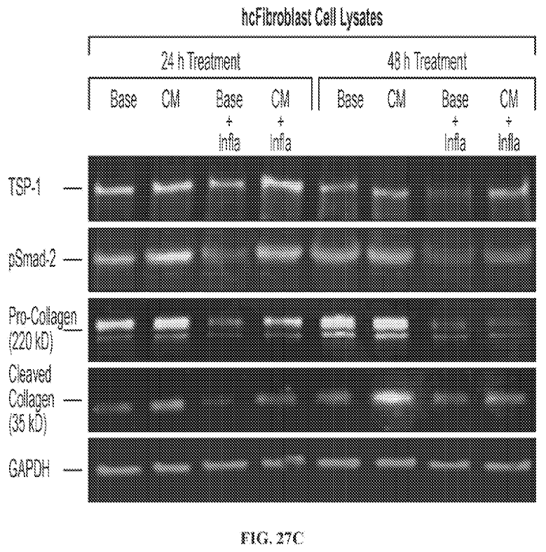

IMPACs secretome includes Thrombospondin-1 (TSP-1) and activated TGFβ: Western analysis was used to define secreted factors that might explain the improved post MI wound healing in IMPAC-treated hearts. These analyses revealed that IMPAC conditioned medium (CM) contains TSP-1 (FIG. 27B). Another thrombospondin family protein, thrombospondin-2 (TSP-2), implicated to delay wound healing, was only detected in the hcFibroblast culture (FIG. 27B). TSP-1 is an adhesive matrix glycoprotein. TSP-1 adhered to matrix protein can be detected after the secretion. Using immunofluorescence staining TSP-1 secreted by IMPACs and bound to the extracellular matrix was detected at the MI border zone IMPAC injection sites (FIG. 27A). TSP-1 binds to latency-associated peptide of immature TGFβ complex and enzymatically releases mature biologically active TGFβ. The active forms of TGF β (Dimer: 25 kD and monomer: 12.5 kD) were detected only in the CM. (FIG. 27B).

IMPAC secretomes support maintenance of collagen-I synthesis during inflammatory stimulation: Human cardiac fibroblast cells (hcFibroblasts) were cultured with or without IMPAC-CM under influence of inflammatory cytokines (TNFα, INFγ, and IL-1β). Effects of IMPAC-CM on TSP-1/TGF β signaling pathway factors and the downstream effectors was examined using Western analysis. Compared to the control base medium, hcFibroblast culture medium had slightly elevated levels of TSP-1, indicating hcFibroblasts secrete small amount of TSP-1. Inflammatory factor treatment lowered the TSP-1 level to the baseline control. The active form of TGFβ was only detected in the original IMPAC conditioned medium and the conditioned medium treated hcFibroblast culture media (FIG. 27B). The cell lysates were also studied using Western analysis for TSP-1 and TGFβ and their downstream molecules, phosphorylated Smad-2 (pSmad-2), and collagen-I. Inflammatory treatment reduced TSP-1, pSmad-2, and collagen I protein abundance in hcFibroblast cells indicating that the inflammatory treatment suppresses the TSP-1/TGFβ/Smad-2 signaling pathway and reduces collagen-I production. The IMPAC-CM treated hcFibroblast cells maintained the TSP-1 axis protein abundances and prevented the reduction in collagen-I production for 24 hrs; however, these IMPAC-CM induced effects were attenuated at 48 hr (FIG. 27C). This attenuation may be caused by depletion of active TGFβ in the media by hcFibroblast cells (FIG. 27B). These in-vitro results indicate that IMPAC secretomes can convert hcFibroblasts toward a collagen-I producing reparative phenotype.

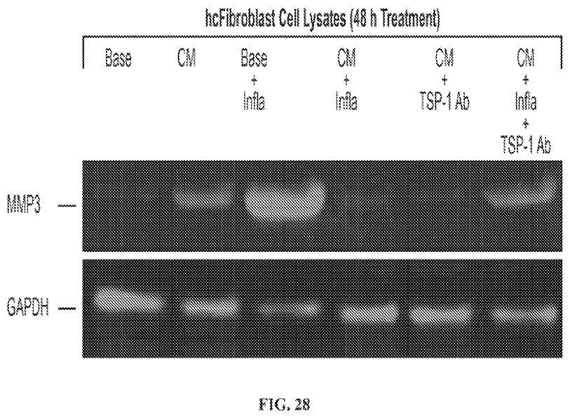

TSP-1 secreted from IMPAC inhibits inflammatory stimulation induced MMP3 production (FIG. 28): Matrix metalloproteinase (MMP) family proteins increase after MI and these effects are thought to be involved in scar formation after tissue injury. This increase correlates with LV dysfunction in heart failure patients. TSP-1 is thought to exert ECM stabilizing actions by inhibiting MMPs. Inflammatory treatment of hcFibroblasts increased matrix metalloproteinase-3 (MMP-3) production in cells. This increase was blunted by IMPAC-CM. A TSP-1 blocking antibody in the conditioned medium reversed the suppression effect, suggesting that TSP-1 secreted by IMPACs inhibits the inflammation induced hcFibroblast MMP3 production (FIG. 28). TSP-1 also activated TGFβ that has been implicated in the synthesis of protease inhibitors such as PAI-1 and TIMPs [ ]. These data suggest that the TSP-1 and TGFβ secreted from IMPACs protects the heart from post MI inflammation induced extracellular matrix de-stabilization.

Effect of IMPACs paracrine factors on STAT abundance and Activation (FIG. 9C): Many cytokines important for immune responses utilize the STAT signaling pathway, therefore the next step was to test whether IMPACs can affect STATS abundance and/or their activation. The regulation of various sets of IL-6 responsive genes, including acute phase proteins, is accounted for by the STAT3 genes (15). Similarly, STAT1 is involved in upregulating interferon-stimulated genes (ISGs) due to a signal by type I, II, or III interferons and contributes in expression of pro-inflammatory genes. STAT2 is not directly activated by IL-6; though, it was included in the analysis because STAT2 can form a heterodimer with STAT1 in response to either INFα and INFβ stimulation and contribute to the upregulation of the ISGs. STAT3 has critical functions in the immune system, including control of cytokine mediated dendritic cell production, inhibition of macrophage inflammatory signaling, and regulation of steady state and emergency granulopoiesis (16-19). In hPBDMs exposed to an inflammatory environment, IMPACs-CM increased STAT1, STAT2, and STAT3 abundance at 5 h incubation and then the abundance fell with longer incubation durations (24 and 48 h). Activation levels of these transcription factors were reduced by IMPACs-CM treatment. The inflammatory treatment greatly increased all of these STATs activation and protein abundance at all of the time points. The IMPACs-CM treatment reversed these responses. In monocytes, STAT2 was not detected at any of the time points. STAT1 and STAT3 were detected at 24 and 48 h time point but compared to the hPBDMs, those signals were much weaker. Still, the baseline reduction in STATs abundance was observed with IMPACs-CM treatment. As in macrophages, inflammatory treatment increased STAT1 and STAT3; however, activation of these transcription factors was not detected. In CA-ECs and fibroblast cells, the abundance of these STATs did not increase with the inflammatory treatment at 5 h incubation. Longer incubation with the inflammatory treatment induced slight increase in the STATs. The IMPACs-CM reduced STAT1 and STAT3 abundance and activation of STAT1 and STAT2; though, these reductions were not as prominent as the reductions observed in macrophages. NRVMs expressed relatively small amount of STAT1 and 3 initially and this amount gradually increased during 48 h of treatment with inflammatory stimuli. The inflammatory treatment did not alter the STAT1 and STAT3 abundance. Treatment with IMPACs-CM had suppressive effect in STAT1 and STAT3 production. In CA-SMCs, the inflammatory stimulation did not increase STAT3 abundance but increased STAT1 and STAT2 abundance at the 24 and 48 time point. CA-SMCs responded to the inflammatory stimulation by increasing STAT1, 2, and 3 activation acutely (5 h) and at prolonged stimulation time points (24 and 48 h). The IMPACs-CM did not have noticeable effect on the CA-SMC STATs abundance and activation state.

STAT4, another member of STAT family of transcription factors was also analyzed. The inflammatory treatment did not contain STAT4 activators, consequently, an increase in the STAT4 activation was not observed; however, STAT4 was increased in abundance in these cells. The IMPACs-CM treatment reduced the STAT4 abundance in macrophages at the baseline and 48 h post inflammatory treatment.

STAT5 is activated by IL-2, IL-3, IL-7, GM-CSF, growth factors, and hormones and plays a critical role in the development and function of many cell types (20). STAT5 protein expression was detected in macrophages, CA-ECs, CA-SMCs, and fibroblast cells. The inflammatory treatment most prominently increased STAT5 in macrophages and CA-ECs at 5 and 48 h stimulation time point. The IMPACs-CM suppressed the increase in STAT5 at these time points. The fibroblast cells exhibited the same increase and the suppression only at 5 h time point. The CA-SMCs responded to the inflammatory stimulation opposite way reducing STAT5 protein expression at 24 and 48 h. In these cells the IMPACs-CM did not exhibit the suppression effect.

STAT6 is activated by IL-4 and IL-1β and plays a major role in the immune system (21). STAT6 is involved in T cell and B cell proliferation, Th2 polarization (22, 23). In macrophages, STAT6 is involved in polarization of M2 macrophage that exhibit anti-inflammatory functions (24, 25). Additionally, there is increasing evidence that STAT6 may have important roles in development of non-immune cells related disease processes (21). STAT6 in the cells that were tested responded to the inflammatory stimulation and the IMPACs-CM treatment very similar to the responses of STAT5.

IMPACs Secretomes Increase Production of Active TGFb in Monocytes and Cardiac Myocytes (FIG. 9D): The presence or absence of IL-6 determines how T cells respond to TGFβ. TGFβ induces differentiation of both pro-inflammatory Th17 cells and the FoxP3⁺ regulatory T cells (Tregs). IL-6 induces the differentiation of Th17 cells from naïve T cells. At the same time IL-6 inhibits the differentiation of Treg (26). Two forms of TGFβ were detected: latent inactive (44 kD) and cleaved active form (12.5 kD) in monocytes and neonatal rat ventricular myocytes (NRVMs). IMPACs-CM treatment induced an increase in the active form of TGFβ in both monocytes and NRVMs. Inflammatory stimulation also increased the active TGFβ in the NRVMs when stimulated for prolonged times (24 and 48 h). These results indicated that IMPACs paracrine factors could induce production of the active form of TGFβ in cardiac myocytes and recruited monocytes. In addition, the IMPACs themselves secrete TGFβ. At the same time, the IMPACs paracrine factors inhibit IL-6 production as was observed in hPBDMs, monocytes, and CA-ECs. Collectively, the differentiation of Th17 cells would be inhibited and Treg differentiation would be promoted by factors secreted by IMPACs; thereby shifting the balance between Th17 (inflammatory) and Treg (regulatory) toward the toward the resolution of inflammation.

IMPACs paracrine factors promotes homeostasis of paracrine factors/cytokines to augment wound healing (FIGS. 9E, 9F and 9G): The myocardium contains many types of cells that uniquely respond to inflammatory stimuli differently. The sum of these responses determines overall wound healing after MI. To better understand the effect of the IMPACs-CM in the injured heart, two immune cells were studied; hPBDMs and human peripheral blood derived monocytes (monocytes) because these cells are recruited to the injured myocardium. Four major myocardium cells were also studied: cardiac myocyte (neonatal rat ventricular myocytes: NRVMs), coronary artery endothelial (CA-ECs), cardiac fibroblast (fibroblast), and coronary artery smooth muscle cells (CA-SMCs). The pro-inflammatory cytokine, IL-6 levels were measured in response to IMPACs-CM during inflammatory stimulation. Without the inflammatory stimulation, IL-6 was not detectable in these cells except for in the fibroblast. Five hours after the inflammatory stimulation, acute production of IL-6 was detected in all of the cells except for the NRVMs. The IMPACs-CM treatment reduced the productions of IL-6 in the hPBDMs, monocytes, and CA-ECs. IL-6 production in the hPBDMs was significantly lower and almost undetectable after the acute production at 5 h. The other cell types kept responding to the inflammatory stimulation, producing IL-6 until the end of 48 h study. The IMPACs-CM treatment had a long-lasting suppression on IL-6 production in the endothelial cells. The acute production and suppression of IL-6 in macrophages were especially large compared to other cells indicating that the hPBDMs provide the early increase in IL-6 during acute inflammation and the IMPACs paracrine factors suppress that increase. IL-6 is the master stimulator of the most acute phase proteins (27). Immediately after tissue injuries, elevated levels of IL-6 production and secretion were observed and found to promote inflammation by inducing recruitment and differentiation of pro-inflammatory leukocytes (28). Additionally, prolonged IL-6 secretion and its soluble IL-6 receptor complex play an important role in the transition from acute to chronic inflammation. The hypothesis that IMPACs secreted paracrine factors in IMPACs-CM reduce IL-6 production to suppress excess activation during the acute phase of inflammation to protect hearts after MI, was tested. All of the cells that were tested, except for NRVMs, increased IL-6 production with the inflammatory treatment. Inflammatory treatment also strongly stimulated macrophage production of IL-6, but only at 5 h. All of the other cell types elevated their IL-6 production for the duration of exposure to the inflammatory environment. IMPACs-CM prominently suppressed IL-6 production in the two immune cells we tested at 5 h. This effect was not as seen for the CA-ECs; however, the effect to reduce IL-6 was observed not only at 5 h but also at 24 and 48 h. Prolonged increases in systematic IL-6 in rats using osmotic pump induced myocardial hypertrophy, increase in ventricular stiffness, and myocardial fibrosis (29). Inhibition of IL-6 signal transduction was found to be beneficial in MI damaged heart. Specifically, IL-6 receptor antibody infusion blocked IL-6 signal transduction and reduced neutrophil and macrophage infiltration to attenuate left ventricular remodeling and increased the post MI survival rate (30). Therefore, the data herein support the hypothesis that IMPACs injected into the heart after MI secrete factors that suppress IL-6 secretion, modifying the immune response and leading to improved cardiac structure and function.

Additionally, IMPACs-CM effect on production of the NF-κB target gene, IL-8 was also studied. The IMPACs-CM reduced the expression of IL-8. hPBDMs exhibited only acute production of IL-8 after treatment with inflammatory agents while the endothelial cells exhibited prolonged production. IMPACs-CM suppressed IL-8 productions under these conditions. IL-8 is used as a marker of cardiovascular disease (31). The inflammatory treatment elevated IL-8 production in CA-ECs at every time point and the IMPACs-CM inhibited this IL-8 production at 24 and 48 h. In hPBDMs, the inflammatory treatment also elevated the IL-8 production and the IMPACs-CM inhibited this production in macrophages; however, these phenomena were observed only at 5 h time point. These results, therefore, provide evidence that the IMPACs-CM contain secreted factors with the potential to inhibit excessive recruitment of immune cells to a site of inflammation.

The chemokine receptor, CXCR4 is a G protein coupled receptor. Upon activation with CXCR12 binding, CXCR4 is phosphorylated by PKC and multiple G protein-coupled receptor kinases and regulated in both positive and negative modulation of CXCR4 signaling (32). The CXCR4 activation is best known to be important for hematopoietic cell homing and controlling the quiescence of primitive hematopoietic cells (33). In a non-inflammatory environment, the IMPACs-CM induced significant expression of CXCR4 within human monocytes and hPBDMs. With inflammatory treatment there was still a small increase in CXCR4 in these cells. IMPACs-CM induced increases in CXCR4 were not seen under inflammatory conditions. Additionally, the CXCR4 detected under the non-inflammatory environment exhibited very high level of phosphorylation ladder indicating that CXCL12 contained in the IMPACs-CM activates CXCR4 signaling pathways (CXCL12 ELISA needed). The small amount of CXCR4 detected under inflammatory conditions did not exhibit the prominent phosphorylation ladder indicating that the CXCl12 activation of CXCR4 was inhibited. Reductions in CXCL12 secretion from the IMPACs during inflammatory stimulation might have caused the reduced CXCR4 activation. Inflammatory stimulations via Toll like receptor (TLR) pathway induce increase in surface expression of CXCR4(34). Moreover, the CXCR4 expression was reported to be down-regulated by cytokines interleukin-4 (IL-4), IL-13, and granulocyte-macrophage colony-stimulating factor (GM-CSF) and up-regulated by IL-10 and transforming growth factor-p1 (35). Under inflammatory stimulation, IMPACs express IL-13, IL-10, and TGFb (Western). What was observed was combined effect of unidentified factors in the conditioned medium. hPBDMs augmented CXCR4 expression in inflammatory media; while, IMPACs-CM treated hPBDMs did not exhibit VEGF and Tie2 expressing angiogenic macrophage phenotype even after 48 h of incubation. These data strongly demonstrate the role of IMPACs in balancing the cytokines after injury.

### DISCUSSION

The present studies report characterizing primary isolation of IMPACs phenotypes from bone fragments obtained at the time of surgeries. IMPACs have demonstrated to mediate wound healing processes by targeting multiple cardiac cell types and immune cells. Additionally, IMPACs modify the cytokine/paracrine landscape to a pro reparative type that augment wound healing processes after injury. It was shown that isolated IMPACs were highly cloneable and were able to grow without senescence in our growth medium. At the higher passages, the IMPACs grew faster to the doubling hour of nearly 10 hours. Additionally, IMPACs had a unique surface profile, exhibiting very few surface antigen expressions. IMPACs were negative for characteristic markers of CSCs, MSCs, hematopoietic lineage markers. Also, IMPACs showed a different marker profile compared to CBSCs isolated from other mammalian species by the inventor's group (1, 36). IMPACs abundantly expressed a common stem cell antigen CD49f (5) and embryonic surface antigens (37-39) signifying their primitive nature. IMPACs were negative for “self-antigen”, MHC-Ia. Like other types of CBSCs, IMPACs did not express HLA-DR; however, the IMPACs were found to express HLA-DQ another subtype of MHC-II indicating that the IMPACs have some property of antigen presenting cells.

It was demonstrated that in a small and large animal model that CBSCs transplantation improved cardiac function. It was also tested whether CIMPACT treatment post-I/R can also augment cardiac function. CIMPACT largely preserved the contractility of the heart with less reduction in ejection fraction and preserved myocardium synchronous compared to mice treated with hECs and PBS. Additionally, CIMPACT transplanted animals showed significantly smaller infarct sizes. Despite the functional benefit and remodeling of heart after injury, no transdifferentiation of the transplanted cells was observed, meriting exploration of paracrine mechanisms responsible for functional and structural improvement.

IMPACs have a potential applicability for allogeneic cell therapy (1, 2, 36) due to its enhanced wound healing capabilities. There is a consensus in the field that cell types that can engraft and evade the host immune cells will be a suitable candidate for cell therapy. Interestingly, it was found that the IMPACs do not express the self-antigen, indicating that the cytotoxic T cells cannot recognize the injected IMPACs. However, NK cells can recognize the IMPACs therefore it was tested if hCBSCs are equipped to defend against the NK cells and it was found that IMPACs have ability to evade hNK cells. IMPACs secretome is the major source of the inhibition of hNK cell cytotoxicity activity. IMPACs produce soluble forms of HLA-Gs and numerous other anti-inflammatory factors including IL-4, IL-10, IL-35, IL-IRA, and TGFβ that could support IMPACs evading from the host immune cells. To test whether IMPACs can escape the immune response and engraft IMPACs were injected in WTC57/BL6 mice and observed that IMPACs can survive 6-weeks post injection. IMPACs transplantation in mice after I/R also significantly improved their cardiac function demonstrating their enhanced wound healing abilities. that is much longer than previously observed (40). Taken together these studies provide evidence that IMPACs are more likely not to be rejected by host immune system if injected into human myocardium.

Myocardial infarction induced death of myocardial cells release danger signaling molecules activating an intense inflammatory response by immune cells and myocardial cells including cardiomyocytes, fibroblast, and endothelial cells. These inflammatory cells synthesize pro-inflammatory cytokines and chemokines and promote recruitment of leukocytes (41). Excessive leukocyte recruitment and infiltration produces a large amount of reactive oxygen species and causes extension of ischemic injury and expand infarct area (42). Injected IMPACs were thought to produce paracrine factors and suppress these chains of reactions, preserve myocardial cells, and support the regenerative processes (43). Consequently, the major focus of the present study was to explore immune modulatory effect of IMPACs secretome. To evaluate the immune modulatory effect of IMPACs secretome, pro-inflammatory molecules IL-6 and IL-8 were targeted to see if the IMPACs secretions modify the production of these molecules. IL-6 is the master stimulator of the most acute phase proteins (27). Immediately after tissue injuries, elevated levels of IL-6 production and secretion have been observed and found to promote inflammation by inducing recruitment and differentiation of pro-inflammatory leukocytes (28). Additionally, prolonged IL-6 secretion and its soluble IL-6 receptor complex play important role in transition from acute to chronic inflammation. IL-8 is used as a marker of cardiovascular disease (31). The primary role of IL-8 in acute inflammation is in the recruitment of neutrophils (44). It is also responsible for the chemotactic migration and activation of monocytes, lymphocytes, basophils, and eosinophils at sites of inflammation (45). The inflammatory treatment described herein, sharply increased both IL-6 and IL-8 in hPBDMs and monocytes.IMPACs secretome almost completely suppressed the inflammation induced IL-6 and IL-8 production. These results, therefore, indicate that the IMPACs secretome have potential to inhibit excessive production of inflammatory cytokines and chemokines and suppress recruitment of immune cells to the site of inflammation. Beyond the effect of IMPACs secretome on the leukocytes, it was found that the IMPACs secretome exert a potential anti-inflammatory effect on T-lymphocytes. The differentiation of pro-inflammatory Th17 cell and FoxP3⁺ regulatory cell (Treg) is dependent on balance between IL-6 and TGFβ (15). TGFβ drives differentiation of both Th17 and Treg. IMPACs produce only latent inactive form of TGFβ but the IMPACs secretome has the ability to influence neighboring cells such as monocytes and cardiac myocytes to produce active form of TGFβ.

During the inflammation phase of wound healing, increased levels of reactive oxygen species and IL-1 stimulate cardiac fibroblasts to acquire a pro-inflammatory phenotype. Suppression of this inflammatory response can induce a reparative myofibroblast phenotype that preserves myocardial integrity and prevents subsequent ventricular dilation and failure. The present studies showed that IMPACs secrete at least three molecules TSP-1, TGFβ, and IL-1RA that can induce changes in fibroblast phenotypes that promote the reparative phases of the wound healing response. After MI, IL-1 is secreted from the injured cells and infiltrating immune cells and this promotes a proinflammatory environment. IL-1RA is a member of IL-1 family of cytokines that is a natural antagonist of IL-1 signaling. The present results show that IMPACs synthesize small amount of IL-iRA constitutively and, when IMPACs are placed in an inflammatory environment IL-R1A synthesis is increased. When the stimulation is persistent IMPACs further augmented IL-iRA synthesis. IL-1RA has been reported to have a cardioprotective role suppressing cardiac myocyte apoptosis after MI.

IL-1RA secretion by IMPACs may be a crucial molecule to modify the inflammatory microenvironment so that TSP/TGFβ can function to promote wound healing. Activation of IL-1 signaling in cardiac fibroblasts inhibits TGFβ dependent αSMA expression resulting in a delay of myofibroblast conversion. IL-1 signaling also promotes a matrix degrading fibroblast phenotype and delays a collagen producing synthetic myofibroblast phenotype. Additionally, IL-1 stimulation also downregulates TSP-1 expression in fibroblasts and further deters effects downstream to TGFβ. The present results suggest that IL-1 signal inhibition by IL-1RA, secreted by IMPAC, promotes wound healing through TSP-1/TGFβ signaling cascades.

IMPAC secreted TSP-1 into conditioned medium. TSP-1 is a matricellular glycoprotein bound to the ECM. It was detected in areas of the damaged heart where IMPACs were injected. TSP-1 binds to the latent inactive TGFβ complex and thereby induces a biologically active form of TGFβ. In an inflammatory environment, TSP-1/TGFβ signaling can play a cardioprotective role by suppressing proliferation of inflammatory T lymphocyte, inducing regulatory T cell differentiation, and inhibiting inflammatory macrophages, thereby leading to markedly reduced cytokine and chemokine synthesis and decreasing reactive oxygen generation. TSP-1/TGFβ signaling can be a master switch, regulating the transition from an inflammatory to reparative phase of wound healing. TSP-1 also binds to CD36 scavenger receptor of macrophages and activates their phagocytosis and shifts macrophages toward a reparative phenotype. The phagocytotic macrophages in turn increase secretion of anti-inflammatory factors, such as TGFβ and IL-10. The reparative macrophages then secrete growth factors that enhance the repair. TSP-1 and TGFβ are also can influence fibroblast phenotypes. These molecules are involved in promotion of myofibroblast differentiation, leading to ECM synthesis, and ECM preservation. Endogenous fibroblasts of IMPAC injected myocardium exhibited early myofibroblast differentiation and withdrawal indicating that IMPACs may promote a more rapid form of wound healing. TSP-1 expression is also upregulated by hypoxia through hypoxia induced factors, HIF1α and HIF1β. This mechanism is beneficial for a cell therapy targeting MI since the cells are likely to function in a low oxygen environment. TSP-1 null mice exhibit defective TGFβ signaling and impaired myofibroblast differentiation and collagen expression in the myocardium following pressure overload [ ]. The present data suggests that the TSP-1 secreted by IMPACs may be a critical factor promoting the prohealing effects of these cells.

Myocardial ECM integrity is largely dependent upon MMP family of endopeptidases that regulate balance between the ECM synthesis and degradation. The present studies showed that IMPAC-secreted TSP-1 in the MI border zone attenuates the inflammatory stimulation induced MMP3 production in cardiac fibroblast cells. As discussed above, TSP-1 is an activator of TGFβ and TGFβ is an endogenous inhibitor of MMP3. Thus, the results herein, support the idea that TSP-1 secreted from injected IMPACs have potential stabilizing effects on the post MI myocardium by both increasing ECM synthesis and suppressing its degradation. After MI, MMP3 expression and activity increase. The plasma concentration of MMP3 is an indicator of post MI prognosis that correlates with adverse post MI remodeling, impaired function, and a greater risk of heart failure or death. IMPAC-induced inhibition of MMPs could attenuate post MI remodeling and to improve cardiac structure and function.

Another axis that IMPACs targeted was CXCR4. IMPACs condition medium induced huge increase in CXCR4 production and a lesser extent in monocytes. The condition medium induced CXCR4 expression was associated with their phosphorylation ladder implicating that the CXCR4 responding to CXCR12 contained in the condition. The condition medium induced CXCR4 protein expression was limited only at the baseline. The inflammatory stimulation abolished the condition medium induced CXCR4 production. CXCR4 expression has been reported to be reduced by IL-4 and increased by IL-10 and TGFβ. IMPACs express all of these molecules; however, the expression of IL-10 and TGFβ might have overrode the IL-4 suppression and possibly increased CXCR4 expression. Increased CXCR4 has been observed in aged neutrophils homing toward bone marrow (46) and transitional precursor monocyte subpopulation (47). At this point, it was thought that either the condition medium treated macrophages and monocytes aged or de-differentiated toward the monocyte precursor. Nevertheless, the condition medium treated macrophage exhibit morphology more like a monocyte than a macrophage and have reduced M1 like morphological phenotype and response to the inflammatory stimulation.

Using the wound healing assay format, it was shown that IMPACs secrete factors that modify fibroblast and endothelial cell growth. Both cells grew well in the base growth medium but responded to IMPACs condition medium in a completely opposite way in that the growth of fibroblast cells was accelerated while the same condition medium suppressed the growth of endothelial cells indicating that the condition medium was giving the cell specific instruction to these cells. These modulations were largely reversed with the inhibition of exosome production indicating that the exosome is one of the most important IMPACs secretome. Exosome secreted from CBSCs have been shown to regenerate tissue through miRNA (48, 49). Although only few of key immune modulatory molecules were detected with Western blot analysis, the IMPACs protein production is few and very low compared to hMSCs as discussed above. Hence, study was focused on miRNA contained in IMPACs exosomes. The analysis revealed that the exosomes contain miRNAs that target proteins of two opposing sides of their functions and provide bi-directional instruction to a target cell. A representative example was seen in the NRVM experiment. The IMPACs co-culture exerted a protective effect on NRVMs against a high level of oxidative stress increasing their survival rate, preserving a known survival molecule Akt and its activation. Simultaneously, NRVMs without the oxidative stress in the growth environment actually reduced Akt and its activation. The IMPACs exosomes have enriched miRNAs that were reported to function both cell growth and suppression. More specifically, the IMPACs exosomes contained miRNAs that target PI3K/Akt pathway molecules that promote Akt activation, Akt2, PI3K and PDK1 and also PTEN that suppress Akt activation by dephosphorylating PIP3, a product of PI3K. Another evidence was seen in macrophage that the explosive production of cytokine and chemokine and also transcription factors in response to the inflammatory stimulation were prominently reduced with IMPACs condition medium. The condition medium did very little to the non-stimulated counterpart. The interpretation of these effects was that the IMPACs secretome (miRNAs) acts to keep cellular homeostasis. In case of overgrowth or stimulation induced protein production, the IMPACs secretome (miRNAs) acts to suppress the over expressed protein production to less stressful resting level for each cell type. If the target cells were facing a death threatening environment, another set of the secretome (miRNA) activate cell survival molecule in support of the stressed cells. The mechanism of these effects may be that transduced miRNA have a higher chance of binding to the genes over expressed and suppress those genes.

### REFERENCES

1. Duran, J. M., et al., Bone-derived stem cells repair the heart after myocardial infarction through transdifferentiation and paracrine signaling mechanisms. Circ Res, 2013. 113(5): p. 539-52.
2. Sharp, T. E., 3rd, et al., Cortical Bone Stem Cell Therapy Preserves Cardiac Structure and Function After Myocardial Infarction. Circ Res, 2017. 121(11): p. 1263-1278.
3. Kambayashi, T. and T. M. Laufer, Atypical MHC class II-expressing antigen-presenting cells: can anything replace a dendritic cell?Nat Rev Immunol, 2014. 14(11): p. 719-30.
4. Yu, K. R., et al., CD49f enhances multipotency and maintains stemness through the direct regulation of OCT4 and SOX2. Stem Cells, 2012. 30(5): p. 876-87.
5. Krebsbach, P. H. and L. G. Villa-Diaz, The Role of Integrin alpha6 (CD49f) in Stem Cells: More than a Conserved Biomarker. Stem Cells Dev, 2017. 26(15): p. 1090-1099.
6. Dittmer, J. and B. Leyh, Paracrine effects of stem cells in wound healing and cancer progression (Review). Int J Oncol, 2014. 44(6): p. 1789-98.
7. Chen, Y. and X. Wang, miRDB: an online database for prediction of functional microRNA targets. Nucleic Acids Res, 2020. 48(D1): p. D127-D131.
8. Liu, W. and X. Wang, Prediction of functional microRNA targets by integrative modeling of microRNA binding and target expression data. Genome Biol, 2019. 20(1): p. 18.
9. Vaure, C. and Y. Liu, A comparative review of toll-like receptor 4 expression and functionality in different animal species. Front Immunol, 2014. 5: p. 316.
10. Pahl, H. L., Activators and target genes of Rel/NF-kappaB transcription factors. Oncogene, 1999. 18(49): p. 6853-66.
11. Pye, J., et al., Proteasome inhibition ablates activation of NF-kappa B in myocardial reperfusion and reduces reperfusion injury. Am J Physiol Heart Circ Physiol, 2003. 284(3): p. H919-26.
12. Frantz, S., et al., Absence of NF-kappaB subunit p50 improves heart failure after myocardial infarction. FASEB J, 2006. 20(11): p. 1918-20.
13. Moss, N.C., et al., IKKbeta inhibition attenuates myocardial injury and dysfunction following acute ischemia-reperfusion injury. Am J Physiol Heart Circ Physiol, 2007. 293(4): p. H2248-53.
14. Bartekova, M., et al., Role of cytokines and inflammation in heart function during health and disease. Heart Fail Rev, 2018. 23(5): p. 733-758.
15. Tanaka, T., M. Narazaki, and T. Kishimoto, IL-6 in inflammation, immunity, and disease. Cold Spring Harb Perspect Biol, 2014. 6(10): p. a016295.
16. Laouar, Y., et al., STAT3 is required for Flt3L-dependent dendritic cell differentiation. Immunity, 2003. 19(6): p. 903-12.
17. Takeda, K., et al., Enhanced Th1 activity and development of chronic enterocolitis in mice devoid of Stat3 in macrophages and neutrophils. Immunity, 1999. 10(1): p. 39-49.
18. Panopoulos, A. D., et al., STAT3 governs distinct pathways in emergency granulopoiesis and mature neutrophils. Blood, 2006. 108(12): p. 3682-90.
19. Yang, X. O., et al., STAT3 regulates cytokine-mediated generation of inflammatory helper T cells. J Biol Chem, 2007. 282(13): p. 9358-63.
20. Owen, D. L. and M. A. Farrar, STATS and CD4 (+) T Cell Immunity. F1000Res, 2017. 6: p. 32.
21. Goenka, S. and M. H. Kaplan, Transcriptional regulation by STAT6. Immunol Res, 2011. 50(1): p. 87-96.
22. Takeda, K., et al., Essential role of Stat6 in IL-4 signalling. Nature, 1996. 380(6575): p. 627-30.
23. Kaplan, M. H., et al., Stat6 is required for mediating responses to IL-4 and for development of Th2 cells. Immunity, 1996. 4(3): p. 313-9.
24. Stein, M., et al., Interleukin 4 potently enhances murine macrophage mannose receptor activity: a marker of alternative immunologic macrophage activation. J Exp Med, 1992. 176(1): p. 287-92.
25. Martinez, F. O., et al., Genetic programs expressed in resting and IL-4 alternatively activated mouse and human macrophages: similarities and differences. Blood, 2013. 121(9): p. e57-69.
26. Bettelli, E., et al., Reciprocal developmental pathways for the generation of pathogenic effector TH17 and regulatory T cells. Nature, 2006. 441(7090): p. 235-8.
27. Gabay, C., Interleukin-6 and chronic inflammation. Arthritis Res Ther, 2006. 8 Suppl 2: p. S3.
28. Youker, K., et al., Neutrophil adherence to isolated adult cardiac myocytes. Induction by cardiac lymph collected during ischemia and reperfusion. J Clin Invest, 1992. 89(2): p. 602-9.
29. Melendez, G. C., et al., Interleukin 6 mediates myocardial fibrosis, concentric hypertrophy, and diastolic dysfunction in rats. Hypertension, 2010. 56(2): p. 225-31.
30. Kobara, M., et al., Antibody against interleukin-6 receptor attenuates left ventricular remodelling after myocardial infarction in mice. Cardiovasc Res, 2010. 87(3): p. 424-30.
31. Dominguez-Rodriguez, A., et al., Prognostic value of interleukin-8 as a predictor of heart failure in patients with myocardial infarction and percutaneous intervention. Int J Cardiol, 2006. 111(1): p. 158-60.
32. Busillo, J. M., et al., Site-specific phosphorylation of CXCR4 is dynamically regulated by multiple kinases and results in differential modulation of CXCR4 signaling. J Biol Chem, 2010. 285(10): p. 7805-17.
33. Nie, Y., Y. C. Han, and Y. R. Zou, CXCR4 is required for the quiescence of primitive hematopoietic cells. J Exp Med, 2008. 205(4): p. 777-83.
34. Petty, J. M., et al., Pulmonary stromal-derived factor-1 expression and effect on neutrophil recruitment during acute lung injury. J Immunol, 2007. 178(12): p. 8148-57.
35. Wang, J., et al., Role of tyrosine phosphorylation in ligand-independent sequestration of CXCR4 in human primary monocytes-macrophages. J Biol Chem, 2001. 276(52): p. 49236-43.
36. Mohsin, S., et al., Unique Features of Cortical Bone Stem Cells Associated With Repair of the Injured Heart. Circ Res, 2015. 117(12): p. 1024-33.
37. Henderson, J. K., et al., Preimplantation human embryos and embryonic stem cells show comparable expression of stage-specific embryonic antigens. Stem Cells, 2002. 20(4): p. 329-37.
38. Tilgner, K., et al., Isolation of primordial germ cells from differentiating human embryonic stem cells. Stem Cells, 2008. 26(12): p. 3075-85.
39. Schopperle, W. M. and W. C. DeWolf, The TRA-1-60 and TRA-1-81 human pluripotent stem cell markers are expressed on podocalyxin in embryonal carcinoma. Stem Cells, 2007. 25(3): p. 723-30.
40. Kinnaird, T., et al., Local delivery of marrow-derived stromal cells augments collateral perfusion through paracrine mechanisms. Circulation, 2004. 109(12): p. 1543-9.
41. Saxena, A., I. Russo, and N. G. Frangogiannis, Inflammation as a therapeutic target in myocardial infarction: learning from past failures to meet future challenges. Transl Res, 2016. 167(1): p. 152-66.
42. Entman, M. L., et al., Neutrophil induced oxidative injury of cardiac myocytes. A compartmented system requiring CD11b/CD18-ICAM-1 adherence. J Clin Invest, 1992. 90(4): p. 1335-45.
43. Hodgkinson, C. P., et al., Emerging Concepts in Paracrine Mechanisms in Regenerative Cardiovascular Medicine and Biology. Circ Res, 2016. 118(1): p. 95-107.
44. Hammond, M. E., et al., IL-8 induces neutrophil chemotaxis predominantly via type I IL-8 receptors. J Immunol, 1995. 155(3): p. 1428-33.
45. Miller, E. J., et al., Elevated levels of NAP-1/interleukin-8 are present in the airspaces of patients with the adult respiratory distress syndrome and are associated with increased mortality. Am Rev Respir Dis, 1992. 146(2): p. 427-32.
46. Martin, C., et al., Chemokines acting via CXCR2 and CXCR4 control the release of neutrophils from the bone marrow and their return following senescence. Immunity, 2003. 19(4): p. 583-93.
47. Chong, S. Z., et al., CXCR4 identifies transitional bone marrow premonocytes that replenish the mature monocyte pool for peripheral responses. J Exp Med, 2016. 213(11): p. 2293-2314.
48. Katsuda, T. and T. Ochiya, Molecular signatures of mesenchymal stem cell-derived extracellular vesicle-mediated tissue repair. Stem Cell Res Ther, 2015. 6: p. 212.
49. Ferguson, S. W., et al., The microRNA regulatory landscape of MSC-derived exosomes: a systems view. Sci Rep, 2018. 8(1): p. 1419.

### Example 2: Effects of IMPACs Medium on IMPACs and hMSC Growth Characteristics

To determine if the IMPACs medium converts hMSCs to IMPACs, IMPACs medium induced growth characteristics IMPACs phenotypes (1.0-3.0) and hBMSCs/hMSCs (hMSCs) were compared at a lower and higher passage number using a single cell clone technique. The post isolation passage number 4 (P4) was chosen as the lower passage for all of the cells. The passage number 10 (P10) and 80 (P80) as the high passage number for hMSCs and IMPACs phenotypes respectively.

### Results:

Cell shape and size: Cloned IMPACs were small, round shaped, and were mixed with spindle shaped cells. The hMSCs were flatter and much larger than IMPACs.

Clonability: 100% of the low and high passage IMPACs grew and formed large and dense colonies. The P80 IMPACs had lower doubling time (between Day 0 to 3) compared to the P4 counter parts. All of the low passage hMSCs (P4) plated divided at least once during this 7-day experimental period; however, the mean doubling time between the Day 0 to 3, for this group was 66 h that is nearly 5-fold longer than the doubling time of P4 IMPACs. Nine high passage hMSC (P10) plated were not cloneable at all. These cells quickly senesced and did not divide.

TABLE 5
IMPAC Mean Cell Count and Doubling Hours

| Cell Type | Passage# | n | Day 0 | Day 1 | Day 2 | Day 3 | Doubling (h) |
|---|---|---|---|---|---|---|---|
| IMPAC 1.0 | 4* | 8 | 1 | 4.38 | 15.75 | 58.00 | 13.20 |
| IMPAC 2.0 | 4* | 7 | 1 | 3.29 | 12.86 | 48.86 | 13.00 |
| IMPAC 3.0 | 4* | 8 | 1 | 2.63 | 9.13 | 34.75 | 14.69 |
| IMPAC 1.0 | 80 | 8 | 1 | 2.88 | 20.29 | 109.25 | 11.53 |
| IMPAC 2.0 | 80 | 8 | 1 | 3.75 | 25.25 | 128.50 | 10.82 |
| IMPAC 3.0 | 80 | 6 | 1 | 3.67 | 18.67 | 69.50 | 12.51 |

Notes:
*indicates passage number adjusted to the post isolation passage. Doubling time was calculated between Day 0 and 3.

hBMSC Mean Cell Count and Doubling Hours

| Cell Type | Passage# | n | Day 0 | Day 1 | Day 2 | Day 3 | Doubling (h) |
|---|---|---|---|---|---|---|---|
| hBMSC | 4* | 6 | 1 | 1.00 | 1.17 | 2.33 | 66.00 |

Notes:
*indicates passage number adjusted to the post isolation passage. Doubling time was calculated between Day 0 and 3.

The high passage hBMSC (n=9) data is not listed here because none of the cells divided; thus, no doubling time was derived.

### Conclusion:

The hMSCs grown in the IMPACs medium has totally different morphology, growth, and senescence characteristics compared to the IMPACs. Conversion of IMPACs from hMSCs was not observed. Thus, the IMPACs medium does not convert hMSCs to IMPACs.

### Example 3: Effect of Culture Media on hMSC Surface Marker Characteristics

The Mesenchymal and Tissue Stem Cell Committee of the International Society for Cellular Therapy proposed minimal criteria to define human MSC (hMSC) are:
1) hMSC must be plastic-adherent when maintained in standard culture conditions;
2) Express CD105, CD73 and CD90 (>95%);
3) Lack surface expression of CD45, CD34, CD14 or CD11b, CD79a or CD19 and HLA-DR (<2%);
4) Differentiate to osteoblasts, adipocytes and chondroblasts in vitro (Not tested in the present study);
5) Additionally, hMSCs express HLA-ABC.

The isolated IMPACs were found to express none of these markers when cultured with IMPACs growth medium. To determine if the IMPACs growth medium convert hMSCs to the IMPACs, low passage (P4) and high passage (P10) hMSCs were cultured under influence of hMSC (10% FBS aMEM) or IMPACs medium and analyzed for the growth progression and cell surface marker characteristics.

The low passage hMSCs cultured with hMSC medium met the hMSC surface expression criteria listed above. The rate of CD73 and CD90 positive cells though were lower than the proposed criteria. At the high passage, in addition to the positive markers detected for the low passage, every other markers that were tested, HLA-DQ, hematopoietic progenitors (CD34), pan leukocytes (CD45), monocytes (CD14 and CD11b), B cell (CD19) became positive, indicating that differentiation of the hMSCs may have taken place.

The low passage hMSCs cultured with IMPACs medium had similar tendencies that were observed with the hMSC medium. In addition to that tendency, minute increases in the hematopoietic progenitor and the monocytes marker was detected. HLA-DR expression that was not expressed with the hMSC medium culture was detected. At the high passage, these hematopoietic lineage marker levels were augmented.

The results indicated that the IMPACs medium did not induce the IMPACs surface marker characteristics in the hMSC under any of the conditions tested. Thus, it was concluded that the IMPACs medium does not convert hMSCs to IMPACs.

TABLE 6
Effect of Repeated Passages on Surface Marker Characteristics of IMPACs Phenotypes

| | IMPAC 1.0 | IMPAC 1.0 | IMPAC 1.0 | IMPAC 1.0 | IMPAC 1.0 | IMPAC 1.0 |
|---|---|---|---|---|---|---|
| | P4* | P4* | P4* | P100 | P100 | P100 |
| CD73 | 0.36% | 0.88% | 0.84% | 1.32% | 1.52% | 2.26% |
| CD90 | 0.81% | 1.61% | 2.02% | 0.53% | 1.72% | 0.47% |
| CD105 | 0.84% | 1.06% | 1.24% | 0.52% | 2.55% | 0.54% |
| CD34 | 0.96% | 1.82% | 1.81% | 0.28% | 21.10% | 1.36% |
| CD45 | 0.74% | 3.03% | 2.95% | 1.92% | 3.14% | 2.19% |
| CD19 | 0.30% | 2.17% | 2.02% | 0.65% | 3.34% | 0.68% |
| CD14 | 0.35% | 0.21% | 0.21% | 0.82% | 14.00% | 2.73% |
| CD11b | 0.64% | 0.14% | 0.14% | 0.09% | 16.70% | 0.94% |
| HLA-DQ | 20.20% | 41.50% | 16.80% | 0.75% | 9.88% | 0.47% |
| HLA-ABC | 1.09% | 2.84% | 2.39% | 0.50% | 50.90% | 4.39% |
| HLA-DR | 0.20% | 0.58% | 0.18% | 1.11% | 20.70% | 3.21% |

Notes:
*indicates passage number adjusted to the post isolation passage.

### Example 4: CBSC Special Diversity in Surface Marker Expression

To study special diversity in CBSCs, human, mouse, pig CBSCs and human bone marrow stromal cells (BMSCs/hMSCs) to post-isolation passage 4 (P4) were cultured. The mouse CBSCs (mCBSCs) and pig CBSCs (sCBSCs) were cultured with mCBSC medium. The IMPACs phenotypes (1.0-3.0) were cultured with IMPACs medium. Bone marrow stromal cells (BMSCs) or hMSCs were also cultured with IMPACs medium and used as the reference cells. Each of the cell types were then analyzed for 242 human, 178 mouse, and 6 pig surface markers using flow cytometry for their comparison. The number of pig marker analysis was limited because of the availability of established pig surface marker antibodies.

### Results:

The IMPACs did not express many surface markers and the markers expressed were different from those expressed by BMSCs/hMSC, mCBSC, and sCBSCs. The IMPACs and mCBSCs both expressed high levels of CD49f. The CD49f (integrin alpha chain alpha 6) is the only stem cell marker commonly found in more than 30 stem cells. The IMPACs and mCBSCs expressed SSEA1, one of the embryonic stem cell markers. The expression levels were variable (SCBSCs: 2.51%; MCBSCs: 25.5%; LCBSCs: 72.80%; and mCBSCs: 55.8%). The IMPACs and mCBSCs along with hMSCs expressed SSEA4, another embryonic stem cell marker. The expression levels were variable (IMPAC 1.0: 0.83%; IMPAC 2.0: 27.4%; IMPAC 3.0: 2.69%; mCBSCs: 22.00%, and hMSC: 36.7%). CD73, known hMSC marker was highly positive in hMSCs and mCBSCs but negative in IMPACs phenotypes. CD90, a known hMSC marker was positive in hMSCs but negative for mCBSCs and hCBSC phenotypes.CD105, a known hMSC marker was highly positive in hMSCs and mCBSCs but negative in sCBSCs and hCBSC phenotypes. CD29, a known hMSC marker was highly positive in hMSCs, mCBSCs, and sCBSCs but negative in IMPACs phenotypes. CD44, a known hMSC marker was highly positive in hMSCs, mCBSCs, and sCBSCs but negative in IMPACs phenotypes.

TABLE 7
CBSC Special Diversity in Stem Cell Surface Markers

| Marker | hBMSC | IMPAC 1.0 | IMPAC 2.0 | IMPAC 3.0 | mCBSC | sCBSC |
|---|---|---|---|---|---|---|
| CD11b | 0.56% | 0.64% | 0.14% | 0.14% | 0.06% | 0.59% |
| CD14 | 0.57% | 0.35% | 0.21% | 0.21% | 0.00% | |
| CD19 | 1.61% | 0.30% | 2.17% | 2.02% | 0.02% | |
| CD31 | 4.53% | 0.86% | 1.12% | 1.10% | 0.07% | |
| CD34 | 3.54% | 0.96% | 1.82% | 1.81% | 0.25% | 0.26% |
| CD45 | 1.01% | 0.74% | 3.02% | 2.95% | 0.20% | |
| CD29 | 77.40% | 0.45% | 0.11% | 0.11% | 61.40% | 99.20% |
| CD44 | 98.90% | 1.98% | 2.83% | 1.96% | 93.80% | 97.60% |
| CD73 | 94.50% | 0.36% | 0.88% | 0.84% | 99.50% | |
| CD90/CD90.2 | 50.20% | 0.81% | 1.61% | 2.02% | 0.09% | |
| CD105 | 62.60% | 0.84% | 1.06% | 1.24% | 96.70% | 0.75% |
| CD106 | 17.10% | 0.52% | 1.44% | 1.73% | 91.80% | 6.53% |
| SSEA-1 | 0.34% | 2.51% | 25.50% | 72.80% | 55.80% | |
| SSEA-4 | 36.70% | 0.83% | 27.40% | 2.69% | 22.00% | |
| CD49f | 43.60% | 100.00% | 99.90% | 99.70% | 85.30% | |

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of treating a cardiovascular disease or disorder comprising:
obtaining a biological sample comprising immune modulatory paracrine acting cells (IMPACS);
administering the IMPACS to a subject, wherein the IMPACS produce cardiac cells;
thereby treating the cardiovascular disease or disorder.

2. The method of claim 1, wherein the biological sample comprises bone or fragments thereof.

3. The method of claim 2, wherein the IMPACS are isolated from the bone or fragments thereof.

4. The method of claim 1, wherein the isolated IMPACS are negative for one or more markers comprising: hematopoietic and lineage stem cell markers, CD34, CD117, CD11b, CD31, CD45, major histocompatibility complex (MHC) class II molecules, MHC class Ta molecules, HLA-A, B, C or co-stimulatory molecules.

5. The method of claim 3, wherein the isolated IMPACS express CD49f (integrin alpha chain 6).

6. The method of claim 3, wherein the isolated IMPACS express HLA-DQ.

7. The method of claim 1, wherein the IMPACS express embryonic stem cell surface markers during differentiation.

8. The method of claim 7, wherein the IMPACS express one or more markers during differentiation, wherein the one or more markers comprise SSEA-1, SSEA-4, TRA-1-60 or TRA-1-81.

9. The method of claim 1, wherein the IMPACS modulate immune inflammatory response and immune cell phenotypic shift.

10. The method of claim 1, wherein the IMPACS secrete paracrine factors, exosomes or the combination thereof.

11. The method of claim 10, wherein the IMPACS, secreted paracrine factors, exosomes or combinations thereof, reduce inflammation, rescue stressed muscle cells, and reduce injury induced scar formation.

12. The method of claim 1, further comprising administering to the subject IMPAC exosomal micro RNAs (miRNAs).

13. The method of claim 1, wherein the IMPACS and/or the IMPAC exosomal miRNAs are administrated systemically, locally or the combination thereof.

14. The method of claim 1, wherein the IMPACS and/or the IMPAC exosomal miRNAs are administrated directly to damaged cardiovascular tissues.

15. The method of claim 1, wherein the IMPACS are obtained from sources comprising: autologous, allogeneic, haplotype matched, haplotype mismatched, haplo-identical, xenogeneic or combinations thereof.

16. A composition comprising isolated immune modulatory paracrine acting cells (IMPACS), wherein the isolated IMPACS express CD49f (integrin alpha chain 6), are negative for one or more markers comprising: hematopoietic and lineage stem cell markers, CD34, CD117, CD11b, CD31, CD45, major histocompatibility complex (MHC) class II molecules, MHC class Ia molecules, HLA-A, B, C or co-stimulatory molecules, and the composition comprises one or more factors to maintain the IMPACS in an undifferentiated state.

17. (canceled)

18. The composition of claim 16 wherein the isolated IMPACS express CD49f (integrin alpha chain 6), HLA-DQ.

19. The composition of claim 16, wherein the isolated IMPACS express HLA-DQ.

20. The composition of claim 16, wherein the IMPACS express embryonic stem cell surface markers during differentiation.

21. The composition of claim 20, wherein the IMPACS express one or more markers during differentiation, wherein the one or more markers comprise SSEA-1, SSEA-4, TRA-1-60 or TRA-1-81.

22. The composition of claim 20, wherein the IMPACS express CD49f⁺, SSEA1⁺, SSEA4⁺, CD73⁻, CD90⁻, CD105⁻, CD29⁻, CD44⁻

23. The composition of claim 16, further comprising IMPAC exosomal micro RNAs (miRNAs).

24. The composition of claim 16, wherein the IMPACS are smaller in size when compared to human bone marrow stromal cells (hBMSC).

25. The composition of claim 16, wherein the IMPACS express soluble isoforms of HLA-G.

26. The composition of claim 16, wherein the IMPACS produce one or more immunoregulatory cytokines comprising: IL-4, IL-1RA, TGFβ, IL-10, IL-12a, IL-27β or combinations thereof.

27. A composition comprising isolated immune modulatory paracrine acting cells (IMPACS) and one or more cardiac cell types, comprising: cardiomyocytes (CMs), fibroblasts (FBs), endothelial cells (ECs), peri-vascular cells or combinations thereof.

28. The composition of claim 27, wherein the one or more cardiac cell types.

29. A composition comprising isolated immune modulatory paracrine acting cells (IMPACS) and one or more IMPAC exosomal micro RNAs (miRNAs), wherein the IMPAC exosomal miRNAs specifically inhibit one or more factors comprising: pro-inflammatory cytokines, anti-inflammatory cytokines, cytokines, transcription factors, toll-like receptor (TLR) pathway signaling molecules, NF-κB nuclear transporter, co-stimulatory molecules or combinations thereof.

30. The composition of claim.

31. The composition of claim 29, wherein the pro-inflammatory cytokines comprise IL-6, TNF, IL-1α, IL-1β or combinations thereof, and the anti-inflammatory cytokines comprise TGFβ2, TGFβ3 or the combination thereof.

32. (canceled)

33. The composition of claim 29, wherein the cytokines comprise IL-2, IL-7, IL-13, IL-15 or the combination thereof, wherein the transcription factors comprise: STAT1, STAT2, STAT3, STAT4, STAT5, STAT6 or combinations thereof, wherein the TLR pathway signaling molecules comprise IRAK1, IRAK3, TRAF6, PDCD4, NF-kB1 or combinations thereof, wherein the transcription factors comprise: STAT1, STAT2, STAT3, STAT4, STAT5, STAT6 or combinations thereof, wherein the TLR pathway signaling molecules comprise IRAK1, IRAK3, TRAF6, PDCD4, NF-kB1 or combinations thereof, wherein the NF-κB nuclear transporter is Importin-3a, and, wherein the pro-inflammatory and co-stimulatory molecule is CD40LG.

34. (canceled)

35. (canceled)

36. (canceled)

37. (canceled)

38. (canceled)

39. (canceled)

40. A immune modulatory paracrine acting cell (IMPAC) conditioned medium comprising one or more IMPAC exosomal micro RNAs (miRNAs), paracrine factors.

41. (canceled)

42. A method of promoting wound healing in a subject in need thereof, modulating an immune inflammatory response in wounded cardiac tissues and regeneration of these wounded tissues, comprising: administering a composition comprising immune modulatory paracrine acting cells (IMPACS), IMPAC conditioned compositions or the combination thereof to a subject, wherein the composition promotes the production of new tissue, thereby promoting wound healing.

43. The method of claim 40, wherein the IMPAC conditioned medium comprises one or more IMPAC exosomal micro RNAs (miRNAs), paracrine factors.

44. The method of claim 40, the compositions modulate the immune inflammatory response.

45. The method of claim 40, the compositions reduce death of myocytes and induce cell cycle activity.
